SEQUENCE LISTING (557 sequences)

SEQ ID NO: 1 - DNA, 5756 bp, synthetic construct
  source 1..5756: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaaccgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgatccgaggagatctgccgccgcgatcgccggcgcgccatggcggcggcggccaagcccaacaacctttccctggtggtgcacggaccgggggacttgcgcctggagaactatcctatccctgaaccaggcccaaatgaggtcttgctgaggatgcattctgttggaatctgtggctcagatgtccactactgggagtatggtcgaattgggaattttattgtgaaaaagcccatggtgctgggacatgaagcttcgggaacagtcgaaaaagtgggatcatcggtaaagcacctaaaaccaggtgatcgtgttgccatcgagcctggtgctccccgagaaaatgatgaattctgcaagatgggccgatacaatctgtcaccttccatcttcttctgtgccacgccccccgatgacgggaacctctgccggttctataagcacaatgcagccttttgttacaagcttcctgacaatgtcacctttgaggaaggcgccctgatcgagccactttctgtggggatccatgcctgcaggagaggcggagttaccctgggacacaaggtccttgtgtgtggagctgggccaatcgggatggtcactttgctcgtggccaaagcaatgggagcagctcaagtagtggtgactgatctgtctgctacccgattgtccaaagccaaggagattggggctgatttagtcctccagatctccaaggagagccctcaggaaatcgccaggaaagtagaaggtcagctggggtgcaagccggaagtcaccatcgagtgcacgggggcagaggcctccatccaggcgggcatctacgccactcgctctggtgggaacctcgtgcttgtggggctgggctctgagatgaccaccgtacccctactgcatgcagccatccgggaggtggatatcaagggcgtgtttcgatactgcaacacgtggccagtggcgatttcgatgcttgcgtccaagtctgtgaatgtaaaacccctcgtcacccataggtttcctctggagaaagctctggaggcctttgaaacatttaaaaagggattggggttgaaaatcatgctcaagtgtgaccccagtgaccagaatccctgaagatctcaagcttaactagctagcggaccgacgcgtacgcggccgctcgtttaaacggccggccggatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 18 bp, synthetic construct
  source 1..18: mol_type = other DNA
caggctggcacaaaggag

SEQ ID NO: 3 - DNA, 20 bp, synthetic construct
  source 1..20: mol_type = other DNA
agtgaggcaggatcggtatg

SEQ ID NO: 4 - DNA, 20 bp, synthetic construct
  source 1..20: mol_type = other DNA
agcgtgccatttagcgtatc

SEQ ID NO: 5 - DNA, 26 bp, synthetic construct
  source 1..26: mol_type = other DNA
gcagtagactctgttctcagcctaac

SEQ ID NO: 6 - DNA, 28 bp, synthetic construct
  source 1..28: mol_type = other DNA
accttttctcataaatagatacgaatcc

SEQ ID NO: 7 - DNA, 20 bp, synthetic construct
  source 1..20: mol_type = other DNA
tcttgttccctgctgtaccc

SEQ ID NO: 8 - DNA, 20 bp, synthetic construct
  source 1..20: mol_type = other DNA
gcatgcaagccttcataaca

SEQ ID NO: 9 - DNA, 22 bp, synthetic construct
  source 1..22: mol_type = other DNA
cgaggtcattgttgttatgacg

SEQ ID NO: 10 - DNA, 20 bp, synthetic construct
  source 1..20: mol_type = other DNA
cgtggccatgttaactcctt

SEQ ID NO: 11 - DNA, 20 bp, synthetic construct
  source 1..20: mol_type = other DNA
gttccctgaattcccagtca

SEQ ID NO: 12 - DNA, 27 bp, synthetic construct
  source 1..27: mol_type = other DNA
atgtttaatatttcacgaacatattcc

SEQ ID NO: 13 - DNA, 20 bp, synthetic construct
  source 1..20: mol_type = other DNA
gctgtttcccagtcaaggag

SEQ ID NO: 14 - DNA, 25 bp, synthetic construct
  source 1..25: mol_type = other DNA
tgagtcatcagatttctcttgtttg

SEQ ID NO: 15 - DNA, 20 bp, synthetic construct
  source 1..20: mol_type = other DNA
agcctgggcgactgagtgag

SEQ ID NO: 16 - DNA, 25 bp, synthetic construct
  source 1..25: mol_type = other DNA
aaaagaaaacatagatggcaaaaga

SEQ ID NO: 17 - DNA, 20 bp, synthetic construct
  source 1..20: mol_type = other DNA
tcccgctcagttaagtttgg

SEQ ID NO: 18 - DNA, 20 bp, synthetic construct
  source 1..20: mol_type = other DNA
gcttcaaaatcccctccttc

SEQ ID NO: 19 - DNA, 20 bp, synthetic construct
  source 1..20: mol_type = other DNA
cacctggctctttcctcttg

SEQ ID NO: 20 - DNA, 19 bp, synthetic construct
  source 1..19: mol_type = other DNA
ccctgagatcccaagactg

SEQ ID NO: 21 - RNA, 20 nt, Homo sapiens
  source 1..20: mol_type = unassigned RNA
aggtggagatgatcttaaac

SEQ ID NO: 22 - DNA, 20 bp, synthetic construct
  source 1..20: mol_type = other DNA
  modified_base 1..5: mod_base = OTHER; note = 2'-O-methoxyethyl nucleotide
  modified_base 7: mod_base = OTHER; note = 2'-O-methyl nucleotide
  modified_base 16..20: mod_base = OTHER; note = 2'-O-methoxyethyl nucleotide
  modified_base 1..20: mod_base = OTHER; note = Phosphorothioate linkage
gtttaagatcatctccacct

SEQ ID NO: 23 - RNA, 20 nt, Homo sapiens
  source 1..20: mol_type = unassigned RNA
ggtggagatgatcttaaaca

SEQ ID NO: 24 - DNA, 20 bp, synthetic construct
  source 1..20: mol_type = other DNA
tgtttaagatcatctccacc

SEQ ID NO: 25 - RNA, 20 nt, Homo sapiens
  source 1..20: mol_type = unassigned RNA
tgatcttaaacaaacctggc

SEQ ID NO: 26 - DNA, 20 bp, synthetic construct
  source 1..20: mol_type = other DNA
gccaggtttgtttaagatca

SEQ ID NO: 27 - RNA, 20 nt, Homo sapiens
  source 1..20: mol_type = unassigned RNA
taaacaaacctggcttgaag

SEQ ID NO: 28 - DNA, 20 bp, synthetic construct
  source 1..20: mol_type = other DNA
cttcaagccaggtttgttta

SEQ ID NO: 29 - RNA, 22 nt, Homo sapiens
  source 1..22: mol_type = unassigned RNA
caggtggagatgatcttaaaca

SEQ ID NO: 30 - DNA, 22 bp, synthetic construct
  source 1..22: mol_type = other DNA
tgtttaagatcatctccacctg

SEQ ID NO: 31 - RNA, 22 nt, Homo sapiens
  source 1..22: mol_type = unassigned RNA
gatcttaaacaaacctggcttg

SEQ ID NO: 32 - DNA, 22 bp, synthetic construct
  source 1..22: mol_type = other DNA
caagccaggtttgtttaagatc

SEQ ID NO: 33 - RNA, 22 nt, Homo sapiens
  source 1..22: mol_type = unassigned RNA
cttaaacaaacctggcttgaag

SEQ ID NO: 34 - DNA, 22 bp, synthetic construct
  source 1..22: mol_type = other DNA
cttcaagccaggtttgtttaag

SEQ ID NO: 35 - RNA, 22 nt, Homo sapiens
  source 1..22: mol_type = unassigned RNA
aagtataagcctgcagttaacc

SEQ ID NO: 36 - DNA, 22 bp, synthetic construct
  source 1..22: mol_type = other DNA
ggttaactgcaggcttatactt

SEQ ID NO: 37 - RNA, 22 nt, Homo sapiens
  source 1..22: mol_type = unassigned RNA
tcaaggcgatcgcagccaagca

SEQ ID NO: 38 - DNA, 22 bp, synthetic construct
  source 1..22: mol_type = other DNA
tgcttggctgcgatcgccttga

SEQ ID NO: 39 - RNA, 22 nt, Homo sapiens
  source 1..22: mol_type = unassigned RNA
aaggcgatcgcagccaagcaca

SEQ ID NO: 40 - DNA, 22 bp, synthetic construct
  source 1..22: mol_type = other DNA
tgtgcttggctgcgatcgcctt

SEQ ID NO: 41 - RNA, 22 nt, Homo sapiens
  source 1..22: mol_type = unassigned RNA
acctgtgtttcttgcctcattt

SEQ ID NO: 42 - DNA, 22 bp, synthetic construct
  source 1..22: mol_type = other DNA
aaatgaggcaagaaacacaggt

SEQ ID NO: 43 - DNA, 73 bp, synthetic construct
  source 1..73: mol_type = other DNA
gtagatgcccgcctggatggaggcctctgcccccgtgcactcgatggtgacttccggcttgcaccccagctga

SEQ ID NO: 44 - DNA, 72 bp, synthetic construct
  source 1..72: mol_type = other DNA
gtagatgcctgcctggatggaggcctctgccccgtgcactcgatggtgacttccggcttgcaccccagcaga

SEQ ID NO: 45 - DNA, 1074 bp, Homo sapiens
  source 1..1074: mol_type = genomic DNA
atggcggcggcggccaagcccaacaacctttccctggtggtgcacggaccgggggacttgcgcctggagaactatcctatccctgaaccaggcccaaatgaggtcttgctgaggatgcattctgttggaatctgtggctcagatgtccactactgggagtatggtcgaattgggaattttattgtgaaaaagcccatggtgctgggacatgaagcttcgggaacagtcgaaaaagtgggatcatcggtaaagcacctaaaaccaggtgatcgtgttgccatcgagcctggtgctccccgagaaaatgatgaattctgcaagatgggccgatacaatctgtcaccttccatcttcttctgtgccacgccccccgatgacgggaacctctgccggttctataagcacaatgcagccttttgttacaagcttcctgacaatgtcacctttgaggaaggcgccctgatcgagccactttctgtggggatccatgcctgcaggagaggcggagttaccctgggacacaaggtccttgtgtgtggagctgggccaatcgggatggtcactttgctcgtggccaaagcaatgggagcagctcaagtagtggtgactgatctgtctgctacccgattgtccaaagccaaggagattggggctgatttagtcctccagatctccaaggagagccctcaggaaatcgccaggaaagtagaaggtcagctggggtgcaagccggaagtcaccatcgagtgcacgggggcagaggcctccatccaggcgggcatctacgccactcgctctggtgggaacctcgtgcttgtggggctgggctctgagatgaccaccgtacccctactgcatgcagccatccgggaggtggatatcaagggcgtgtttcgatactgcaacacgtggccagtggcgatttcgatgcttgcgtccaagtctgtgaatgtaaaacccctcgtcacccataggtttcctctggagaaagctctggaggcctttgaaacatttaaaaagggattggggttgaaaatcatgctcaagtgtgaccccagtgaccagaatccctga

SEQ ID NO: 46 - protein, 357 aa, Homo sapiens
MAAAAKPNNLSLVVHGPGDLRLENYPIPEPGPNEVLLRMHSVGICGSDVHYWEYGRIGNFIVKKPMVLGHEASGTVEKVGSSVKHLKPGDRVAIEPGAPRENDEFCKMGRYNLSPSIFFCATPPDDGNLCRFYKHNAAFCYKLPDNVTFEEGALIEPLSVGIHACRRGGVTLGHKVLVCGAGPIGMVTLLVAKAMGAAQVVVTDLSATRLSKAKEIGADLVLQISKESPQEIARKVEGQLGCKPEVTIECTGAEASIQAGIYATRSGGNLVLVGLGSEMTTVPLLHAAIREVDIKGVFRYCNTWPVAISMLASKSVNVKPLVTHRFPLEKALEAFETFKKGLGLKIMLKCDPSDQNP

SEQ ID NO: 47 - DNA, 20 bp, synthetic construct
  source 1..20: mol_type = other DNA
  modified_base 1..5: mod_base = OTHER; note = 2'-O-methoxyethyl nucleotide
  modified_base 7: mod_base = OTHER; note = 2'-O-methyl nucleotide
  modified_base 16..20: mod_base = OTHER; note = 2'-O-methoxyethyl nucleotide
  modified_base 1..20: mod_base = OTHER; note = Phosphorothioate linkage
tattacagctacatcctgtc

SEQ ID NO: 48 - DNA, 5756 bp, Homo sapiens; identical to SEQ ID NO: 1
  source 1..5756: mol_type = genomic DNA

SEQ ID NO: 49 - RNA, 20 nt, Homo sapiens
  source 1..20: mol_type = unassigned RNA
tcgcagccaagcacaataaa

SEQ ID NO: 50 - DNA, 20 bp, synthetic construct
  source 1..20: mol_type = other DNA
tttattgtgcttggctgcga

SEQ ID NO: 51 - RNA, 20 nt, Homo sapiens
  source 1..20: mol_type = unassigned RNA
gcagccaagcacaataaaac

SEQ ID NO: 52 - DNA, 20 bp, synthetic construct
  source 1..20: mol_type = other DNA
gttttattgtgcttggctgc

SEQ ID NO: 53 - RNA, 20 nt, Homo sapiens
  source 1..20: mol_type = unassigned RNA
gatcgcagccaagcacaata

SEQ ID NO: 54 - DNA, 20 bp, synthetic construct
  source 1..20: mol_type = other DNA
tattgtgcttggctgcgatc

SEQ ID NO: 55 - RNA, 20 nt, Homo sapiens
  source 1..20: mol_type = unassigned RNA
atcgcagccaagcacaataa

SEQ ID NO: 56 - DNA, 20 bp, synthetic construct
  source 1..20: mol_type = other DNA
ttattgtgcttggctgcgat

SEQ ID NO: 57 - RNA, 20 nt, Homo sapiens
  source 1..20: mol_type = unassigned RNA
cgcagccaagcacaataaaa

SEQ ID NO: 58 - DNA, 20 bp, synthetic construct
  source 1..20: mol_type = other DNA
ttttattgtgcttggctgcg

SEQ ID NO: 59 - RNA, 20 nt, Homo sapiens
  source 1..20: mol_type = unassigned RNA
gccaagcacaataaaactac

SEQ ID NO: 60 - DNA, 20 bp, synthetic construct
  source 1..20: mol_type = other DNA
gtagttttattgtgcttggc

SEQ ID NO: 61 - RNA, 20 nt, Homo sapiens
  source 1..20: mol_type = unassigned RNA
cgatcgcagccaagcacaat

SEQ ID NO: 62 - DNA, 20 bp, synthetic construct
  source 1..20: mol_type = other DNA
attgtgcttggctgcgatcg

SEQ ID NO: 63 - RNA, 20 nt, Homo sapiens
  source 1..20: mol_type = unassigned RNA
agcacaataaaactacagcc

SEQ ID NO: 64 - DNA, 20 bp, synthetic construct
  source 1..20: mol_type = other DNA
ggctgtagttttattgtgct

SEQ ID NO: 65 - RNA, 20 nt, Homo sapiens
  source 1..20: mol_type = unassigned RNA
cagccaagcacaataaaact

SEQ ID NO: 66 - DNA, 20 bp, synthetic construct
  source 1..20: mol_type = other DNA
agttttattgtgcttggctg

SEQ ID NO: 67 - RNA, 20 nt, Homo sapiens
  source 1..20: mol_type = unassigned RNA
gcgatcgcagccaagcacaa

SEQ ID NO: 68 - DNA, 20 bp, synthetic construct
  source 1..20: mol_type = other DNA
ttgtgcttggctgcgatcgc

SEQ ID NO: 69 - RNA, 20 nt, Homo sapiens
  source 1..20: mol_type = unassigned RNA
gcacaataaaactacagccc

SEQ ID NO: 70 - DNA, 20 bp, synthetic construct
  source 1..20: mol_type = other DNA
gggctgtagttttattgtgc

SEQ ID NO: 71 - RNA, 20 nt, Homo sapiens
  source 1..20: mol_type = unassigned RNA
aaccagattgagtgccaccc

SEQ ID NO: 72 - DNA, 20 bp, synthetic construct
  source 1..20: mol_type = other DNA
gggtggcactcaatctggtt

SEQ ID NO: 73 - RNA, 20 nt, Homo sapiens
  source 1..20: mol_type = unassigned RNA
accagattgagtgccaccca

SEQ ID NO: 74 - DNA, 20 bp, synthetic construct
  source 1..20: mol_type = other DNA
tgggtggcactcaatctggt

SEQ ID NO: 75 - RNA, 20 nt, Homo sapiens
  source 1..20: mol_type = unassigned RNA
acctcccacaaggattaccc

SEQ ID NO: 76 - DNA, 20 bp, synthetic construct
  source 1..20: mol_type = other DNA
gggtaatccttgtgggaggt

SEQ ID NO: 77 - RNA, 20 nt, Homo sapiens
  source 1..20: mol_type = unassigned RNA
cctcccacaaggattacccc

SEQ ID NO: 78 - DNA, 20 bp, synthetic construct
  source 1..20: mol_type = other DNA
ggggtaatccttgtgggagg

SEQ ID NO: 79 - RNA, 20 nt, Homo sapiens
  source 1..20: mol_type = unassigned RNA
acaacctgaatacccttttc

SEQ ID NO: 80 - DNA, 20 bp, synthetic construct
  source 1..20: mol_type = other DNA
gaaaagggtattcaggttgt

SEQ ID NO: 81 - RNA, 20 nt, Homo sapiens
  source 1..20: mol_type = unassigned RNA
ctcccacaaggattacccct

SEQ ID NO: 82 - DNA, 20 bp, synthetic construct
  source 1..20: mol_type = other DNA
aggggtaatccttgtgggag

SEQ ID NO: 83 - RNA, 20 nt, Homo sapiens
  source 1..20: mol_type = unassigned RNA
ccagattgagtgccacccat

SEQ ID NO: 84 - DNA, 20 bp, synthetic construct
  source 1..20: mol_type = other DNA
atgggtggcactcaatctgg

SEQ ID NO: 85 - RNA, 20 nt, Homo sapiens
  source 1..20: mol_type = unassigned RNA
caacctgaatacccttttct

SEQ ID NO: 86 - DNA, 20 bp, synthetic construct
  source 1..20: mol_type = other DNA
agaaaagggtattcaggttg

SEQ ID NO: 87 - RNA, 20 nt, Homo sapiens
  source 1..20: mol_type = unassigned RNA
agtacaacctgaataccctt

SEQ ID NO: 88 - DNA, 20 bp, synthetic construct
  source 1..20: mol_type = other DNA
aagggtattcaggttgtact

SEQ ID NO: 89 - RNA, 20 nt, Homo sapiens
  source 1..20: mol_type = unassigned RNA
gtacaacctgaatacccttt

SEQ ID NO: 90 - DNA, 20 bp, synthetic construct
  source 1..20: mol_type = other DNA
aaagggtattcaggttgtac

SEQ ID NO: 91 - RNA, 20 nt, Homo sapiens
  source 1..20: mol_type = unassigned RNA
aaggcgatcgcagccaagca

SEQ ID NO: 92 - DNA, 20 bp, synthetic construct
  source 1..20: mol_type = other DNA
tgcttggctgcgatcgcctt

SEQ ID NO: 93 - RNA, 20 nt, Homo sapiens
  source 1..20: mol_type = unassigned RNA
tcccacaaggattacccctt

SEQ ID NO: 94 - DNA, 20 bp, synthetic construct
  source 1..20: mol_type = other DNA
aaggggtaatccttgtggga

SEQ ID NO: 95 - RNA, 20 nt, Homo sapiens
  source 1..20: mol_type = unassigned RNA
aagtacaacctgaataccct

SEQ ID NO: 96 - DNA, 20 bp, synthetic construct
  source 1..20: mol_type = other DNA
agggtattcaggttgtactt

SEQ ID NO: 97 - RNA, 20 nt, Homo sapiens
  source 1..20: mol_type = unassigned RNA
aacctgaatacccttttctg

SEQ ID NO: 98 - DNA, 20 bp, synthetic construct
  source 1..20: mol_type = other DNA
cagaaaagggtattcaggtt

SEQ ID NO: 99 - RNA, 20 nt, Homo sapiens
  source 1..20: mol_type = unassigned RNA
acctgaatacccttttctga

SEQ ID NO: 100 - DNA, 20 bp, synthetic construct
  source 1..20: mol_type = other DNA
tcagaaaagggtattcaggt

SEQ ID NO: 101 - RNA, 20 nt, Homo sapiens
  source 1..20: mol_type = unassigned RNA
tacctcccacaaggattacc

SEQ ID NO: 102 - DNA, 20 bp, synthetic construct
  source 1..20: mol_type = other DNA
ggtaatccttgtgggaggta

SEQ ID NO: 103 - RNA, 20 nt, Homo sapiens
  source 1..20: mol_type = unassigned RNA
aaagtacaacctgaataccc

SEQ ID NO: 104 - DNA, 20 bp, synthetic construct
  source 1..20: mol_type = other DNA
gggtattcaggttgtacttt

SEQ ID NO: 105 - RNA, 20 nt, Homo sapiens
  source 1..20: mol_type = unassigned RNA
cctgaatacccttttctgac

SEQ ID NO: 106 - DNA, 20 bp, synthetic construct
  source 1..20: mol_type = other DNA
gtcagaaaagggtattcagg

SEQ ID NO: 107 - RNA, 20 nt, Homo sapiens
  source 1..20: mol_type = unassigned RNA
taaccagattgagtgccacc

SEQ ID NO: 108 - DNA, 20 bp, synthetic construct
  source 1..20: mol_type = other DNA
ggtggcactcaatctggtta

SEQ ID NO: 109 - RNA, 20 nt, Homo sapiens
  source 1..20: mol_type = unassigned RNA
attgagtgccacccatatct

SEQ ID NO: 110 - DNA, 20 bp, synthetic construct
  source 1..20: mol_type = other DNA
agatatgggtggcactcaat

SEQ ID NO: 111 - RNA, 20 nt, Homo sapiens
  source 1..20: mol_type = unassigned RNA
gtgccacccatatctcactc

SEQ ID NO: 112 - DNA, 20 bp, synthetic construct
  source 1..20: mol_type = other DNA
gagtgagatatgggtggcac

SEQ ID NO: 113 - RNA, 20 nt, Homo sapiens
  source 1..20: mol_type = unassigned RNA
tgccacccatatctcactca

SEQ ID NO: 114 - DNA, 20 bp, synthetic construct
  source 1..20: mol_type = other DNA
tgagtgagatatgggtggca

SEQ ID NO: 115 - RNA, 20 nt, Homo sapiens
  source 1..20: mol_type = unassigned RNA
cacaataaaactacagccca

SEQ ID NO: 116 - DNA, 20 bp, synthetic construct
  source 1..20: mol_type = other DNA
tgggctgtagttttattgtg

SEQ ID NO: 117 - RNA, 20 nt, Homo sapiens
  source 1..20: mol_type = unassigned RNA
cccacaaggattaccccttc

SEQ ID NO: 118 - DNA, 20 bp, synthetic construct
  source 1..20: mol_type = other DNA
gaaggggtaatccttgtggg

SEQ ID NO: 119 - RNA, 20 nt, Homo sapiens
  source 1..20: mol_type = unassigned RNA
ctgaatacccttttctgacc

SEQ ID NO: 120 - DNA, 20 bp, synthetic construct
  source 1..20: mol_type = other DNA
ggtcagaaaagggtattcag

SEQ ID NO: 121 - RNA, 20 nt, Homo sapiens
  source 1..20: mol_type = unassigned RNA
gattgagtgccacccatatc

SEQ ID NO: 122 - DNA, 20 bp, synthetic construct
  source 1..20: mol_type = other DNA
gatatgggtggcactcaatc

SEQ ID NO: 123 - RNA, 20 nt, Homo sapiens
  source 1..20: mol_type = unassigned RNA
agtgccacccatatctcact

SEQ ID NO: 124 - DNA, 20 bp, synthetic construct
  source 1..20: mol_type = other DNA
agtgagatatgggtggcact

SEQ ID NO: 125 - RNA, 20 nt, Homo sapiens
  source 1..20: mol_type = unassigned RNA
gccacccatatctcactcag

SEQ ID NO: 126 - DNA, 20 bp, synthetic construct
  source 1..20: mol_type = other DNA
ctgagtgagatatgggtggc

SEQ ID NO: 127 - RNA, 20 nt, Homo sapiens
  source 1..20: mol_type = unassigned RNA
gagtgccacccatatctcac

SEQ ID NO: 128 - DNA, 20 bp, synthetic construct
  source 1..20: mol_type = other DNA
gtgagatatgggtggcactc

SEQ ID NO: 129 - RNA, 20 nt, Homo sapiens
  source 1..20: mol_type = unassigned RNA
tgaatacccttttctgacca

SEQ ID NO: 130 - DNA, 20 bp, synthetic construct
  source 1..20: mol_type = other DNA
tggtcagaaaagggtattca

SEQ ID NO: 131 - RNA, 20 nt, Homo sapiens
  source 1..20: mol_type = unassigned RNA
ttgagtgccacccatatctc

SEQ ID NO: 132 - DNA, 20 bp, synthetic construct
  source 1..20: mol_type = other DNA
gagatatgggtggcactcaa

SEQ ID NO: 133 - RNA, 20 nt, Homo sapiens
  source 1..20: mol_type = unassigned RNA
tgagtgccacccatatctca

SEQ ID NO: 134 - DNA, 20 bp, synthetic construct
  source 1..20: mol_type = other DNA
tgagatatgggtggcactca

SEQ ID NO: 135 - RNA, 20 nt, Homo sapiens
  source 1..20: mol_type = unassigned RNA
agattgagtgccacccatat

SEQ ID NO: 136 - DNA, 20 bp, synthetic construct
  source 1..20: mol_type = other DNA
atatgggtggcactcaatct

SEQ ID NO: 137 - RNA, 20 nt, Homo sapiens
  source 1..20: mol_type = unassigned RNA
ccacccatatctcactcagg

SEQ ID NO: 138 - DNA, 20 bp, synthetic construct
  source 1..20: mol_type = other DNA
cctgagtgagatatgggtgg

SEQ ID NO: 139 - RNA, 20 nt, Homo sapiens
  source 1..20: mol_type = unassigned RNA
cagattgagtgccacccata

SEQ ID NO: 140 - DNA, 20 bp, synthetic construct
  source 1..20: mol_type = other DNA
tatgggtggcactcaatctg

SEQ ID NO: 141 - RNA, 20 nt, Homo sapiens
  source 1..20: mol_type = unassigned RNA
ccacaaggattaccccttcc

SEQ ID NO: 142 - DNA, 20 bp, synthetic construct
  source 1..20: mol_type = other DNA
ggaaggggtaatccttgtgg

SEQ ID NO: 143 - RNA, 20 nt, Homo sapiens
  source 1..20: mol_type = unassigned RNA
gaaagtacaacctgaatacc

SEQ ID NO: 144 - DNA, 20 bp, synthetic construct
  source 1..20: mol_type = other DNA
ggtattcaggttgtactttc

SEQ ID NO: 145 - RNA, 20 nt, Homo sapiens
  source 1..20: mol_type = unassigned RNA
gaatacccttttctgaccaa

SEQ ID NO: 146 - DNA, 20 bp, synthetic construct
  source 1..20: mol_type = other DNA
ttggtcagaaaagggtattc

SEQ ID NO: 147 - RNA, 20 nt, Homo sapiens
  source 1..20: mol_type = unassigned RNA
acaataaaactacagcccag

SEQ ID NO: 148 - DNA, 20 bp, synthetic construct
  source 1..20: mol_type = other DNA
ctgggctgtagttttattgt

SEQ ID NO: 149 - RNA, 20 nt, Homo sapiens
  source 1..20: mol_type = unassigned RNA
gtacctcccacaaggattac

SEQ ID NO: 150 - DNA, 20 bp, synthetic construct
  source 1..20: mol_type = other DNA
gtaatccttgtgggaggtac

SEQ ID NO: 151 - RNA, 20 nt, Homo sapiens
  source 1..20: mol_type = unassigned RNA
aaacaaacctggcttgaagt

SEQ ID NO: 152 - DNA, 20 bp, synthetic construct
  source 1..20: mol_type = other DNA
acttcaagccaggtttgttt

SEQ ID NO: 153 - RNA, 20 nt, Homo sapiens
  source 1..20: mol_type = unassigned RNA
tcaaggcgatcgcagccaag

SEQ ID NO: 154 - DNA, 20 bp, synthetic construct
  source 1..20: mol_type = other DNA
cttggctgcgatcgccttga

SEQ ID NO: 155 - RNA, 20 nt, Homo sapiens
  source 1..20: mol_type = unassigned RNA
atggcaagccgtctcctgct

SEQ ID NO: 156 - DNA, 20 bp, synthetic construct
  source 1..20: mol_type = other DNA
agcaggagacggcttgccat

SEQ ID NO: 157 - RNA, 20 nt, Homo sapiens
  source 1..20: mol_type = unassigned RNA
gatcttaaacaaacctggct

SEQ ID NO: 158 - DNA, 20 bp, synthetic construct
  source 1..20: mol_type = other DNA
agccaggtttgtttaagatc

SEQ ID NO: 159 - RNA, 20 nt, Homo sapiens
  source 1..20: mol_type = unassigned RNA
tgatcttaaacaaacctggc

SEQ ID NO: 160 - DNA, 20 bp, synthetic construct
  source 1..20: mol_type = other DNA
gccaggtttgtttaagatca

SEQ ID NO: 161 - RNA, 20 nt, Homo sapiens
  source 1..20: mol_type = unassigned RNA
taaacaaacctggcttgaag

SEQ ID NO: 162 - DNA, 20 bp, synthetic construct
  source 1..20: mol_type = other DNA
cttcaagccaggtttgttta

SEQ ID NO: 163 - RNA, 20 nt, Homo sapiens
  source 1..20: mol_type = unassigned RNA
aacaaacctggcttgaagta

SEQ ID NO: 164 - DNA, 20 bp, synthetic construct
  source 1..20: mol_type = other DNA
tacttcaagccaggtttgtt

SEQ ID NO: 165 - RNA, 20 nt, Homo sapiens
  source 1..20: mol_type = unassigned RNA
ctcagcagtgggacagcaac

SEQ ID NO: 166 - DNA, 20 bp, synthetic construct
  source 1..20: mol_type = other DNA
gttgctgtcccactgctgag

SEQ ID NO: 167 - RNA, 20 nt, Homo sapiens
  source 1..20: mol_type = unassigned RNA
tcagcagtgggacagcaacc

SEQ ID NO: 168 - DNA, 20 bp, synthetic construct
  source 1..20: mol_type = other DNA
ggttgctgtcccactgctga

SEQ ID NO: 169 - RNA, 20 nt, Homo sapiens
  source 1..20: mol_type = unassigned RNA
cagcagtgggacagcaacct

SEQ ID NO: 170 - DNA, 20 bp, synthetic construct
  source 1..20: mol_type = other DNA
aggttgctgtcccactgctg

SEQ ID NO: 171 - RNA, 20 nt, Homo sapiens
  source 1..20: mol_type = unassigned RNA
agcagtgggacagcaacctg

SEQ ID NO: 172 - DNA, 20 bp, synthetic construct
  source 1..20: mol_type = other DNA
caggttgctgtcccactgct

SEQ ID NO: 173 - RNA, 20 nt, Homo sapiens
  source 1..20: mol_type = unassigned RNA
gcagtgggacagcaacctgt

SEQ ID NO: 174 - DNA, 20 bp, synthetic construct
  source 1..20: mol_type = other DNA
acaggttgctgtcccactgc

SEQ ID NO: 175 - RNA, 20 nt, Homo sapiens
  source 1..20: mol_type = unassigned RNA
cagtgggacagcaacctgta

SEQ ID NO: 176 - DNA, 20 bp, synthetic construct
  source 1..20: mol_type = other DNA
tacaggttgctgtcccactg

SEQ ID NO: 177 - RNA, 20 nt, Homo sapiens
  source 1..20: mol_type = unassigned RNA
agtgggacagcaacctgtag

SEQ ID NO: 178 - DNA, 20 bp, synthetic construct
  source 1..20: mol_type = other DNA
ctacaggttgctgtcccact

SEQ ID NO: 179 - RNA, 20 nt, Homo sapiens
  source 1..20: mol_type = unassigned RNA
tgtacctcccacaaggatta

SEQ ID NO: 180 - DNA, 20 bp, synthetic construct
  source 1..20: mol_type = other DNA
taatccttgtgggaggtaca

SEQ ID NO: 181 - RNA, 20 nt, Homo sapiens
  source 1..20: mol_type = unassigned RNA
aagtgacctatacctgtgtt

SEQ ID NO: 182 - DNA, 20 bp, synthetic construct
  source 1..20: mol_type = other DNA
aacacaggtataggtcactt

SEQ ID NO: 183 - RNA, 20 nt, Homo sapiens
  source 1..20: mol_type = unassigned RNA
gtgggacagcaacctgtaga

SEQ ID NO: 184 - DNA, 20 bp, synthetic construct
  source 1..20: mol_type = other DNA
tctacaggttgctgtcccac

SEQ ID NO: 185 - RNA, 20 nt, Homo sapiens
  source 1..20: mol_type = unassigned RNA
tagtgccactaacggttgag

SEQ ID NO: 186 - DNA, 20 bp, synthetic construct
  source 1..20: mol_type = other DNA
ctcaaccgttagtggcacta

SEQ ID NO: 187 - RNA, 20 nt, Homo sapiens
  source 1..20: mol_type = unassigned RNA
agtgccactaacggttgagt

SEQ ID NO: 188 - DNA, 20 bp, synthetic construct
  source 1..20: mol_type = other DNA
actcaaccgttagtggcact

SEQ ID NO: 189 - RNA, 20 nt, Homo sapiens
  source 1..20: mol_type = unassigned RNA
aggtggagatgatcttaaac

SEQ ID NO: 190 - DNA, 20 bp, synthetic construct
  source 1..20: mol_type = other DNA
gtttaagatcatctccacct

SEQ ID NO: 191 - RNA, 20 nt, Homo sapiens
  source 1..20: mol_type = unassigned RNA
tgggacagcaacctgtagag

SEQ ID NO: 192 - DNA, 20 bp, synthetic construct
  source 1..20: mol_type = other DNA
ctctacaggttgctgtccca

SEQ ID NO: 193 - RNA, 20 nt, Homo sapiens
  source 1..20: mol_type = unassigned RNA
atagtgccactaacggttga

SEQ ID NO: 194 - DNA, 20 bp, synthetic construct
  source 1..20: mol_type = other DNA
tcaaccgttagtggcactat

SEQ ID NO: 195 - RNA, 20 nt, Homo sapiens
  source 1..20: mol_type = unassigned RNA
caagtgacctatacctgtgt

SEQ ID NO: 196 - DNA, 20 bp, synthetic construct
  source 1..20: mol_type = other DNA
acacaggtataggtcacttg

SEQ ID NO: 197 - RNA, 20 nt, Homo sapiens
  source 1..20: mol_type = unassigned RNA
agtgacctatacctgtgttt

SEQ ID NO: 198 - DNA, 20 bp, synthetic construct
  source 1..20: mol_type = other DNA
aaacacaggtataggtcact

SEQ ID NO: 199 - RNA, 20 nt, Homo sapiens
  source 1..20: mol_type = unassigned RNA
gcaagccgtctcctgctcaa

SEQ ID NO: 200 - DNA, 20 bp, synthetic construct
  source 1..20: mol_type = other DNA
ttgagcaggagacggcttgc

SEQ ID NO: 201 - RNA, 20 nt, Homo sapiens
  source 1..20: mol_type = unassigned RNA
caagccgtctcctgctcaac

SEQ ID NO: 202 - DNA, 20 bp, synthetic construct
  source 1..20: mol_type = other DNA
gttgagcaggagacggcttg

SEQ ID NO: 203 - RNA, 20 nt, Homo sapiens
  source 1..20: mol_type = unassigned RNA
aagccgtctcctgctcaaca

SEQ ID NO: 204 - DNA, 20 bp, synthetic construct
  source 1..20: mol_type = other DNA
tgttgagcaggagacggctt

SEQ ID NO: 205 - RNA, 20 nt, Homo sapiens
  source 1..20: mol_type = unassigned RNA
tcttaaacaaacctggcttg

SEQ ID NO: 206 - DNA, 20 bp, synthetic construct
  source 1..20: mol_type = other DNA
caagccaggtttgtttaaga

SEQ ID NO: 207 - RNA, 20 nt, Homo sapiens
  source 1..20: mol_type = unassigned RNA
tctgtgacaccagaacgcat

SEQ ID NO: 208 - DNA, 20 bp, synthetic construct
  source 1..20: mol_type = other DNA
atgcgttctggtgtcacaga

SEQ ID NO: 209 - RNA, 20 nt, Homo sapiens
  source 1..20: mol_type = unassigned RNA
ctgtgacaccagaacgcatt

SEQ ID NO: 210 - DNA, 20 bp, synthetic construct
  source 1..20: mol_type = other DNA
aatgcgttctggtgtcacag

SEQ ID NO: 211 - RNA, 20 nt, Homo sapiens
  source 1..20: mol_type = unassigned RNA
tgtgacaccagaacgcattg

SEQ ID NO: 212 - DNA, 20 bp, synthetic construct
  source 1..20: mol_type = other DNA
caatgcgttctggtgtcaca

SEQ ID NO: 213 - RNA, 20 nt, Homo sapiens
  source 1..20: mol_type = unassigned RNA
aacttcaaccatctccaggt

SEQ ID NO: 214 - DNA, 20 bp, synthetic construct
  source 1..20: mol_type = other DNA
acctggagatggttgaagtt

SEQ ID NO: 215 - RNA, 20 nt, Homo sapiens
  source 1..20: mol_type = unassigned RNA
acaaacctggcttgaagtat

SEQ ID NO: 216 - DNA, 20 bp, synthetic construct
  source 1..20: mol_type = other DNA
atacttcaagccaggtttgt

SEQ ID NO: 217 - RNA, 20 nt, Homo sapiens
  source 1..20: mol_type = unassigned RNA
ttaaccagattgagtgccac

SEQ ID NO: 218 - DNA, 20 bp, synthetic construct
  source 1..20: mol_type = other DNA
gtggcactcaatctggttaa

SEQ ID NO: 219 - RNA, 20 nt, Homo sapiens
  source 1..20: mol_type = unassigned RNA
gtgacaccagaacgcattgc

SEQ ID NO: 220 - DNA, 20 bp, synthetic construct
  source 1..20: mol_type = other DNA
gcaatgcgttctggtgtcac

SEQ ID NO: 221 - RNA, 20 nt, Homo sapiens
  source 1..20: mol_type = unassigned RNA
tgacaccagaacgcattgct

SEQ ID NO: 222 - DNA, 20 bp, synthetic construct
  source 1..20: mol_type = other DNA
agcaatgcgttctggtgtca

SEQ ID NO: 223 - RNA, 20 nt, Homo sapiens
  source 1..20: mol_type = unassigned RNA
attaccccttccatgaagag

SEQ ID NO: 224 - DNA, 20 bp, synthetic construct
  source 1..20: mol_type = other DNA
ctcttcatggaaggggtaat

SEQ ID NO: 225 - RNA, 20 nt, Homo sapiens
  source 1..20: mol_type = unassigned RNA
gggacagcaacctgtagagt

SEQ ID NO: 226 - DNA, 20 bp, synthetic construct
  source 1..20: mol_type = other DNA
actctacaggttgctgtccc

SEQ ID NO: 227 - RNA, 20 nt, Homo sapiens
  source 1..20: mol_type = unassigned RNA
atacccttttctgaccaaag

SEQ ID NO: 228 - DNA, 20 bp, synthetic construct
  source 1..20: mol_type = other DNA
ctttggtcagaaaagggtat

SEQ ID NO: 229 - RNA, 20 nt, Homo sapiens
  source 1..20: mol_type = unassigned RNA
atcaaggcgatcgcagccaa

SEQ ID NO: 230 - DNA, 20 bp, synthetic construct
  source 1..20: mol_type = other DNA
ttggctgcgatcgccttgat

SEQ ID NO: 231 - RNA, 20 nt, Homo sapiens
  source 1..20: mol_type = unassigned RNA
agtctgtgacaccagaacgc

SEQ ID NO: 232 - DNA, 20 bp, synthetic construct
  source 1..20: mol_type = other DNA
gcgttctggtgtcacagact

SEQ ID NO: 233 - RNA, 20 nt, Homo sapiens
  source 1..20: mol_type = unassigned RNA
ctgtacctcccacaaggatt

SEQ ID NO: 234 - DNA, 20 bp, synthetic construct
  source 1..20: mol_type = other DNA
aatccttgtgggaggtacag

SEQ ID NO: 235 - RNA, 20 nt, Homo sapiens
  source 1..20: mol_type = unassigned RNA
cacaaggattaccccttcca

SEQ ID NO: 236 - DNA, 20 bp, synthetic construct
  source 1..20: mol_type = other DNA
tggaaggggtaatccttgtg

SEQ ID NO: 237 - RNA, 20 nt, Homo sapiens
  source 1..20: mol_type = unassigned RNA
gattaccccttccatgaaga

SEQ ID NO: 238 - DNA, 20 bp, synthetic construct
  source 1..20: mol_type = other DNA
tcttcatggaaggggtaatc

SEQ ID NO: 239 - RNA, 20 nt, Homo sapiens
  source 1..20: mol_type = unassigned RNA
ttaccccttccatgaagagt

SEQ ID NO: 240 - DNA, 20 bp, synthetic construct
  source 1..20: mol_type = other DNA
actcttcatggaaggggtaa

SEQ ID NO: 241 - RNA, 20 nt, Homo sapiens
  source 1..20: mol_type = unassigned RNA
taccccttccatgaagagtt

SEQ ID NO: 242 - DNA, 20 bp, synthetic construct
  source 1..20: mol_type = other DNA
aactcttcatggaaggggta

SEQ ID NO: 243 - RNA, 20 nt, Homo sapiens
  source 1..20: mol_type = unassigned RNA
cacccatatctcactcagga

SEQ ID NO: 244 - DNA, 20 bp, synthetic construct
  source 1..20: mol_type = other DNA
tcctgagtgagatatgggtg

SEQ ID NO: 245 - RNA, 20 nt, Homo sapiens
  source 1..20: mol_type = unassigned RNA
ggattaccccttccatgaag

SEQ ID NO: 246 - DNA, 20 bp, synthetic construct
  source 1..20: mol_type = other DNA
cttcatggaaggggtaatcc

SEQ ID NO: 247 - RNA, 20 nt, Homo sapiens
  source 1..20: mol_type = unassigned RNA
actcagcagtgggacagcaa

SEQ ID NO: 248 - DNA, 20 bp, synthetic construct
  source 1..20: mol_type = other DNA
ttgctgtcccactgctgagt

SEQ ID NO: 249 - RNA, 20 nt, Homo sapiens
  source 1..20: mol_type = unassigned RNA
aagcctgcagttaaccagat

SEQ ID NO: 250 - DNA, 20 bp, synthetic construct
  source 1..20: mol_type = other DNA
atctggttaactgcaggctt

SEQ ID NO: 251 - RNA, 20 nt, Homo sapiens
  source 1..20: mol_type = unassigned RNA
gtctgtgacaccagaacgca

SEQ ID NO: 252 - DNA, 20 bp, synthetic construct
  source 1..20: mol_type = other DNA
tgcgttctggtgtcacagac

SEQ ID NO: 253 - RNA, 20 nt, Homo sapiens
  source 1..20: mol_type = unassigned RNA
gctgtacctcccacaaggat

SEQ ID NO: 254 - DNA, 20 bp, synthetic construct
  source 1..20: mol_type = other DNA
atccttgtgggaggtacagc

SEQ ID NO: 255 - RNA, 20 nt, Homo sapiens
  source 1..20: mol_type = unassigned RNA
aggattaccccttccatgaa

SEQ ID NO: 256 - DNA, 20 bp, synthetic construct
  source 1..20: mol_type = other DNA
ttcatggaaggggtaatcct

SEQ ID NO: 257 - RNA, 20 nt, Homo sapiens
  source 1..20: mol_type = unassigned RNA
agcctgcagttaaccagatt

SEQ ID NO: 258 - DNA, 20 bp, synthetic construct
  source 1..20: mol_type = other DNA
aatctggttaactgcaggct

SEQ ID NO: 259 - RNA, 20 nt, Homo sapiens
  source 1..20: mol_type = unassigned RNA
ccaagtgacctatacctgtg

SEQ ID NO: 260 - DNA, 20 bp, synthetic construct
  source 1..20: mol_type = other DNA
cacaggtataggtcacttgg

SEQ ID NO: 261 - RNA, 20 nt, Homo sapiens
  source 1..20: mol_type = unassigned RNA
acttcaaccatctccaggtg

SEQ ID NO: 262 - DNA, 20 bp, synthetic construct
  source 1..20: mol_type = other DNA
cacctggagatggttgaagt

SEQ ID NO: 263 - RNA, 20 nt, Homo sapiens
  source 1..20: mol_type = unassigned RNA
ttgagctgtacctcccacaa

SEQ ID NO: 264 - DNA, 20 bp, synthetic construct
  source 1..20: mol_type = other DNA
ttgtgggaggtacagctcaa

SEQ ID NO: 265 - RNA, 20 nt, Homo sapiens
  source 1..20: mol_type = unassigned RNA
tgagctgtacctcccacaag

SEQ ID NO: 266 - DNA, 20 bp, synthetic construct
  source 1..20: mol_type = other DNA
cttgtgggaggtacagctca

SEQ ID NO: 267 - RNA, 20 nt, Homo sapiens
  source 1..20: mol_type = unassigned RNA
gagctgtacctcccacaagg

SEQ ID NO: 268 - DNA, 20 bp, synthetic construct
  source 1..20: mol_type = other DNA
ccttgtgggaggtacagctc

SEQ ID NO: 269 - RNA, 20 nt, Homo sapiens
  source 1..20: mol_type = unassigned RNA
agctgtacctcccacaagga

SEQ ID NO: 270 - DNA, 20 bp, synthetic construct
  source 1..20: mol_type = other DNA
tccttgtgggaggtacagct

SEQ ID NO: 271 - RNA, 20 nt, Homo sapiens
  source 1..20: mol_type = unassigned RNA
accccttccatgaagagttt

SEQ ID NO: 272 - DNA, 20 bp, synthetic construct
  source 1..20: mol_type = other DNA
aaactcttcatggaaggggt

SEQ ID NO: 273 - RNA, 20 nt, Homo sapiens
  source 1..20: mol_type = unassigned RNA
ggaaagtacaacctgaatac

SEQ ID NO: 274 - DNA, 20 bp, synthetic construct
  source 1..20: mol_type = other DNA
gtattcaggttgtactttcc

SEQ ID NO: 275 - RNA, 20 nt, Homo sapiens
  source 1..20: mol_type = unassigned RNA
ggaatttttcccattggatg

SEQ ID NO: 276 - DNA, 20 bp, synthetic construct
  source 1..20: mol_type = other DNA
catccaatgggaaaaattcc

SEQ ID NO: 277 - RNA, 20 nt, Homo sapiens
  source 1..20: mol_type = unassigned RNA
ggtggagatgatcttaaaca

SEQ ID NO: 278 - DNA, 20 bp, synthetic construct
  source 1..20: mol_type = other DNA
tgtttaagatcatctccacc

SEQ ID NO: 279 - RNA, 20 nt, Homo sapiens
  source 1..20: mol_type = unassigned RNA
gcctgcagttaaccagattg

SEQ ID NO: 280 - DNA, 20 bp, synthetic construct
  source 1..20: mol_type = other DNA
caatctggttaactgcaggc

SEQ ID NO: 281 - RNA, 20 nt, Homo sapiens
  source 1..20: mol_type = unassigned RNA
acaaggattaccccttccat

SEQ ID NO: 282 - DNA, 20 bp, synthetic construct
  source 1..20: mol_type = other DNA
atggaaggggtaatccttgt

SEQ ID NO: 283 - RNA, 20 nt, Homo sapiens
  source 1..20: mol_type = unassigned RNA
agccgtctcctgctcaacaa

SEQ ID NO: 284 - DNA, 20 bp, synthetic construct
  source 1..20: mol_type = other DNA
ttgttgagcaggagacggct

SEQ ID NO: 285 - RNA, 20 nt, Homo sapiens
  source 1..20: mol_type = unassigned RNA
attggatgagtcgggcaatg

SEQ ID NO: 286 - DNA, 20 bp, synthetic construct
  source 1..20: mol_type = other DNA
cattgcccgactcatccaat

SEQ ID NO: 287 - RNA, 20 nt, Homo sapiens
  source 1..20: mol_type = unassigned RNA
taagcctgcagttaaccaga

SEQ ID NO: 288 - DNA, 20 bp, synthetic construct
  source 1..20: mol_type = other DNA
tctggttaactgcaggctta

SEQ ID NO: 289 - RNA, 20 nt, Homo sapiens
  source 1..20: mol_type = unassigned RNA
ccccaagtctgtgacaccag

SEQ ID NO: 290 - DNA, 20 bp, synthetic construct
  source 1..20: mol_type = other DNA
ctggtgtcacagacttgggg

SEQ ID NO: 291 - RNA, 20 nt, Homo sapiens
  source 1..20: mol_type = unassigned RNA
gtgacctatacctgtgtttc

SEQ ID NO: 292 - DNA, 20 bp, synthetic construct
  source 1..20: mol_type = other DNA
gaaacacaggtataggtcac

SEQ ID NO: 293 - RNA, 20 nt, Homo sapiens
  source 1..20: mol_type = unassigned RNA
ggacagcaacctgtagagtg

SEQ ID NO: 294 - DNA, 20 bp, synthetic construct
  source 1..20: mol_type = other DNA
cactctacaggttgctgtcc

SEQ ID NO: 295 - RNA, 20 nt, Homo sapiens
  source 1..20: mol_type = unassigned RNA
cttaaacaaacctggcttga

SEQ ID NO: 296 - DNA, 20 bp, synthetic construct
  source 1..20: mol_type = other DNA
tcaagccaggtttgtttaag

SEQ ID NO: 297 - RNA, 20 nt, Homo sapiens
  source 1..20: mol_type = unassigned RNA
caataaaactacagcccagg

SEQ ID NO: 298 - DNA, 20 bp, synthetic construct
  source 1..20: mol_type = other DNA
cctgggctgtagttttattg

SEQ ID NO: 299 - RNA, 20 nt, Homo sapiens
  source 1..20: mol_type = unassigned RNA
aagtctgtgacaccagaacg

SEQ ID NO: 300 - DNA, 20 bp, synthetic construct
  source 1..20: mol_type = other DNA
cgttctggtgtcacagactt

SEQ ID NO: 301 - RNA, 20 nt, Homo sapiens
  source 1..20: mol_type = unassigned RNA
gttgagctgtacctcccaca

SEQ ID NO: 302 - DNA, 20 bp, synthetic construct
  source 1..20: mol_type = other DNA
tgtgggaggtacagctcaac

SEQ ID NO: 303 - RNA, 20 nt, Homo sapiens
  source 1..20: mol_type = unassigned RNA
tacccttttctgaccaaaga

SEQ ID NO: 304 - DNA, 20 bp, synthetic construct
  source 1..20: mol_type = other DNA
tctttggtcagaaaagggta

SEQ ID NO: 305 - RNA, 20 nt, Homo sapiens
  source 1..20: mol_type = unassigned RNA
acccatatctcactcaggag

SEQ ID NO: 306 - DNA, 20 bp, synthetic construct
  source 1..20: mol_type = other DNA
ctcctgagtgagatatgggt

SEQ ID NO: 307 - RNA, 20 nt, Homo sapiens
  source 1..20: mol_type = unassigned RNA
atccccaagtctgtgacacc

SEQ ID NO: 308 - DNA, 20 bp, synthetic construct
  source 1..20: mol_type = other DNA
ggtgtcacagacttggggat

SEQ ID NO: 309 - RNA, 20 nt, Homo sapiens
  source 1..20: mol_type = unassigned RNA
tccccaagtctgtgacacca

SEQ ID NO: 310 - DNA, 20 bp, synthetic construct
  source 1..20: mol_type = other DNA
tggtgtcacagacttgggga

SEQ ID NO: 311 - RNA, 20 nt, Homo sapiens
  source 1..20: mol_type = unassigned RNA
caaggattaccccttccatg

SEQ ID NO: 312 - DNA, 20 bp, synthetic construct
  source 1..20: mol_type = other DNA
catggaaggggtaatccttg

SEQ ID NO: 313 - RNA, 20 nt, Homo sapiens
  source 1..20: mol_type = unassigned RNA
aaggattaccccttccatga

SEQ ID NO: 314 - DNA, 20 bp, synthetic construct
  source 1..20: mol_type = other DNA
tcatggaaggggtaatcctt

SEQ ID NO: 315 - RNA, 20 nt, Homo sapiens
  source 1..20: mol_type = unassigned RNA
aatagtgccactaacggttg

SEQ ID NO: 316 - DNA, 20 bp, synthetic construct
  source 1..20: mol_type = other DNA
caaccgttagtggcactatt

SEQ ID NO: 317 - RNA, 20 nt, Homo sapiens
  source 1..20: mol_type = unassigned RNA
ttggatgagtcgggcaatgt

SEQ ID NO: 318 - DNA, 20 bp, synthetic construct
  source 1..20: mol_type = other DNA
acattgcccgactcatccaa

SEQ ID NO: 319 - RNA, 20 nt, Homo sapiens
  source 1..20: mol_type = unassigned RNA
caacttcaaccatctccagg

SEQ ID NO: 320 - DNA, 20 bp, synthetic construct
  source 1..20: mol_type = other DNA
cctggagatggttgaagttg

SEQ ID NO: 321 - RNA, 20 nt, Homo sapiens
  source 1..20: mol_type = unassigned RNA
ataagcctgcagttaaccag

SEQ ID NO: 322 - DNA, 20 bp, synthetic construct
  source 1..20: mol_type = other DNA
ctggttaactgcaggcttat

SEQ ID NO: 323 - RNA, 20 nt, Homo sapiens
  source 1..20: mol_type = unassigned RNA
cctgcagttaaccagattga

SEQ ID NO: 324 - DNA, 20 bp, synthetic construct
  source 1..20: mol_type = other DNA
tcaatctggttaactgcagg

SEQ ID NO: 325 - RNA, 20 nt, Homo sapiens
  source 1..20: mol_type = unassigned RNA
gatccccaagtctgtgacac

SEQ ID NO: 326 - DNA, 20 bp, synthetic construct
  source 1..20: mol_type = other DNA
gtgtcacagacttggggatc

SEQ ID NO: 327 - RNA, 20 nt, Homo sapiens
  source 1..20: mol_type = unassigned RNA
tgttgagctgtacctcccac

SEQ ID NO: 328 - DNA, 20 bp, synthetic construct
  source 1..20: mol_type = other DNA
gtgggaggtacagctcaaca

SEQ ID NO: 329 - RNA, 20 nt, Homo sapiens
  source 1..20: mol_type = unassigned RNA
cactcagcagtgggacagca

SEQ ID NO: 330 - DNA, 20 bp, synthetic construct
  source 1..20: mol_type = other DNA
tgctgtcccactgctgagtg

SEQ ID NO: 331 - RNA, 20 nt, Homo sapiens
  source 1..20: mol_type = unassigned RNA
tggatgagtcgggcaatgtg

SEQ ID NO: 332 - DNA, 20 bp, synthetic construct
  source 1..20: mol_type = other DNA
cacattgcccgactcatcca

SEQ ID NO: 333 - RNA, 20 nt, Homo sapiens
  source 1..20: mol_type = unassigned RNA
aatgtggttcccagtgacac

SEQ ID NO: 334 - DNA, 20 bp, synthetic construct
  source 1..20: mol_type = other DNA
gtgtcactgggaaccacatt

SEQ ID NO: 335 - RNA, 20 nt, Homo sapiens
  source 1..20: mol_type = unassigned RNA
gatcaaggcgatcgcagcca

SEQ ID NO: 336 - DNA, 20 bp, synthetic construct
  source 1..20: mol_type = other DNA
tggctgcgatcgccttgatc

SEQ ID NO: 337 - RNA, 20 nt, Homo sapiens
  source 1..20: mol_type = unassigned RNA
tcgcagccaagcacaataaa

SEQ ID NO: 338 - DNA, 20 bp, synthetic construct
  source 1..20: mol_type = other DNA
tttattgtgcttggctgcga

SEQ ID NO: 339 - RNA, 20 nt, Homo sapiens
  source 1..20: mol_type = unassigned RNA
cgcagccaagcacaataaaa

SEQ ID NO: 340 - DNA, 20 bp, synthetic construct
  source 1..20: mol_type = other DNA
ttttattgtgcttggctgcg

SEQ ID NO: 341 - RNA, 20 nt, Homo sapiens
  source 1..20: mol_type = unassigned RNA
gcccttctttctaccctgct

SEQ ID NO: 342 - DNA, 20 bp, synthetic construct
  source 1..20: mol_type = other DNA
agcagggtagaaagaagggc

SEQ ID NO: 343 - RNA, 20 nt, Homo sapiens
  source 1..20: mol_type = unassigned RNA
gacctcacgggctatttaaa

SEQ ID NO: 344 - DNA, 20 bp, synthetic construct
  source 1..20: mol_type = other DNA
tttaaatagcccgtgaggtc

SEQ ID NO: 345 - RNA, 20 nt, Homo sapiens
  source 1..20: mol_type = unassigned RNA
tacctcccacaaggattacc

SEQ ID NO: 346 - DNA, 20 bp, synthetic construct
  source 1..20: mol_type = other DNA
ggtaatccttgtgggaggta

SEQ ID NO: 347 - RNA, 20 nt, Homo sapiens
  source 1..20: mol_type = unassigned RNA
cccttctttctaccctgctg

SEQ ID NO: 348 - DNA, 20 bp, synthetic construct
  source 1..20: mol_type = other DNA
cagcagggtagaaagaaggg

SEQ ID NO: 349 - RNA, 20 nt, Homo sapiens
  source 1..20: mol_type = unassigned RNA
acctcccacaaggattaccc

SEQ ID NO: 350 - DNA, 20 bp, synthetic construct
  source 1..20: mol_type = other DNA
gggtaatccttgtgggaggt

SEQ ID NO: 351 - RNA, 20 nt, Homo sapiens
  source 1..20: mol_type = unassigned RNA
acctcacgggctatttaaag

SEQ ID NO: 352 - DNA, 20 bp, synthetic construct
  source 1..20: mol_type = other DNA
ctttaaatagcccgtgaggt

SEQ ID NO: 353 - RNA, 20 nt, Homo sapiens
  source 1..20: mol_type = unassigned RNA
attgagtgccacccatatct

SEQ ID NO: 354 - DNA, 20 bp, synthetic construct
  source 1..20: mol_type = other DNA
agatatgggtggcactcaat

SEQ ID NO: 355 - RNA, 20 nt, Homo sapiens
  source 1..20: mol_type = unassigned RNA
gatcttaaacaaacctggct

SEQ ID NO: 356 - DNA, 20 bp, synthetic construct
  source 1..20: mol_type = other DNA
agccaggtttgtttaagatc

SEQ ID NO: 357 - RNA, 20 nt, Homo sapiens
  source 1..20: mol_type = unassigned RNA
tgatcttaaacaaacctggc

SEQ ID NO: 358 - DNA, 20 bp, synthetic construct
  source 1..20: mol_type = other DNA
gccaggtttgtttaagatca

SEQ ID NO: 359 - RNA, 20 nt, Homo sapiens
  source 1..20: mol_type = unassigned RNA
aaggcgatcgcagccaagca

SEQ ID NO: 360 - DNA, 20 bp, synthetic construct
  source 1..20: mol_type = other DNA
tgcttggctgcgatcgcctt

SEQ ID NO: 361 - RNA, 20 nt, Homo sapiens
  source 1..20: mol_type = unassigned RNA
gattgagtgccacccatatc

SEQ ID NO: 362 - DNA, 20 bp, synthetic construct
  source 1..20: mol_type = other DNA
gatatgggtggcactcaatc

SEQ ID NO: 363 - RNA, 20 nt, Homo sapiens
  source 1..20: mol_type = unassigned RNA
cagattgagtgccacccata

SEQ ID NO: 364 - DNA, 20 bp, synthetic construct
  source 1..20: mol_type = other DNA
tatgggtggcactcaatctg

SEQ ID NO: 365 - RNA, 20 nt, Homo sapiens
  source 1..20: mol_type = unassigned RNA
ccagattgagtgccacccat

SEQ ID NO: 366 - DNA, 20 bp, synthetic construct
  source 1..20: mol_type = other DNA
atgggtggcactcaatctgg

SEQ ID NO: 367 - RNA, 20 nt, Homo sapiens
  source 1..20: mol_type = unassigned RNA
tcaaggcgatcgcagccaag

SEQ ID NO: 368 - DNA, 20 bp, synthetic construct
  source 1..20: mol_type = other DNA
cttggctgcgatcgccttga

SEQ ID NO: 369 - RNA, 20 nt, Homo sapiens
  source 1..20: mol_type = unassigned RNA
taaacaaacctggcttgaag

SEQ ID NO: 370 - DNA, 20 bp, synthetic construct
  source 1..20: mol_type = other DNA
cttcaagccaggtttgttta

SEQ ID NO: 371 - RNA, 20 nt, Homo sapiens
  source 1..20: mol_type = unassigned RNA
ttaccccttccatgaagagt

SEQ ID NO: 372 - DNA, 20 bp, synthetic construct
  source 1..20: mol_type = other DNA
actcttcatggaaggggtaa

SEQ ID NO: 373 - RNA, 20 nt, Homo sapiens
  source 1..20: mol_type = unassigned RNA
aaacaaacctggcttgaagt

SEQ ID NO: 374 - DNA, 20 bp, synthetic construct
  source 1..20: mol_type = other DNA
acttcaagccaggtttgttt

SEQ ID NO: 375 - RNA, 20 nt, Homo sapiens
  source 1..20: mol_type = unassigned RNA
taccccttccatgaagagtt

SEQ ID NO: 376 - DNA, 20 bp, synthetic construct
  source 1..20: mol_type = other DNA
aactcttcatggaaggggta

SEQ ID NO: 377 - RNA, 20 nt, Homo sapiens
  source 1..20: mol_type = unassigned RNA
aatacaaaaattagcccggc

SEQ ID NO: 378 - DNA, 20 bp, synthetic construct
  source 1..20: mol_type = other DNA
gccgggctaatttttgtatt

SEQ ID NO: 379 - RNA, 20 nt, Homo sapiens
  source 1..20: mol_type = unassigned RNA
aataccatctcctcttcggc

SEQ ID NO: 380 - DNA, 20 bp, synthetic construct
  source 1..20: mol_type = other DNA
gccgaagaggagatggtatt

SEQ ID NO: 381 - RNA, 20 nt, Homo sapiens
  source 1..20: mol_type = unassigned RNA
aaataccatctcctcttcgg

SEQ ID NO: 382 - DNA, 20 bp, synthetic construct
  source 1..20: mol_type = other DNA
ccgaagaggagatggtattt

SEQ ID NO: 383 - RNA, 20 nt, Homo sapiens
  source 1..20: mol_type = unassigned RNA
ataccatctcctcttcggct

SEQ ID NO: 384 - DNA, 20 bp, synthetic construct
  source 1..20: mol_type = other DNA
agccgaagaggagatggtat

SEQ ID NO: 385 - RNA, 20 nt, Homo sapiens
  source 1..20: mol_type = unassigned RNA
cactacccttattcttgtct

SEQ ID NO: 386 - DNA, 20 bp, synthetic construct
  source 1..20: mol_type = other DNA
agacaagaataagggtagtg

SEQ ID NO: 387 - RNA, 20 nt, Homo sapiens
  source 1..20: mol_type = unassigned RNA
atacaaaaattagcccggca

SEQ ID NO: 388 - DNA, 20 bp, synthetic construct
  source 1..20: mol_type = other DNA
tgccgggctaatttttgtat

SEQ ID NO: 389 - RNA, 20 nt, Homo sapiens
  source 1..20: mol_type = unassigned RNA
tcactacccttattcttgtc

SEQ ID NO: 390 - DNA, 20 bp, synthetic construct
  source 1..20: mol_type = other DNA
gacaagaataagggtagtga

SEQ ID NO: 391 - RNA, 20 nt, Homo sapiens
  source 1..20: mol_type = unassigned RNA
taccatctcctcttcggctg

SEQ ID NO: 392 - DNA, 20 bp, synthetic construct
  source 1..20: mol_type = other DNA
cagccgaagaggagatggta

SEQ ID NO: 393 - RNA, 20 nt, Homo sapiens
  source 1..20: mol_type = unassigned RNA
acacccattcagtcattttg

SEQ ID NO: 394 - DNA, 20 bp, synthetic construct
  source 1..20: mol_type = other DNA
caaaatgactgaatgggtgt

SEQ ID NO: 395 - RNA, 20 nt, Homo sapiens
  source 1..20: mol_type = unassigned RNA
gacacccattcagtcatttt

SEQ ID NO: 396 - DNA, 20 bp, synthetic construct
  source 1..20: mol_type = other DNA
aaaatgactgaatgggtgtc

SEQ ID NO: 397 - RNA, 20 nt, Homo sapiens
  source 1..20: mol_type = unassigned RNA
tcatgataatctcccagaac

SEQ ID NO: 398 - DNA, 20 bp, synthetic construct
  source 1..20: mol_type = other DNA
gttctgggagattatcatga

SEQ ID NO: 399 - RNA, 20 nt, Homo sapiens
  source 1..20: mol_type = unassigned RNA
cagtttatcttatcgctcct

SEQ ID NO: 400 - DNA, 20 bp, synthetic construct
  source 1..20: mol_type = other DNA
aggagcgataagataaactg

SEQ ID NO: 401 - RNA, 20 nt, Homo sapiens
  source 1..20: mol_type = unassigned RNA
agtttatcttatcgctcctc

SEQ ID NO: 402 - DNA, 20 bp, synthetic construct
  source 1..20: mol_type = other DNA
gaggagcgataagataaact

SEQ ID NO: 403 - RNA, 20 nt, Homo sapiens
  source 1..20: mol_type = unassigned RNA
acctcccacaaggattaccc

SEQ ID NO: 404 - DNA, 20 bp, synthetic construct
  source 1..20: mol_type = other DNA
gggtaatccttgtgggaggt

SEQ ID NO: 405 - RNA, 20 nt, Homo sapiens
  source 1..20: mol_type = unassigned RNA
cctcccacaaggattacccc

SEQ ID NO: 406 - DNA, 20 bp, synthetic construct
  source 1..20: mol_type = other DNA
ggggtaatccttgtgggagg

SEQ ID NO: 407 - RNA, 20 nt, Homo sapiens
  source 1..20: mol_type = unassigned RNA
catgaggtcagaagatagag

SEQ ID NO: 408 - DNA, 20 bp, synthetic construct
  source 1..20: mol_type = other DNA
ctctatcttctgacctcatg

SEQ ID NO: 409 - RNA, 20 nt, Homo sapiens
  source 1..20: mol_type = unassigned RNA
cttcatgataatctcccaga

SEQ ID NO: 410 - DNA, 20 bp, synthetic construct
  source 1..20: mol_type = other DNA
tctgggagattatcatgaag

SEQ ID NO: 411 - RNA, 20 nt, Homo sapiens
  source 1..20: mol_type = unassigned RNA
atgaggtcagaagatagaga

SEQ ID NO: 412 - DNA, 20 bp, synthetic construct
  source 1..20: mol_type = other DNA
tctctatcttctgacctcat

SEQ ID NO: 413 - RNA, 20 nt, Homo sapiens
  source 1..20: mol_type = unassigned RNA
actgttttctcctttctcct

SEQ ID NO: 414 - DNA, 20 bp, synthetic construct
  source 1..20: mol_type = other DNA
aggagaaaggagaaaacagt

SEQ ID NO: 415 - RNA, 20 nt, Homo sapiens
  source 1..20: mol_type = unassigned RNA
ttctcctttctcctgacagc

SEQ ID NO: 416 - DNA, 20 bp, synthetic construct
  source 1..20: mol_type = other DNA
gctgtcaggagaaaggagaa

SEQ ID NO: 417 - RNA, 20 nt, Homo sapiens
  source 1..20: mol_type = unassigned RNA
agacccagcctcctttccct

SEQ ID NO: 418 - DNA, 20 bp, synthetic construct
  source 1..20: mol_type = other DNA
agggaaaggaggctgggtct

SEQ ID NO: 419 - RNA, 20 nt, Homo sapiens
  source 1..20: mol_type = unassigned RNA
cccttattcttgtctgagct

SEQ ID NO: 420 - DNA, 20 bp, synthetic construct
  source 1..20: mol_type = other DNA
agctcagacaagaataaggg

SEQ ID NO: 421 - RNA, 20 nt, Homo sapiens
  source 1..20: mol_type = unassigned RNA
ctctcccattcatccgtctt

SEQ ID NO: 422 - DNA, 20 bp, synthetic construct
  source 1..20: mol_type = other DNA
aagacggatgaatgggagag

SEQ ID NO: 423 - RNA, 20 nt, Homo sapiens
  source 1..20: mol_type = unassigned RNA
catgataatctcccagaacc

SEQ ID NO: 424 - DNA, 20 bp, synthetic construct
  source 1..20: mol_type = other DNA
ggttctgggagattatcatg

SEQ ID NO: 425 - RNA, 20 nt, Homo sapiens
  source 1..20: mol_type = unassigned RNA
tctctcccattcatccgtct

SEQ ID NO: 426 - DNA, 20 bp, synthetic construct
  source 1..20: mol_type = other DNA
agacggatgaatgggagaga

SEQ ID NO: 427 - RNA, 20 nt, Homo sapiens
  source 1..20: mol_type = unassigned RNA
ccttattcttgtctgagctc

SEQ ID NO: 428 - DNA, 20 bp, synthetic construct
  source 1..20: mol_type = other DNA
gagctcagacaagaataagg

SEQ ID NO: 429 - RNA, 20 nt, Homo sapiens
  source 1..20: mol_type = unassigned RNA
tctctcactacccttattct

SEQ ID NO: 430 - DNA, 20 bp, synthetic construct
  source 1..20: mol_type = other DNA
agaataagggtagtgagaga

SEQ ID NO: 431 - RNA, 20 nt, Homo sapiens
  source 1..20: mol_type = unassigned RNA
cctttcttgccctctctgtc

SEQ ID NO: 432 - DNA, 20 bp, synthetic construct
  source 1..20: mol_type = other DNA
gacagagagggcaagaaagg

SEQ ID NO: 433 - RNA, 20 nt, Homo sapiens
  source 1..20: mol_type = unassigned RNA
ccaatctgtccattccattt

SEQ ID NO: 434 - DNA, 20 bp, synthetic construct
  source 1..20: mol_type = other DNA
aaatggaatggacagattgg

SEQ ID NO: 435 - RNA, 20 nt, Homo sapiens
  source 1..20: mol_type = unassigned RNA
ctctctcctttagtacttcc

SEQ ID NO: 436 - DNA, 20 bp, synthetic construct
  source 1..20: mol_type = other DNA
ggaagtactaaaggagagag

SEQ ID NO: 437 - RNA, 20 nt, Homo sapiens
  source 1..20: mol_type = unassigned RNA
tctctcctttagtacttcct

SEQ ID NO: 438 - DNA, 20 bp, synthetic construct
  source 1..20: mol_type = other DNA
aggaagtactaaaggagaga

SEQ ID NO: 439 - RNA, 20 nt, Homo sapiens
  source 1..20: mol_type = unassigned RNA
cctgagcctgattatctgaa

SEQ ID NO: 440 - DNA, 20 bp, synthetic construct
  source 1..20: mol_type = other DNA
ttcagataatcaggctcagg

SEQ ID NO: 441 - RNA, 20 nt, Homo sapiens
  source 1..20: mol_type = unassigned RNA
ccatcttttgccatgatcac

SEQ ID NO: 442 - DNA, 20 bp, synthetic construct
  source 1..20: mol_type = other DNA
gtgatcatggcaaaagatgg

SEQ ID NO: 443 - RNA, 20 nt, Homo sapiens
  source 1..20: mol_type = unassigned RNA
tttctccatcttttgccatg

SEQ ID NO: 444 - DNA, 20 bp, synthetic construct
  source 1..20: mol_type = other DNA
catggcaaaagatggagaaa

SEQ ID NO: 445 - RNA, 20 nt, Homo sapiens
  source 1..20: mol_type = unassigned RNA
gccaatctgtccattccatt

SEQ ID NO: 446 - DNA, 20 bp, synthetic construct
  source 1..20: mol_type = other DNA
aatggaatggacagattggc

SEQ ID NO: 447 - RNA, 20 nt, Homo sapiens
  source 1..20: mol_type = unassigned RNA
tccatcttttgccatgatca

SEQ ID NO: 448 - DNA, 20 bp, synthetic construct
  source 1..20: mol_type = other DNA
tgatcatggcaaaagatgga

SEQ ID NO: 449 - RNA, 20 nt, Homo sapiens
  source 1..20: mol_type = unassigned RNA
ctgagcctgattatctgaaa

SEQ ID NO: 450 - DNA, 20 bp, synthetic construct
  source 1..20: mol_type = other DNA
tttcagataatcaggctcag

SEQ ID NO: 451 - RNA, 20 nt, Homo sapiens
  source 1..20: mol_type = unassigned RNA
cttcttcgtgacctcttgca

SEQ ID NO: 452 - DNA, 20 bp, synthetic construct
  source 1..20: mol_type = other DNA
tgcaagaggtcacgaagaag

SEQ ID NO: 453 - RNA, 20 nt, Homo sapiens
  source 1..20: mol_type = unassigned RNA
attgacttccccaaacaaca

SEQ ID NO: 454 - DNA, 20 bp, synthetic construct
  source 1..20: mol_type = other DNA
tgttgtttggggaagtcaat

SEQ ID NO: 455 - RNA, 20 nt, Homo sapiens
  source 1..20: mol_type = unassigned RNA
ctagaaaaacctctcacaac

SEQ ID NO: 456 - DNA, 20 bp, synthetic construct
  source 1..20: mol_type = other DNA
gttgtgagaggtttttctag

SEQ ID NO: 457 - RNA, 20 nt, Homo sapiens
  source 1..20: mol_type = unassigned RNA
ttgacttccccaaacaacag

SEQ ID NO: 458 - DNA, 20 bp, synthetic construct
  source 1..20: mol_type = other DNA
ctgttgtttggggaagtcaa

SEQ ID NO: 459 - RNA, 20 nt, Homo sapiens
  source 1..20: mol_type = unassigned RNA
tagcctgagcctgattatct

SEQ ID NO: 460 - DNA, 20 bp, synthetic construct
  source 1..20: mol_type = other DNA
agataatcaggctcaggcta

SEQ ID NO: 461 - RNA, 20 nt, Homo sapiens
  source 1..20: mol_type = unassigned RNA
aacgtatcacccacggccat

SEQ ID NO: 462 - DNA, 20 bp, synthetic construct
  source 1..20: mol_type = other DNA
atggccgtgggtgatacgtt

SEQ ID NO: 463 - RNA, 20 nt, Homo sapiens
  source 1..20: mol_type = unassigned RNA
acactcaatacagctacact

SEQ ID NO: 464 - DNA, 20 bp, synthetic construct
  source 1..20: mol_type = other DNA
agtgtagctgtattgagtgt

SEQ ID NO: 465 - RNA, 20 nt, Homo sapiens
  source 1..20: mol_type = unassigned RNA
gcttcttcgtgacctcttgc

SEQ ID NO: 466 - DNA, 20 bp, synthetic construct
  source 1..20: mol_type = other DNA
gcaagaggtcacgaagaagc

SEQ ID NO: 467 - RNA, 20 nt, Homo sapiens
  source 1..20: mol_type = unassigned RNA
atagcctgagcctgattatc

SEQ ID NO: 468 - DNA, 20 bp, synthetic construct
  source 1..20: mol_type = other DNA
gataatcaggctcaggctat

SEQ ID NO: 469 - RNA, 20 nt, Homo sapiens
  source 1..20: mol_type = unassigned RNA
gtaactagcaagacctgtaa

SEQ ID NO: 470 - DNA, 20 bp, synthetic construct
  source 1..20: mol_type = other DNA
ttacaggtcttgctagttac

SEQ ID NO: 471 - RNA, 20 nt, Homo sapiens
  source 1..20: mol_type = unassigned RNA
ataagcatctcaagccggta

SEQ ID NO: 472 - DNA, 20 bp, synthetic construct
  source 1..20: mol_type = other DNA
taccggcttgagatgcttat

SEQ ID NO: 473 - RNA, 20 nt, Homo sapiens
  source 1..20: mol_type = unassigned RNA
aaacgtatcacccacggcca

SEQ ID NO: 474 - DNA, 20 bp, synthetic construct
  source 1..20: mol_type = other DNA
tggccgtgggtgatacgttt

SEQ ID NO: 475 - RNA, 20 nt, Homo sapiens
  source 1..20: mol_type = unassigned RNA
ccgtagctagttgataattc

SEQ ID NO: 476 - DNA, 20 bp, synthetic construct
  source 1..20: mol_type = other DNA
gaattatcaactagctacgg

SEQ ID NO: 477 - RNA, 20 nt, Homo sapiens
  source 1..20: mol_type = unassigned RNA
atttgtttctaccccagctc

SEQ ID NO: 478 - DNA, 20 bp, synthetic construct
  source 1..20: mol_type = other DNA
gagctggggtagaaacaaat

SEQ ID NO: 479 - RNA, 20 nt, Homo sapiens
  source 1..20: mol_type = unassigned RNA
aataagcatctcaagccggt

SEQ ID NO: 480 - DNA, 20 bp, synthetic construct
  source 1..20: mol_type = other DNA
accggcttgagatgcttatt

SEQ ID NO: 481 - RNA, 20 nt, Homo sapiens
  source 1..20: mol_type = unassigned RNA
tttgtttctaccccagctct

SEQ ID NO: 482 - DNA, 20 bp, synthetic construct
  source 1..20: mol_type = other DNA
agagctggggtagaaacaaa

SEQ ID NO: 483 - RNA, 20 nt, Homo sapiens
  source 1..20: mol_type = unassigned RNA
acgtatcacccacggccata

SEQ ID NO: 484 - DNA, 20 bp, synthetic construct
  source 1..20: mol_type = other DNA
tatggccgtgggtgatacgt

SEQ ID NO: 485 - RNA, 20 nt, Homo sapiens
  source 1..20: mol_type = unassigned RNA
taaaacctcaacaccacaca

SEQ ID NO: 486 - DNA, 20 bp, synthetic construct
  source 1..20: mol_type = other DNA
tgtgtggtgttgaggtttta

SEQ ID NO: 487 - RNA, 20 nt, Homo sapiens
  source 1..20: mol_type = unassigned RNA
gtcccctcttaacccacacc

SEQ ID NO: 488 - DNA, 20 bp, synthetic construct
  source 1..20: mol_type = other DNA
ggtgtgggttaagaggggac

SEQ ID NO: 489 - RNA, 20 nt, Homo sapiens
  source 1..20: mol_type = unassigned RNA
gtctcagctccagcccgttt

SEQ ID NO: 490 - DNA, 20 bp, synthetic construct
  source 1..20: mol_type = other DNA
aaacgggctggagctgagac

SEQ ID NO: 491 - RNA, 20 nt, Homo sapiens
  source 1..20: mol_type = unassigned RNA
agtctcagctccagcccgtt

SEQ ID NO: 492 - DNA, 20 bp, synthetic construct
  source 1..20: mol_type = other DNA
aacgggctggagctgagact

SEQ ID NO: 493 - RNA, 20 nt, Homo sapiens
  source 1..20: mol_type = unassigned RNA
aaaacctcaacaccacacag

SEQ ID NO: 494 - DNA, 20 bp, synthetic construct
  source 1..20: mol_type = other DNA
ctgtgtggtgttgaggtttt

SEQ ID NO: 495 - RNA, 20 nt, Homo sapiens
  source 1..20: mol_type = unassigned RNA
cccgtagctagttgataatt

SEQ ID NO: 496 - DNA, 20 bp, synthetic construct
  source 1..20: mol_type = other DNA
aattatcaactagctacggg

SEQ ID NO: 497 - RNA, 20 nt, Homo sapiens
  source 1..20: mol_type = unassigned RNA
tagtgtccatgtctccgtct

SEQ ID NO: 498 - DNA, 20 bp, synthetic construct
  source 1..20: mol_type = other DNA
agacggagacatggacacta

SEQ ID NO: 499 - RNA, 20 nt, Homo sapiens
  source 1..20: mol_type = unassigned RNA
ctcacaaccattcacagcat

SEQ ID NO: 500 - DNA, 20 bp, synthetic construct
  source 1..20: mol_type = other DNA
atgctgtgaatggttgtgag

SEQ ID NO: 501 - RNA, 20 nt, Homo sapiens
  source 1..20: mol_type = unassigned RNA
acaaccattcacagcatcat

SEQ ID NO: 502 - DNA, 20 bp, synthetic construct
  source 1..20: mol_type = other DNA
atgatgctgtgaatggttgt

SEQ ID NO: 503 - RNA, 20 nt, Homo sapiens
  source 1..20: mol_type = unassigned RNA
gcattttgttattctgtccc

SEQ ID NO: 504 - DNA, 20 bp, synthetic construct
  source 1..20: mol_type = other DNA
gggacagaataacaaaatgc

SEQ ID NO: 505 - RNA, 20 nt, Homo sapiens
  source 1..20: mol_type = unassigned RNA
tccgtctgacagtctcagct

SEQ ID NO: 506 - DNA, 20 bp, synthetic construct
  source 1..20: mol_type = other DNA
agctgagactgtcagacgga

SEQ ID NO: 507 - RNA, 20 nt, Homo sapiens
  source 1..20: mol_type = unassigned RNA
cattccattttgtgtcctct

SEQ ID NO: 508 - DNA, 20 bp, synthetic construct
  source 1..20: mol_type = other DNA
agaggacacaaaatggaatg

SEQ ID NO: 509 - RNA, 20 nt, Homo sapiens
  source 1..20: mol_type = unassigned RNA
ttagagtcccgtgctgatga

SEQ ID NO: 510 - DNA, 20 bp, synthetic construct
  source 1..20: mol_type = other DNA
tcatcagcacgggactctaa

SEQ ID NO: 511 - RNA, 20 nt, Homo sapiens
  source 1..20: mol_type = unassigned RNA
gttttattgcaccacagcat

SEQ ID NO: 512 - DNA, 20 bp, synthetic construct
  source 1..20: mol_type = other DNA
atgctgtggtgcaataaaac

SEQ ID NO: 513 - RNA, 20 nt, Homo sapiens
  source 1..20: mol_type = unassigned RNA
taagcatctcaagccggtag

SEQ ID NO: 514 - DNA, 20 bp, synthetic construct
  source 1..20: mol_type = other DNA
ctaccggcttgagatgctta

SEQ ID NO: 515 - RNA, 20 nt, Homo sapiens
  source 1..20: mol_type = unassigned RNA
tgccctcacaaccattcaca

SEQ ID NO: 516 - DNA, 20 bp, synthetic construct
  source 1..20: mol_type = other DNA
tgtgaatggttgtgagggca

SEQ ID NO: 517 - RNA, 20 nt, Homo sapiens
  source 1..20: mol_type = unassigned RNA
tatcacccacggccatacag

SEQ ID NO: 518 - DNA, 20 bp, synthetic construct
  source 1..20: mol_type = other DNA
ctgtatggccgtgggtgata

SEQ ID NO: 519 - RNA, 20 nt, Homo sapiens
  source 1..20: mol_type = unassigned RNA
atgacaccaagctaatttcc

SEQ ID NO: 520 - DNA, 20 bp, synthetic construct
  source 1..20: mol_type = other DNA
ggaaattagcttggtgtcat

SEQ ID NO: 521 - RNA, 20 nt, Homo sapiens
  source 1..20: mol_type = unassigned RNA
ttattgcaccacagcatatc

SEQ ID NO: 522 - DNA, 20 bp, synthetic construct
  source 1..20: mol_type = other DNA
gatatgctgtggtgcaataa

SEQ ID NO: 523 - RNA, 20 nt, Homo sapiens
  source 1..20: mol_type = unassigned RNA
gcatgggctcttagattaat

SEQ ID NO: 524 - DNA, 20 bp, synthetic construct
  source 1..20: mol_type = other DNA
attaatctaagagcccatgc

SEQ ID NO: 525 - RNA, 20 nt, Homo sapiens
  source 1..20: mol_type = unassigned RNA
attccattttgtgtcctctg

SEQ ID NO: 526 - DNA, 20 bp, synthetic construct
  source 1..20: mol_type = other DNA
cagaggacacaaaatggaat

SEQ ID NO: 527 - RNA, 20 nt, Homo sapiens
  source 1..20: mol_type = unassigned RNA
ggctaatcttttttcttccc

SEQ ID NO: 528 - DNA, 20 bp, synthetic construct
  source 1..20: mol_type = other DNA
gggaagaaaaaagattagcc

SEQ ID NO: 529 - RNA, 20 nt, Homo sapiens
  source 1..20: mol_type = unassigned RNA
acagatgctcgacagaagat

SEQ ID NO: 530 - DNA, 20 bp, synthetic construct
  source 1..20: mol_type = other DNA
atcttctgtcgagcatctgt

SEQ ID NO: 531 - RNA, 20 nt, Homo sapiens
  source 1..20: mol_type = unassigned RNA
gatgctcgacagaagatgtg

SEQ ID NO: 532 - DNA, 20 bp, synthetic construct
  source 1..20: mol_type = other DNA
cacatcttctgtcgagcatc

SEQ ID NO: 533 - RNA, 20 nt, Homo sapiens
  source 1..20: mol_type = unassigned RNA
ttgtttcctccaaaaactcc

SEQ ID NO: 534 - DNA, 20 bp, synthetic construct
  source 1..20: mol_type = other DNA
ggagtttttggaggaaacaa

SEQ ID NO: 535 - RNA, 20 nt, Homo sapiens
  source 1..20: mol_type = unassigned RNA
gtacttaacctatgtaggct

SEQ ID NO: 536 - DNA, 20 bp, synthetic construct
  source 1..20: mol_type = other DNA
agcctacataggttaagtac

SEQ ID NO: 537 - protein, 7 aa, Homo sapiens
PNNLSLV

SEQ ID NO: 538 - protein, 7 aa, Macaca mulatta
PKNLSLV

SEQ ID NO: 539 - protein, 7 aa, Mus musculus
GENLSLV

SEQ ID NO: 540 - protein, 7 aa, unidentified
  source 1..7: note = Canis lupus familiaris
AENLTLV

SEQ ID NO: 541 - protein, 7 aa, Drosophila sp.
KDNLTAV

SEQ ID NO: 542 - protein, 7 aa, Homo sapiens
KMGRYNL

SEQ ID NO: 543 - protein, 7 aa, Homo sapiens
EEGALIE

SEQ ID NO: 544 - protein, 7 aa, Homo sapiens
KPLVTHR

SEQ ID NO: 545 - protein, 7 aa, Macaca mulatta
KSGRYNL

SEQ ID NO: 546 - protein, 7 aa, Macaca mulatta
EEGALIE

SEQ ID NO: 547 - protein, 7 aa, Macaca mulatta
KPLVTHR

SEQ ID NO: 548 - protein, 7 aa, Mus musculus
KIGRYNL

SEQ ID NO: 549 - protein, 7 aa, Mus musculus
EEGALIE

SEQ ID NO: 550 - protein, 7 aa, Mus musculus
KPLVTHR

SEQ ID NO: 551 - protein, 7 aa, unidentified
  source 1..7: note = Canis lupus familiaris
KIGRYNL

SEQ ID NO: 552 - protein, 7 aa, unidentified
  source 1..7: note = Canis lupus familiaris
EEGALIE

SEQ ID NO: 553 - protein, 7 aa, unidentified
  source 1..7: note = Canis lupus familiaris
KPLVTHR

SEQ ID NO: 554 - protein, 7 aa, Drosophila sp.
KQGRYNL

SEQ ID NO: 555 - protein, 7 aa, Drosophila sp.
EEGALLE

SEQ ID NO: 556 - protein, 7 aa, Drosophila sp.
KRLVTHH

SEQ ID NO: 557 - DNA, 16843 bp, Homo sapiens
  source 1..16843: mol_type = unassigned DNA
agaaggctccttcgcgcagcggcgcgccaaccgcaggcgccctttctgccgacctcacgggctatttaaaggtacgcgccgcggccaaggccgcaccgtactgggcgggggtctggggagcgcagcagccatggcaagccgtctcctgctcaacaacggcgccaagatgcccatcctggggttgggtacctggaaggtaggtgctcgtgggggcgcgggcccggggctcgcctcacactctccgcgcggcctgtattggcgagggaccccgagtgaccctgagcagctcgccccgcggacgcccggcgtgctgggagccacgcgcgggcttgcagggtccccagcgggctggggtcggccttgcagagaccgggggccttggctccccgggttggccctgggcgtcagggcagcatcctgcgagtggggtttgggagcagctcacgggagcccccgccctaccgcgggcaacccttgatgggcggcccaccagtccgcattttgggtcctagcgggcgccccaagcggcacaacgcgagagggaggcggggaaagtgggcttcacagaccggtggacctcgggcgcagacagggacgtggagccgtcggcaaggtgtgggagcgcagacccagcctcctttccctcgaggcacctgtagcccggttgcctcacttgtaaatagttggtctcaatcgtttcgattcttgccctccctgaggaataagcatctcaagccggtagagggcagagaattaggtggcgcgagtttgcccggacccagctgttaacaaggcgcagtgcggagctcctcggccaggcgccgcctcggggtgccctgggtctggaactcgggagaaaaatctgggcaccccgtagctagttgataattcaggaggcgccaaggttagcattttccctgtaaaacctcaacaccacacagcgtccccgagaagtgcctagccggatgagaatgacaataggacatgaaacatcataggatatgaaaaatgataatatgatatgaaaaaccaaaaataatagcctgagcctgattatctgaaaattgagcaacaaaggtggtggtatgggagctgaggcaaaacctagaatttttaaccttctaagtattgcatattatattccacagccataacctgggtttctcagtttctgtttttgttgtttgcttgtttgtttgtgacagggtctcgttgcccaagctggggtgcagtggcacaatcatggttcattgcagcctggaactcctgggctcaagtgatcctcctgcctcagcactgcgggtagctgggactacaggcgctcaccaccacacctggctaatcttttttcttccccgcagagacagtctccgcttgttgcttagggtgatctccaattcctgggctcaaagcgctcctcccacctcgcctcccaaagctacaggtgtgaaccatctggcaggcccacgtcccaggtttttctcttaaaatttgaatatgggcctgggcgttggatatttacacacagacaagggaagaaaacaacagaaagaactgagtgaactgtgagatgtttgaaataggacagaagagtttacatgtaccctaaaacctgctgttgaaaattagtaaatataaggtaagaaaaaggtcatttgcatcagggagagtgtttcttggccagaatgatcagggtacgtgatttgtttcctccaaaaactccagtggtgtctctaaggttgtaattgaattacaaccatgacaccaagctaatttccatctgttgcatgttatgctcttggaatggatgcagaaggatgctttcttggcaatagcaaatactttctaagcatgatgcaacgcacagatatatacatgtaactggaataaaaattacttaacagtacttaacctatgtaggctgcatttcaatatttttttctattccatttttccaaaaattattgtcatacctccagaagctagttttgtgacccatcatttgaaaatcactactctagggtaaataaggagtaaaagtttatttttttttttttgagacaggggctcagtgtcacccaggctggagtgcagtggcatgatcatgattcactgtagccttaacctcttgggctcaagtggtcctcatacctcagcctcccaagtagctgggaccacaggtgtgtgccaccacactcgggtagtttgtttttaattttttgtagagacaggtttccctatgttgtccaggctgaggagtaaaagtttatgaaagagggtttcagattattcacttgagcactcaatgccccaaaaagctatgtatttgtttaagataacatcacatcataacatctgtacatttaggttatgcattccagtagacagagttcattgagaaattggataatgtctgcttcattcattactatttctagcacttctcacagtgcctggcaaatataggagtaggtatccaaaagttatgtgttgaattcatatgaagaagtaaaacgactttagagatagctgtatggtgaaaacgcccacttgtattcctgtggatgaggagatcaggttggtgtagaattgtacggtgggggcagcggggaatggtgggtcaaagtaatggtataaggatggacttggaatcaacttcctttttttttttaatgacttaaaacctttccctggggccgggcgcggtggctcacacctgtaaccccagcactttgggaggcagaggcgggcagatcatgaggtcagaagatagagaccgtcctggctaacatggtgaaaccccgtctctactaaaaatacaaaaaattagcccggcatcgtggcacgcacctgtagtcccagctacttgggaggctgaggcaggaaaatcgcttgaaccccagatgtggaggttgcagtgagccgaggtcccgccactgcactccagcctgggcgatggagcaagactccatctaaaaaaacaaccaaacaaaaaaacctttcccttgtatgtgtgtatgtattctttcacagatgctcgacagaagatgtgatcctttgcctgtctttgtgacagctggaggaggccctgcaggcaggtgcagttccttattctcctgggtcttcccatgccgtagcggctgtgttgtacctttcagaccctcagcagggctctggtagtctctactcttgaaggtcctcaaactccattctcttctcaggtgagggtttcaggcagatggggagaccatctctgccccagacccatgtggcttggacagggtttctttctggcagtcttgccttctacattggatagtaaacaattacacctgtaatcccagcactttggaaggccaaggcgggtggatcacctgaggtcaggagttcgagaccagctggccaacatggtgaaaccccctctctactaaaaatacaaaaattagcccggcatggtggtgggtgcctgtaatcccagctgcttgggaggctgaggcaggagaatcacttgaatccagaaggtagaggttgccatgatctgagattgtgccactgcactccagcctaggcgacaagagcaaaaaaaaactcggtctcagaaacaacaacaacaacaaaaacccatcacagcattgtaaagccaggaagcttttaaaacagagatgatgtctggtgcccaccagtgtcttgttactgatagtgtttttaatctagttgaaaaattgttggaggtaaaagtcattgtaagtctcctatgtatgctcacagaatactcctagggctgaaggtacaactatgtttctccatcttttgccatgatcactacctccctcccacctaggagccttgtgagacttttttttcccctaatttgccccaaaataaaattgtaatatcagagataatattgtacttctgcttatggccagtagccttctagagggtcacaagctgttctataagctgaggggggtttttttgccccagagaactaatttttgtctctttggggacaatagcacccactttaagaatgcatgaggtaaaagaaaatgtgccaggaaagacttgctccaccttccccaagtggggtaagcttgtgatggcccaagatccttggatggggtgagggttgaggcgctgtgggcatggaggtaacacaggagccagatttactgttcacttccctggtgtctctaaagcccccatctcttcctcttcaagaccaaagacatttaaataagccctgcccctacagggtcagtcctggtccatgcacatctcaactagttggaaacagcagtgtcttagaatttgggacaaattgattaaaacagaacaggaggaagttaatttctgctgccaaacctgattctgagtgatcgtttcctgttccttgctccaggattatcagcacagcattctcatgcttcctactcaggaccgtttctttacccaaagtgaagtttcctttgccccctccccttatcctttgatttcagtagtaacacctgctaactagagaacatttcaaaaatgaagagttccctgttcccagagatccccgtggttaatattgcaaaatatccaatctctgagatactttggttttaccataaacactgtcctgtgaccgtcttcctgtttagcactgtggcttgctttttgacttgcagagggaagagagggaggggtatcttgaagtaactagcaagacctgtaagcaaaagggaagaagactaggttttaaaaggagtgttgtatctgtgtaggagggagacactgattttagttatcagtgtctcagagttggtattagccagccggtgttatcaaaggaggaactaggaccgctgtcttgaaggaggtggtggaggactcaagggcagaggcttctcactgccatcacttggtgccatcagcccagagcatttcaagtcactgcccaacgtagagctttgcagagccagaagttctgggaggaatgggcatgcatggttttgcccatcaagggcacacagattagtggcaggatcgggttagaaacatgtaggatttgtatgattgagctgtaattggagtgtttcatatactgcatttctacagctctcatcagtaatggctgtgcctggatgccacccagcatgttttcatagtttaccttctttttagaagtcaatctggacccctccttctgtttgcattttctctttgttgacatggaatggggagatgttgtcatcctgtgttggcattcccaagtttagtgaatggtttctcaccatagcttaagttggctgcttcatttttcataaggcaaattagtttctctcagggaagggctgtacattgagatcattaacatagaatgtggttgtggatcctcgtacattttcattgcagaggtgactttttccctccagcacctttgatcagcctcccagcacacatgtctccagcgaatggcacatatccaaggaatgatcagcagcttcttcgtgacctcttgcagtcattgctgtcttttacttttctagaaaaacctctcacaacttttttttcatgtcgtgccaacgcaagcttgattatcctagtctgtgctcttttggttccaaagaactgaaaaaccactttaatcttaagcaggaaaaaaaaaagtgtgtttggggtggggattgattgacttccccaaacaacagccgctgacattaggtgtttgagattcagggttgtaaatgttgtcaccaggatttggcctccctccactttcctgcagcagctccaggctgacattctcccagctcagtgaccccagcagaaccggggctggactcaggggttccaattcaagtgcaaagattgagtcttatggcctggattgagtcatgttcctgtccccgcaccagttgcatggcaggcagtgcgtggctaggctgagcctgcggagtgtgccctcagcagctcctcagagaaaaatcaatcaaggcgccatcaagcctggagaaagttcagtgtgtcctgggcagtttcctgcctgggccccagttgttctttctggggtagggatgtcctctgctctccctgctgtcatcagtggtggttgtgcccggacaccactcagggcctgtggtaaggcattctttctggaaattgagattctctgttccttcaaccgtgcagagtggggaaggctgcaagagcagcttctgaggtcctccagtcaaataagaggtcgccaggtcctctgagggtcgtcccctcttaacccacaccttcgatgagcatccccaccctgcctcgctgctccctctgaggtggagagtgagctccatgctaacaggctctggtaacgggcttgttcaactgggcccctttcttccccaaagcctgttgaaaatagctgatgtcaaaacacagatgtctgcagctgctatggccctggggggtttttcttttcatttgtttctaccccagctctccaaaggaatgtgctctgttcattcaacagcccgctgcacatgtttattgctcagctctgtgatcatagcaggatagtgaaagttgggggatacgtgtccatgcagcctacctgggtgccagtgaagcgcgatgccatgtaggcacttcctcctcctcttcagtagccaggctccccacagctgttctggttactcagatggtcaggtggcttggccatggacacccattcagtcattttgccaggtcagccactaaccatttggtgatcttccatgatccacattccccctcgtttccctttaacttatttctctctgctgatacctctgaggggccatctcccgtgtctccaccccttgaagcagatcctcatacagtgagaattgacacatgggcctttgaataaacatcagtgatgtgcttaacttcatgataatctcccagaaccgcttgaaaacaagcgactgccggtatcagaagccatgcatgaaaagagagcgtaaggggcaggcattcactggctgaactggggagccacatgggaattggggtccataggacatctatttttgaaatattttgagatatttgaaggaaatggtttcccttactatctcagcattcagagtgtggggaaatgtttttaatacagtggtttgcaaatcagtggatgtgaagaagcaagaaaatgagatggcttcagcatatcttttaaaagtgatctcacctacacttaagtggctgtcagattctcctacagggcaatgctgtgtggcctttcacttccatcccccgcgtctgaggctgatagtccagaatgctgctgaggaagtgcacgtggtctgtctttgctgaacaaagtggcaggctggtcagcaggtatcggtggctgccactgcccctcctccttgctgtgcagactctgcattctgcggggctcacgttctttctcctttcagtcccctccagggcaggtgactgaggccgtgaaggtggccattgacgtcgggtaccgccacatcgactgtgcccatgtgtaccagaatgagaatgaggtgggggtggccattcaggagaagctcagggagcaggtggtgaagcgtgaggagctcttcatcgtcagcaaggtatcgttccgcggtggggctggaaggggctctcggtcccatcatctgttggtgcatcagcccaaagccaagtccacactcaatacagctacacttttcactgatgggcgattcttcccggggaaacgtatcacccacggccatacagggctccagagctcgcccatccttggctctctgttgaatggtcattccatctgggcccattgcttagagtcccgtgctgatgaggctgaggttgtggattggccctccctttccagggagtcgctttcaaggatggaacttggttcccatctgcaaactgaatctccagtgctagccactgtctttgacctgagatactgatgcacaaaggacatgaggcaggaggatggggctggctccacaaacaccagcccggggggaaagtttcgagagttcttgcttaggcctagggcaggctagccaatctgtccattccattttgtgtcctctggtccttgtaagaggtctctgtgacctggtcatcctgcctgcctttggaaagttaggatccagtacgcagtggaaatgccctcacaaccattcacagcatcatggtaaagcaaatgggttttggggagaggagactgctctgtctttccagccaggaagctgccttgtggctggcaatgatgacatgtgccctctcgctggcttagctgtggtgcacgtaccatgagaagggcctggtgaaaggagcctgccagaagacactcagcgacctgaagctggactacctggacctctaccttattcactggccgactggctttaaggtatgggatgcctggtgcagaccaggtcccttggggctcaggtgcaacctgtggtgtgttttgagctcccttagcagattacgtgtgggtgactttctgttcttgctgctcaaggtatagccagtggaccatcagcactggcatcacctgggagcgtcttagaaatcacagtctcgggccccacgcaggccatttgaatccgcaccagcagtgactcatacgcacacttcttcaaagtttgaggagcactgctctttgccagttggtagatgtgcaatttaaaactgtcagcaaacacagctgctgtctgtagtgcacacagccatgaggcacggtcagccctggccctcccctccaggcacgtgcacacaccaacccccatacacatgagtccctacccacgttctacatgcataccctgtcctgtggaggcccggtaactatttgcagtctagttgttggcacaagaagaacttgagagttactttgtcttttgccttttttctagccactgcctcagttttctgctgtgagccaggttcaaagtgtgggtttagctttatcccgtagccctgttttattgcaccacagcatatcagggctgaagggacttgacttttatcttttgattttaggggtaagctggggaggctgggccaggtggtagttttataggccagtgacccgccaggccttgggccctgtgagtcctggtcttgagtccttcctgtatccacacattgcagtcagtggcggggctagtctctgtgaccaagtagtgtccatgtctccgtctgacagtctcagctccagcccgtttgataaagaagtgttggctatctgcctatttccttttcatagtggcacacagattatccctagccagctgtggtggtttagaattgttttagatgccagcttcccccagcaggtctgtgaaggactattactgattgttttgtttgtttgttttttattgtaatgacagcctgggaaggaatttttcccattggatgagtcgggcaatgtggttcccagtgacaccaacattctggacacgtgggcggtaagacagcccctggttggaccttgttttcgtgactggttccctcttcttcttagaagcaatctgcctgccatttctcacattgcattttgttattctgtccctgttgtttgctctctctttttttttttttgagttggggtctccctctgttgcccaggctggagtgcagtggcacaatctcggctcactgcaagctccacctcctgggttcacgccattctcctgcctcagcctcctgagtagctgggactacaggtgcccgccaccacgcccggctaatttttttgtatttttagtagagacggggtttcaccgtgttagccaggatggtcttgatctcctgacctcgtgatccgcccgcctcagcctcccaaagtgctgggattacaggcgtgagccaccgcgcccggcctgtttgctcttcttatctcattaaagccagcctggctttcgtctcactgtctcttagcttctgaaaggtctaattcactggcactgaagagtgagggcaggacccgttaaactggtgcaaacgacacttctgatcctgtcctttctgggttcgtttctgcaccacacagacacttttctctgtaggccatggaagagctggtggatgaagggctggtgaaagctattggcatctccaacttcaaccatctccaggtggagatgatcttaaacaaacctggcttgaagtataagcctgcagttaaccaggtaaacatcccccaacgacactggcattgcaacgttgctgttttactgtctgatgctggtagggatttctcgtccacctcacccagcaagtggccaggaggcagctcctgggtgttggcagagtcctgagttatgattcagcatgacacagcacatctgggagaggaaacttacaggaaagaaagagtttgagcctggcttgtgacgatttggggtctgacaaagaggcagccatgagatcctcagaactgtgatagttgataagtggctaaagccaagaaaagcctctagggcccatgagaacatctataataagataataataacaccaaggatactacctgggaactgtaggaagttacagcttatctgggaaatagggtgtttgttatacatagttttctgtatggggtagggagggactttagcataggaagcttcatctgtgtcccatcagcgtgtagccacagtgctcccatcagctcttgtttttgcatttgcagattgagtgccacccatatctcactcaggagaagttaatccagtactgccagtccaaaggcatcgtggtgaccgcctacagccccctcggctctcctgacaggccctggtgagcttcccacaggctcatgctcctgtgtcacttggtgaagattagaaaagactaaaaagaacaattcagcctcaaggaagatgtttctgggggtgatctgcaaaacttcccctgagcctcttgctggttttcctggaagggttggagtttctagtagcaagacacctactctttttccaaagggttgggggttcttaacccagctctgaggaggagactggtggctgccctctgaggtcgatgggaggattagggttggggtttctggctgctgtgagtggtggccatgatggtgtgacctggactgtctttctatcctcagggccaagcccgaggacccttctctcctggaggatcccaggatcaaggcgatcgcagccaagcacaataaaactacagcccaggtacagccacttcaggtgttgctgaccgtccacaactgcctgcattcctgacagtcctgttagccaagaggaggaagtgactgagcctgttacaccctcacaggaagtatggttaggggtcctcaagtacagagtggaaagggcacagatcggggttttagaagactctggcatgggctcttagattaatagtgcctgcccccactactgcaagggtgactgccacgagggccagcgcttgttcattcatgtggaacctcatctgtacaaatgtaagagctcttagccgtgcagggaatgttctttctcctgagtggtagtgtgcatttctagccagtggagggcctcatgtggtctcatgatatgcctgagacactgaagcgtgtggcacagtggctagcgcaggactctggagtcagatctggacctgaatgcgtcgcctacctgttgctagctgtgacctgacatcttggagcccctctctgatcacctgtggagttctagcacgtccttctgcaggttgtgtgtgtgagagactgagatgatgggtgcgagtgcctggcatgtatacacactcactgtctccttgggctcacaggtcctgatccggttccccatgcagaggaacttggtggtgatccccaagtctgtgacaccagaacgcattgctgagaactttaaggtaagatcttggctggtcaggcctggccctcctccatggagtgggggatgggggaggcctctcatcctgtctctggagtgtcatctgtgggatcaccaccatcctctcttctgaggccagggagctgtggcgagcaagccaagactgagactgacacctcaccagtggagccgtgtgcaggggcaggccttgggcctccagggccccgtgctgtgcacgcatacacctacacctttgctcaggcccttcagccacaccgagaggttacccggggagaatctcgctcttgagcttcactgcctggacctgccctgcactggagctctgtcagctaccagctgcataaccctgggcaagtcgcttaactgctcgatgcctcagcttccctatctataaaataatatccattttgagggttgttacgaagattaagtgatgcatcagagcacttcacatggtgcttaataccttagtaagtgatcgagaaatgttgctgtgctgataagtccagggactgcaaaggaccctctgggccatttagtccattcaccagctctgcaaggagttcgtgtgatgatgggcagagcctagacctgtgtcaggactctggcgaagcgctcagtgtggcagctggagcagctcaggctacttggcgccacctccctccctgcctgcccttgcagcattggtggcgggggtgtgggggagggggggggcacgcccaaaggagagcagctcaggggagtcctgatggcccaagagcctctgttctcactgagcaaagtggctctgccagggatgctctgtctttatccaagatctgtgccgagcgcagcacagccatgggcctggggctcttgggcaggccccagcttaggtgagcagctgattttgcccaattccactgcccaaagactgtggccatagtgcctgccacctgtcacatatgagacagggatgagagtgatccctgaagagggaagtggagctcattgtttaggagaggagacgtttttcttcttacgtgcaggtagatgttctaagtacacaagataaaatggcaaaacagtggccactgagggaaaggcatatcccaccagagatgcatcgtttgtttcgtatttcattttttaaggaattaggaaagaaaaaataaatgactaatctcaaaatgctaacctaattattttatttttgcatcttactgtatagcttttgcacatttttctgtgatcataaccacagagcctgtagcagactctctttctgcacattctgtgatcatctgaagctaagagcccttcggctctgggagtgtgtgtcagcctcttgtgggtctcacccagtggtgcctgcctgcctccctccctcccagcctccatgccagctgagttctgtaccgtctgctgccttcatgcaggtggttctcttctcccagtatacctttcttgccctctctgtcaaaatcttatacttcttaggccatgtcaaataccatctcctcttcggctgtggttgttcagtttcccggctggaatccctttggaatacctctgcccttcctgtgctgttgagtgtcttatacagtgtgtgattagttatttgtattacagttgtagaaatacctctgccctttgtttgcttcataaaatagaggcaaagacacatcttacccataattatattcctcactgcaacttatcacaatagaagtgctcagtagtgttttttgaaatgaaataaaatgtatcatttccctgggaggttctggaaaggaaggactgtttccatcgttccatatattatctagcccaacatgcacatttcctgaatactaatatatcagcttttcctaagttcagtttatcttatcgctcctctctgctcaaagccatggaagacatctgggtcctgcacagtattttgtcttttatttttatttgcagagcttactggatttttttgtttgttttttaggtctttgactttgaactgagcagccaggatatgaccaccttactcagctacaacaggaactggagggtctgtgccttgttgaggtgagtgagccccagtgtgaggagcagttcccaggagatattcagatgctgcacagcaagagagtgatccctgcaggcctcagcgccaatgtgctctcgtcacagctgttcttgctttgcacattaccttgctttctcagccacgtgacttgcatcagctgaactgcatggagccaagatgccaagagatccgctcttgatatcttcttccagccctggggcattgttggcctggcctgaagtaactacatattttggctttccattcaatttttaaatcctagcagttcatctgaaactaggtgatcccaggtgaccctggatcaaactctggaagaatttgcctgtgactttttgtcttaatgaccctaatgaaaggggtttggtcgggagcccctggggctgaggtttgagacagtcccggctctgacctggtaagccacatggctccaaaacaggccctccctctgagcagcaggcacctcagttcctccagccacacatcctctggttctgtttcctctgcatggtagtttggaaggagctgcctaggaaggttttctgtggggttttctcttttcctaccattacaataactaggtgacgcatgtaactcactttctgctttcaagaaggaacttctcattttgcagtggagactgagactctgtttctctctcactacccttattcttgtctgagctctgtgtttccacactctctcctttagtacttcctcttctaagcaaaatgtgaacattccctaagatggggtgggctaccagctttccctttccagcgtctgtcccatctccctccccggagtttgcactgctggtggcttcagccaccgccccaggcagatgtttgcccagtctgcgtcctggacaataactctggccctgcttcaggcatatgcgtgcctggcaagcctgctgctgccacactgtggcctcacactcaaaataggccgcaaacagagcatgtcacaggcagccccaggccagtgccctttcagacatctctgttaatgctacttagccctagacattaaaatcatttttggatctagtggacaaggaggagaggagtgaatcagtcaccaagctacattcgttctcctttcagaatctctcccattcatccgtcttccttttcccctcccacttgagacagagtggattgaagctttcagggctgaggaaacacagtggttttctcattgctccctttacagttaggctcctctgccctccagtattgtgctagaaacttctgttcgattatcctgttgcgtggcggtgagtgggaggaagaggctagccctgacatgctcaggtctcctagggagttttcgctaactacacacacaccacccctctggcagataatgctttactgagcctgacctgctgatgtcagtgtcactgtgggtatacaaaaaaggcagaccatggagaacttttgaataagagttacagactgtaagcacccagctcagattacataactctggccttgcgtgagctgcccagctatctgtccagtgttcctgcctttatgttcctagcacagccattggccctactagatggacagtgagtgagccttgacaaacacgtctgcacctgtgctccctgccatgtgctccctgccctgtgctccctgccctgtttccaatataaaatcgccgaccgttgattgggctcacttcagcccccactgatacaaggaggctccctctgtcctcagagcacttgccgttgcttctcttatcctttgagattcagtcacatacagacttttaacatccctttttttttttgaaatgggctcctactctgtcacccaggctggaatgtaatggcatgatcacagctcactgcagccttgacctcctgggctcaaatgatcctcccacctcagcctcccaagtagctggggccatgggcatgcaccaccatgcctggttaatttaaaaagtttttttattaagatggaaatcttgctatgttgcccaggctagtcgcaaacccctagcctcaagtgatcctctcgcttcagcctgggattacaggcgtgagccactgtgtctggcctgccttctcatattattacacaagatgtttaagaccacaaaatactgtctggtcctgccagctagactgagttactggaaggcagaaatcgtgttgccgtcctgcacggcccaggcacagtgctgggcacatggggagggtggtgtccctcacacatgtgctgcttgatgacagtagtggtagaggggtctggagggtgagttgcttcccccagctgcagaaccatcatctgcacactcagaactagcttgttgcctctgaggttctattttatttcttcagtgagacacatcatttctctgttgggagttgggtatagtcaaaatcacctcttcaaaaagcaactgttttctcctttctcctgacagctgtacctcccacaaggattaccccttccatgaagagttttgaagctgtggttgcctgctcgtccccaagtgacctatacctgtgtttcttgcctcatttttttccttgcaaatgtagtatggcctgtgtcactcagcagtgggacagcaacctgtagagtggccagcgagggcgtgtctagcttgatgttggatctcaagagccctgtcagtagagtagaagtctcttccagtttgctttgcccttctttctaccctgctggggaaagtacaacctgaatacccttttctgaccaaagagaagcaaaatctaccaggtcaaaatagtgccactaacggttgagttttgactgcttggaactggaatcctttcagcaagacttctctttgcctcaaataaaaagtgcttttgtgagcttgg

(19) United States
(12) Patent Application Publication
(10) Pub. No.: US 2024/0374693 A1
(43) Pub. Date: Nov. 14, 2024

(54) TREATMENT AND DETECTION OF INHERITED NEUROPATHIES AND ASSOCIATED DISORDERS

(71) Applicants: University of Miami, Miami, FL (US); University of Rochester, Rochester, NY (US); UCL Business LTD, London (GB)

(72) Inventors: Stephan L. ZUCHNER, Miami, FL (US); Adriana REBELO, Miami, FL (US); Andrea CORTESE, London (GB); Rong Grace ZHAI, Miami, FL (US); David N. HERRMANN, Rochester, NY (US)

(21) Appl. No.: 18/788,923

(22) Filed: Jul. 30, 2024

Related U.S. Application Data
(63) Continuation of application No. 17/719,580, filed on Apr. 13, 2022, now Pat. No. 12,083,168.
Application No. 17/719,580 is a continuation of application No. 17/517,227, filed on Nov. 2, 2021, now abandoned.

(51) Int. Cl. A61K 38/44; A61K 31/198; A61K 31/352; A61K 31/4184; A61K 31/4188; A61K 31/426; A61K 31/428; A61K 31/473; A61K 31/499; A61K 31/502; A61K 31/517; A61K 31/7105; A61K 38/46; A61K 48/00; A61P 25/28; C12N 9/04; C12N 15/113; C12N 15/86; C12Q 1/6883; G01N 33/66

(52) U.S. Cl. CPC A61K 38/443; A61K 31/198; A61K 31/352; A61K 31/4184; A61K 31/4188; A61K 31/426; A61K 31/428; A61K 31/473; A61K 31/499; A61K 31/502; A61K 31/517; A61K 31/7105; A61K 38/465; A61K 48/005; A61P 25/28; C12N 9/0006; C12N 15/1137; C12N 15/86; C12Q 1/6883; C12Y 101/01014; G01N 33/66; C12N 2310/11; C12N 2750/14143; C12Q 2600/156

(57) ABSTRACT

The present disclosure relates to methods of detecting and treating inherited neuropathy.

### CROSS REFERENCE TO RELATED APPLICATIONS AND INCORPORATION BY REFERENCE OF MATERIAL SUBMITTED ELECTRONICALLY

This application is a continuation of U.S. patent application Ser. No. 17/719,580, filed Apr. 13, 2022, which is continuation of U.S. patent application Ser. No. 17/517,227, filed Nov. 2, 2021, which is a continuation of International Patent Application No. PCT/US2020/031708, filed May 6, 2020, which claims the benefit of U.S. Provisional Patent Application No. 62/844,370, filed May 7, 2019, and U.S. Provisional Patent Application No. 62/987,151, filed Mar. 9, 2020, each of the foregoing are incorporated herein by reference in their entireties.

### GRANT FUNDING DISCLOSURE

This invention was made with government support under grant numbers NS065712, NS075764 and GM119018 awarded by the National Institutes of Health (NIH). The government has certain rights in the invention.

### SEQUENCE LISTING

This application contains a Sequence Listing which has been submitted electronically in XML format. The Sequence Listing XML is incorporated herein by reference. Said XML file, created on Apr. 12, 2024 is named 761350_000424_SL.xml and is 525,555 bytes in size.

### FIELD OF THE INVENTION

The present disclosure relates to methods of detecting and treating inherited neuropathy. BACKGROUND

Peripheral neuropathies are amongst the most frequent neurodegenerative diseases, with diabetic neuropathy and hereditary origins amongst the most common mechanisms of action. For the inherited neuropathies, also known as Charcot-Marie-Tooth disease (CMT), the remaining diagnostic gap of patients is ˜50%. In our understanding, CMT represents an umbrella concept for clinically and genetically heterogeneous inherited monogenic highly phenotypically penetrant conditions affecting the peripheral nerves. CMT is classified depending on conduction velocity as demyelinating (CMT1) and axonal (CMT2) types. Distal hereditary motor neuropathy (dHMN) represents a form of CMT2 in which the burden of disease falls predominantly or exclusively on motor nerves (Rossor, Tomaselli, and Reilly 2016). A similar condition includes ALS4 (juvenile dHMN+brisk reflexes as sign of upper motoneuron involvement). As opposed to CMT1, for which over 90% of cases have mutations in known genes, only 20 to 30% of CMT2 and distal HMN patients receive a genetic diagnosis (Fridman et al. 2015).

### SUMMARY

The disclosure provides a method of treating and/or detecting inherited neuropathy. In various aspects, the method comprises detecting the presence of a mutation in the sorbitol dehydrogenase (SORD)) gene in a sample from a subject. In various embodiments, the SORD mutation is a DNA variant classified as pathogenic or likely pathogenic according to American College of Medical Genetics and Genomics (ACMG) criteria. Optionally, the method comprises diagnosing the subject with inherited neuropathy when the presence of a mutation in the SORD gene is detected. Optionally, the method comprises administering to the subject a composition that comprises an agent selected from the group consisting of an aldose reductase inhibitor; an aldose reductase antisense oligonucleotide; a polynucleotide that encodes a SORD peptide; a SORD peptide; an agent that blocks expression of a mutant SORD gene; and an agent that corrects the mutation in SORD gene. In various aspects, the method comprises administering to the subject Alrestatin, Epalrestat, Diepalrestat, Fidarestat, Imirestat, Lidorestat, Minalrestat, Ponalrestat, Ranirestat, Salfredin B₁₁. Sorbinil, Tolrestat, Zenarestat, or Zopolrestat (or a combination thereof). In various aspects, the method comprises administering to the subject an aldose reductase antisense oligonucleotide; a polynucleotide that encodes a SORD peptide; an agent that blocks expression of a mutant SORD gene; an agent that corrects the mutation in SORD gene; or a combination of any of the foregoing. In various aspects, the method comprises administering to the subject a SORD peptide. Administration of a combination of any of the foregoing is also contemplated. Optionally, the method comprises measuring sorbitol levels in a sample from the subject.

Also provided is use of an (i) aldose reductase inhibitor (e.g., Alrestatin, Epalrestat, Diepalrestat, Fidarestat, Imirestat, Lidorestat, Minalrestat, Ponalrestat, Ranirestat, Salfredin B₁₁, Sorbinil, Tolrestat, Zenarestat, and/or Zopolrestat); (ii) an aldose reductase antisense oligonucleotide, a polynucleotide that encodes a SORD peptide, an agent that blocks expression of a mutant SORD gene, and/or an agent that corrects the mutation in a SORD gene; and/or (iii) a SORD peptide for the treatment of inherited neuropathy (or use in the preparation of a medicament for treatment of inherited neuropathy) in a subject which has been tested for the presence of a mutation in the sorbitol dehydrogenase (SORD) gene.

The disclosure further provides a method of characterizing a neuropathy in a mammalian subject, the method comprising measuring the level of sorbitol in a subject suffering from a neuropathy, wherein a sorbitol level of greater than about 10 g/L indicates that the neuropathy is associated with a mutation in the sorbitol dehydrogenase (SORD) gene. The disclosure also provides a method of evaluating the efficacy of a treatment for an inherited neuropathy in a subject, the method comprising administering to the subject an agent selected from the group consisting of an aldose reductase inhibitor (e.g., Alrestatin, Epalrestat, Diepalrestat, Fidarestat, Imirestat, Lidorestat, Minalrestat, Ponalrestat, Ranirestat, Salfredin B₁₁, Sorbinil, Tolrestat, Zenarestat, and/or Zopolrestat), an aldose reductase antisense oligonucleotide, a polynucleotide that encodes a SORD peptide, a SORD peptide, an agent that blocks expression of a mutant SORD gene, and an agent that corrects the mutation in SORD gene (or a combination of any of the foregoing); and measuring the level of sorbitol in a subject.

It is understood that each feature or embodiment, or combination, described herein is a non-limiting, illustrative example of any of the aspects of the disclosure and, as such, is meant to be combinable with any other feature or embodiment, or combination, described herein. For example, where features are described with language such as “one embodiment,” “some embodiments,” “various embodiments,” “related embodiments,” each of these types of embodiments is a non-limiting example of a feature that is intended to be combined with any other feature, or combination of features, described herein without having to list every possible combination. Such features or combinations of features apply to any of the aspects of the invention.

The headings herein are for the convenience of the reader and not intended to be limiting. Additional aspects, embodiments, and variations of the invention will be apparent from the Detailed Description and/or drawings and/or claims.

### BRIEF DESCRIPTION OF THE DRAWINGS

FIGS. 1A-F. SORD gene and pedigrees. Biallelic mutation in SORD) cause autosomal recessive dHMN/CMT2. (FIG. 1A) Representative pedigrees of dHMN/CMT2 families carrying biallelic mutations in SORD. The squares indicate males and the circles females. The diagonal lines are used for deceased individuals. Patients are indicated with filled shapes. (FIG. 1B) Schematic diagram showing all exons, introns and untranslated regions (UTRs) of SORD on the basis of NCBI Reference Sequence: NM_003104.6. The gray and white boxes represent the coding sequence and UTRs of SORD, respectively. Variants identified in the families considered in the present study map throughout the coding region of the gene. The nonsense c.757delG; p.(Ala253GlnfsTer27) variant on exon 7, was identified at particular high frequency. (FIG. 1C) Distribution of mutation across SORD protein domains. (FIG. 1D) SORD protein orthologs alignments showing that the four missense substitutions identified in dHMN/CMT2 families in this study are located at highly conserved residues across species from humans to elephants (FIGS. 1E and 1F) Magnification the nucleotide sequence of a highly homologous region in exon 7 in SORD (reverse strand) and SORD2P (forward strand). Nucleotides differing in SORD2P from SORD are indicated with an arrow, including a FIG. 1C deletion in SORD2P. Representative electropherograms shows that in SORD the c.757delG; p. (Ala253GlnfsTer27) variant found in homozygous state in dHMN/CMT2 patients and heterozygous state in available patents (right box, upper plot) is absent in biallelic state from healthy controls (right box, lower plot), but it is fixated in SORP2P (left box, lower plot).

FIGS. 2A-C. Decreased SORD expression and sorbitol accumulation in patients fibroblasts. (FIG. 2A) Schematic representation of the two-step polyol pathway converting glucose to fructose. (FIG. 2B) Immunoblot showing protein level of SORD using the polyclonal antibody ab189248 and normalized to Tubulin in healthy control (n=4, lane 1-4), heterozygous carriers of c.757delG; p.(Ala253GlnfsTer27) variant in SORD (n=2, lane 10-11) and patients carrying homozygous c.757delG; p.(Ala253GlnfsTer27) change (n=4, lane 5-8) or compound heterozygous c.757delG; p.(Ala253GlnfsTer27) variant together with a second nonsense c.895C>T; p.(Arg299Ter) mutation (n=1, lane 9). (FIG. 2C) Levels of intracellular sorbitol as measured by UPLC and normalised to protein content in healthy controls (n=5) and patients carrying biallelic nonsense mutations in SORD (n=5). The graphs show the mean ±s.d. and data distribution (dots). A two-tailed t-test was performed to compare SORD encoded protein (FIG. 2B) or sorbitol level (FIG. 2C) across groups. Statistical significance is indicated as *, ** or *** if P-value<0.05, <0.01 or <0.001, respectively. All experiments were repeated independently twice with similar results.

FIGS. 3A-F. Loss of Drosophila Sord2 causes age-dependent synaptic degeneration. (FIG. 3A) 3D structure of Drosophila visual system showing the lamina, medulla, and lobula. The xy- and xz-planes showing the photoreceptor terminals and lamina neurons are indicated. (FIG. 3B) Lamina of yw control fly at 2 DAE. The organized lamina cartridges and columnar photoreceptor neurons are shown in the xy-plane and xz-plane, respectively. (FIG. 3C) Laminae of Sodh2^{MB01265/MB01265} homozygous flies at 2 DAE and 10 DAE. Arrowheads indicate the lamina vacuoles. Boxes indicate higher magnification areas of the lamina. The intensity of BRP is indicated. Dotted lines indicate the area of lamina vacuoles. Scale bar: 30 μm. (FIG. 3D) Quantification of the vacuole number, size, and BRP intensity. A total of 3 laminae of each group were quantified. Data are presented as mean ±s.d. Statistical analysis was performed using Two-Way ANOVA followed by post-hoc Tukey's multiple comparison test. *P<0.05, **P<0.01, ****P<0.0001. (FIGS. 3E-3F) Locomotor activity of control flies (yw) and Sodh2^{MB01265/MB01265} (FIG. 3E) or Sodh1 and Sodh2 pan-neuronal double knockdown (RNAi) (FIG. 3F) flies. n=10 in each group. Data are presented as mean ±s.d. Statistical analysis was performed using Two-Way ANOVA followed by post-hoc Tukey's multiple comparison test. ****P<0.0001.

FIGS. 4A-G. Treatment with aldose reductase inhibitors Epalrestat and Ranirestat decrease sorbitol level and restore function. (FIG. 4A) Intracellular sorbitol level as measured by UPLC and normalised to protein content in fibroblasts from healthy controls (n=5, circle dots) and patients carrying biallelic nonsense mutations in SORD (n=5, square dots) after three days of treatment with Epalrestat 100 μM, Ranirestat 10 μM or DMSO. (FIG. 4B) Sorbitol level as measured by UPLC from brain/head homogenates and normalised to protein concentration from wild-type (yw, empty circle dots), Sodh2^{MB01265/MB01265} (full circle dots) and neuron-specific knock-down of Sodh1 and Sodh1 by RNAi (square dots) Drosophilae at 10 days after eggs enclosure. Sodh2 Mimic and Sodh1 and Soh2 RNAi Drosophilae were fed with either 80 μM Epalrestat, 80 μM Ranirestat or DMSO. The graphs show the mean ±s.d. A two-tailed t-test was performed to compare sorbitol level. Statistical significance is indicated as *, ** or *** if P-value<0.05, <0.01 or <0.001, respectively, unless otherwise specified. All experiments were repeated independently twice with similar results. (FIG. 4C) Locomotor activity of control flies (yw) feeding with DMSO, Sodh2^{MB01265/MB01265} flies feeding with DMSO, 80 μM Epalrestat, or 80 μM Ranirestat (n=10 in each group). Data are presented as mean ±s.d. Statistical analysis was performed using Two-Way ANOVA followed by post-hoc Tukey's multiple comparison test. *P<0.05, ***P<0.001 (FIGS. 4D-4F) Laminae of Sodh2^{MB01265/MB01265} homozygous flies at 10 DAE and 40 DAE fed with DMSO (FIG. 4D), 80 μM Epalrestat (FIG. 4E), or 80 μM Ranirestat (FIG. 4F). Arrowheads indicate the lamina vacuoles. Boxes indicate higher magnification areas of the lamina. The intensity of BRP is indicated. Dotted lines indicate the area of lamina vacuoles. Scale bar: 30 μm. (FIG. 4G) Quantification of the vacuole number, size, and BRP intensity of (FIGS. 4D-4F). n=3. Data are presented as mean ±s.d. Statistical analysis was performed using Two-Way ANOVA followed by post-hoc Tukey's multiple comparison test. *P<0.05, **P<0.01, ****P<0.0001.

FIG. 5. Pedigrees of families carrying biallelic mutations in SORD. The squares indicate males and the circles females. The diagonal lines are used for deceased individuals. Patients are indicated with filled shapes.

FIGS. 6A-B. Double knockdown of Drosophila Sodh1 and Sodh2 lead to age-dependent synaptic degeneration. (FIG. 6A) Laminae of Sodh1 and Sodh2 double knockdown homozygous flies at 2 DAE and 10 DAE. Arrowheads indicate the lamina vacuoles. Boxes indicate higher magnification areas of the lamina. The intensity of BRP is indicated. Dotted lines indicate the area of lamina vacuoles. Scale bar: 30 μm. (FIG. 6B) Quantification of the vacuole number, size, and BRP intensity. A total of 3 laminae of each group were quantified. Data are presented as mean ±s.d. Statistical analysis was performed using Two-Way ANOVA followed by post-hoc Tukey's multiple comparison test. *P<0.05, **P<0.01, ****P<0.0001.

FIG. 7. Treatment with aldose reductase inhibitors Epalrestat and Ranirestat restore locomotor function in Sodh1 and Sodh2 double knockdown flies. Locomotor activity of control flies (yw) feeding with DMSO (dots, first data point from the left for each DAE point indicated), or flies with neuronal specific knockdown of Sodh1 and Sodh2 feeding with DMSO (squares, second data point from the left for each DAE point indicated), 80 μM Epalrestat (squares, third data point from the left for each DAE point indicated), or 80 μM Ranirestat (squares, forth data point from the left for each DAE point indicated). n=10 in each group. Data are presented as mean ±s.d. Statistical analysis was performed using Two-Way ANOVA followed by post-hoc Tukey's multiple comparison test. ***P<0.001, ****P<0.0001.

FIG. 8. An illustration of an exemplary expression vector encoding the SORD peptide (pAAV-SORD).

FIG. 9. An exemplary complete AAV vector DNA sequence including the SORD coding sequence (pAAV-SORD) (SEQ ID NO: 1).

FIG. 10. SORD primer sequences and thermocycling conditions. PCR: polymerase chain reaction; Fw: forward; Rv: reverse.

FIG. 11. Clinical features of patients with hereditary neuropathy and carrying biallelic mutations in SORD.

FIG. 12. Clinical features of patients affected by hereditary neuropathy and carrying the biallelic mutations in SORD. Categorical data are expressed as N (%) if data is available in all individuals or N/number individuals considered (%). Continuous variables are expressed as mean±standard deviation (min-max). CMT, Charcot-Marie-Tooth, dHMN, distal hereditary motor neuropathy.

FIG. 13. Fasting sorbitol level in serum from ten unrelated healthy controls and ten patients carrying biallelic p.Ala253GInfsTer27 mutations in SORD. The graphs show the mean ±s.d. and data distribution (dots), and the p-value of two-tailed t-tests comparing SORD protein and sorbitol levels across groups-*P<0.05, **P<0.01, and ***P<0.001. All experiments were twice repeated independently.

FIGS. 14A-14C. Exemplary vector design for SORD gene replacement therapy. (FIG. 14A) AAV-9 packaged vector design for a SORD gene replacement therapy. CB7 promotors have been shown to be effective in driving high expression, followed by the SORD cDNA (NCBI Reference Sequence: NM_003104.6), a Posttranscriptional Regulatory Element (WPRE) to further enhance expression and target specificity, and the transcription termination poly (A) element. Further origin of replication (pUC-ori) and ITR sequences (inverted terminal repeat). (FIG. 14B) SORD cDNA sequence. (FIG. 14C) SORD polypeptide sequence.

FIGS. 15A-15D. Significant knock-down of aldose reductase (AR) (AKR1B1 gene) via an antisense oligonucleotide (ASO) (AR 1A, (SEQ ID NO: 22)). Targeting ASO (AR 1A) sequence and ASO-S scrambled sequence (AR-S 1A, (SEQ ID NO: 47)) are shown in FIG. 15A. FIG. 15B shows the modifications to the nucleotide backbone of the ASOs. This was carried out in a SORD patient fibroblast and control fibroblasts and normalized to μ-tubulin and measured via Western blot (FIGS. 15C-15D). A further control is a scrambled version of the ASO-S (AR-S 1A) exhibiting random nucleotides was used (FIG. 15C).

FIG. 16. A table of antisense oligonucleotide sequences and target sites in Homo sapiens aldo-keto reductase family 1 member B (AKR1B1), exon targets only. Filter criteria: A) 40%<=GC %<=60%; B) Antisense oligo binding energy<=−8 kcal/mol; C) No GGGG in the target sequence.

FIG. 17. A table of antisense oligonucleotide (ASO) sequences an target sites in Homo sapiens aldo-keto reductase family 1 member B (AKR1B1), exon targets only. Filter criteria: A) 40%<=GC %<=60%; B) No GGGG in the target sequence; C) Average unpaired probability for target site nucleotides>=0.5; D) For each peak in the accessibility profile that is above the threshold probability of 0.5, all sites targeted to this same peak are ranked by their average unpaired probability (the higher the better) and at most n sites are selected for each peak, where n is determined by max([width of peak/site length], 2); E) Among sites satisfying criteria A-D, the top 20 unique ones with the highest average unpaired probability are listed.

FIG. 18. A table of antisense oligonucleotide (ASO) sequences and target sites in Homo sapiens aldo-keto reductase family 1 member B (AKR1B1), hg19_dna range=chr7:134127102-134143944 (intronic targets only). Filter criteria: A) 40%<=GC %<=60%; B) No GGGG in the target sequence; C) Average unpaired probability for target site nucleotides>=0.5; D) For each peak in the accessibility profile that is above the threshold probability of 0.5, all sites targeted to this same peak are ranked by their average unpaired probability (the higher the better) and at most n sites are selected for each peak, where n is determined by max([width of peak/site length], 2); E) Among sites satisfying criteria A-D, the top 20 unique ones with the highest average unpaired probability are listed.

### DETAILED DESCRIPTION

The disclosure provides a method of detecting and/or treating inherited neuropathy and related inherited conditions.

Inherited (or hereditary) neuropathies include, but are not limited to Charcot-Marie-Tooth disease (CMT), hereditary motor and sensory neuropathy, hereditary motor neuropathy, distal hereditary motor neuropathy (dHMN), axonal neuropathies, intermediate neuropathies, and amyotrophic lateral sclerosis type ALS4.

In various aspects, the disclosure provides a method wherein the presence of a mutation in the sorbitol dehydrogenase (SORD)) gene is detected in a sample from a subject. The mutation may be detected by examining the DNA sequence of the gene, examining RNA, or examining proteins with mutations that result in some loss of function.

Disclosed herein is the identification of biallelic mutations in the Sorbitol dehydrogenase gene (SORD)) associated with the most frequent recessive form of CMT. SORD encodes sorbitol dehydrogenase, an enzyme which converts sorbitol to fructose. It belongs to the two-step polyol pathway previously identified as pivotal to nerve damage in hyperglycemic condition of diabetes. Forty-two cases of CMT across different ethnicities were identified as carrying a nonsense mutation in SORD, c.757delG; p.Ala253GlnfsTer27, either in homozygous or compound heterozygous state. By screening the p.Ala253GlnfsTer27 change in additional cases and multiple control sets, this variant was established as one of the most common pathogenic alleles in men inherited according to Mendel's law (MAF=0.003). Patient fibroblast cultures exhibit a complete loss of SORD protein as well as loss of intracellular sorbitol accumulation, which causes tissue damage. Loss of Sodh1 in Drosophila led to synaptic degeneration and progressive motor impairment. Notably, reduction of polyol influx by treatment with aldose reductase inhibitors fully rescued intracellular sorbitol levels in patient fibroblasts and a Sodh1 Drosophila model. In the latter model, the treatment also completely ameliorated motor and eye phenotypes. Together, these findings demonstrate a major role of the polyol pathway and sorbitol accumulation in hereditary neuropathies and establish the molecular cause for a potentially treatable condition in a significant fraction of cases. These findings also represent an example of converging pathomechanisms of hereditary and acquired neuropathies with a broader impact in the field of diabetes.

Thus, in various aspects of the disclosure, the method comprises detecting the SORD gene mutation 753delG; p.(Ala253GlnfsTer27), c.757delG; p.Ala253GlnfsTer27, c.28C>T; p.Leu10Phe, c.316_425+165del; p.Cys106Ter, c.329G>C; p.Arg110Pro, c.298C>T; p.Arg100Ter, c.295C>T; p.Arg299Ter, c.964G>A; p. Val322Ile, c.458C>A; p.Ala153Asp; a deletion of individual or multiple coding exons or the entire SORD gene via a copy number variation; or any protein truncating mutation and/or mutation that leads to a “loss of function” or a hypomorphic function of the protein.

In various aspects, the SORD mutation is detected using DNA sequencing methods such as whole exome sequencing, whole genome sequencing (WGS) and/or next-generation sequencing (NGS), allele specific oligonucleotides, polymerase chain reaction (PCR), quantitative or real-time PCR (qPCR), multiplex PCR, nested PCR, Amplification Refractory Mutation System (ARMS) PCR, Multiplex ligation-dependent probe amplification (MLPA), Denaturing gradient gel electrophoresis (DGGE), Single-Strand Conformation Polymorphism (SSCP), Protein Truncation Test (PTT), RFLP, DNA microarray, RNA-seq, using CRISPR-based mutation detection (e.g., CRISPR-Chip, Hajian et al., Nature Biomedical Engineering 3, 427-437 (2019)) or other DNA or RNA mutation detection methods suitable for mutation detection.

In various aspects, the SORD mutation is detected by examining proteins using western blotting (immunoblot), High-performance liquid chromatography (HPLC), Liquid chromatography-mass spectrometry (LC/MS), antibody dependent methods such as enzyme-linked immunosorbent assay (ELISA), uwtyjns rr r zstuwjhnunyfynts1uwtyjns rr r zstxyfnsnsl1protein chip methods or other protein detection methods suitable for mutation detection.

Optionally, the method further comprises measuring sorbitol levels in a sample of the subject. Methods of measuring sorbitol include, e.g., enzymatic assays, fluorimetric assays, chromatography-based methods, and spectroscopy-based methods. An exemplary method of sorbitol measurement is provided in the Examples.

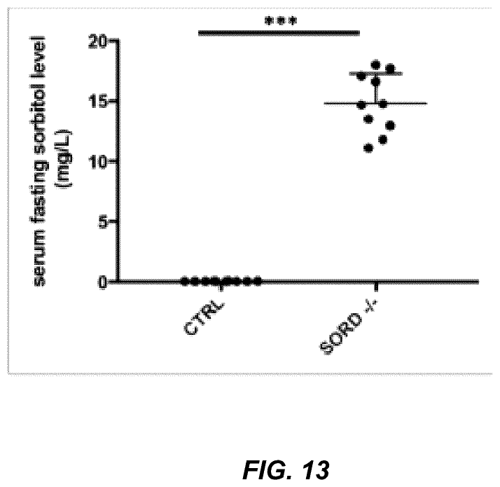

The disclosure further provides a method of characterizing a neuropathy (e.g., inherited neuropathy) and related conditions involving a SORD mutation. In various aspects, the method comprises measuring sorbitol levels in a biological sample of a subject suffering from a neuropathy. In various aspects, the method comprises detecting increased levels of sorbitol in the biological sample. By “increased levels of sorbitol” is meant, e.g., sorbitol levels above about 10 mg/L. SORD-related neuropathy leads to high levels of sorbitol in patients, as described in the Examples and FIG. 13. As such, detection of sorbitol levels above about 10 mg/L indicates that the neuropathy is an inherited neuropathy associated with a SORD mutation, thereby allowing a clinician to characterize the neuropathy afflicting the subject. Optionally, the method comprises a treatment step comprising administering to the subject an agent selected from the group consisting of an aldose reductase inhibitor; an aldose reductase antisense oligonucleotide; a polynucleotide that encodes a SORD peptide; a SORD peptide; an agent that blocks expression of a mutant SORD gene; and an agent that corrects the mutation in SORD gene.

In various aspects, the disclosure provides a method comprising identifying a mutation in the sorbitol dehydrogenase (SORD)) gene in a sample from a subject before or after a step of measuring sorbitol levels in the subject. In this regard, the method may be used to confirm a diagnosis of inherited neuropathy. Similarly, the disclosure provides a method for identifying a SORD mutation that is pathogenic, the method comprising measuring sorbitol levels in a subject comprising a mutation in the SORD gene. The presence of increased sorbitol levels (e.g., greater than about 10 mg/L) indicates that the SORD mutation is pathogenic.

Alternatively (or in addition), the method may be used to evaluate the efficacy of a treatment for an inherited neuropathy in a subject. In this regard, the method comprises administering a therapy to the subject, then measuring sorbitol levels in a biological sample. A decrease in sorbitol levels compared to the level of sorbitol observed pre-treatment (e.g., a reduction of sorbitol levels below about 10 g/L) indicates an improvement in the subject's condition. The materials and methods described herein may also characterize patient compliance in taking medication for treatment of SORD-related inherited neuropathies or monitor the success of candidate therapeutics in clinical trials.

The sample may be any biological sample taken from the subject, including, but not limited to, any tissue, cell, or fluid (e.g., blood, plasma, serum, or urine) which can be analyzed for a trait of interest, such as the presence or amount of a nucleic acid (e.g., SORD mRNA), a protein (e.g., SORD protein), or sorbitol. In various embodiments, the biological sample is a plasma, serum, saliva, urine, or skin sample.

A “subject” as referred to herein, can be any mammal, such as humans. Animals of agricultural importance, such as bovine, equine, and porcine animals, are contemplated, as well as animals important as domestic pets, including canines and felines; animals important in research, including rodents and primates; and large endangered species and zoo animals such as primates, felines, giraffes, elephants, rhinos.

In various aspects, the method comprises treating the subject by administering to the subject a composition that comprises one or more aldose reductase inhibitors. In some embodiments, the aldose reductase inhibitor is Alrestatin, Epalrestat, Diepalrestat, Fidarestat, Imirestat, Lidorestat, Minalrestat, Ponalrestat, Ranirestat, Salfredin B₁₁, Sorbinil, Tolrestat, Zenarestat, or Zopolrestat. Aldose reductase inhibitors are reviewed in Expert Opin Ther Pat. 2019;29(3):199-213; Chatzopoulou et al., Expert Opin Ther Pat. 2012;22(11):1303-23 (incorporated by reference in their entirety).

In some embodiments, enzyme replacement therapy is employed, and a SORD peptide is administered to the subject. As such, the therapy supplements SORD peptide levels where endogenous SORD levels are inadequate or absent. An exemplary SORD peptide is provided in SEQ ID NO: 46. The disclosure contemplates use of a peptide that comprises at least 80%, at least 85%, at least 90%, at least 95%, or 100% identity to SEQ ID NO: 46.

In various embodiments, the method comprises administering to the subject a polynucleotide (e.g., an aldose reductase antisense oligonucleotide, a polynucleotide that encodes the SORD peptide/protein, an agent that blocks expression of a mutant SORD gene, and/or an agent that corrects the mutation in SORD gene). Polynucleotides are typically delivered to a host cell via an expression vector, which includes the regulatory sequences necessary for delivery and expression, although use of expression vectors are not required in the context of the disclosure. In some aspects, the constructs described herein include a promoter (e.g., cytomegalovirus (CMV) promoter or CB7 promoter), a protein coding region (optionally with non-coding (e.g. 3′-UTR) regions that facilitate expression), transcription termination sequences, and/or regulator elements sequences (e.g., Posttranscriptional Regulatory Element (WPRE), poly (A) element, origin of replication (pUC-ori) and/or ITR sequences (inverted terminal repeat)). In various aspects, the constructs described herein include one or more of vector features listed in Table 1. Vector features are also reviewed in Powell et al., Discov Med. 2015; 19(102): 49-57 (incorporated by reference in its entirety). For example, the Cre-loxP system may be utilized to express a peptide of interest (e.g., a SORD peptides, optionally in a specific tissue of interest). Expression vectors may be viral-based (e.g., retrovirus-, adenovirus-, or adeno-associated virus-based) or non-viral vectors (e.g., plasmids). Non-vector based methods (e.g., using naked DNA, DNA complexes, etc.) also may be employed. Optionally, the vector is a viral vector, such as a lentiviral vector or baculoviral vector, and in various preferred embodiments the vector is an adeno-associated viral vector (AAV). The expression vector may be based on any AAV serotype, including AAV-1, AAV-2, AAV-3, AAV-4, AAV-5, AAV-6, AAV-7, AAV-8, AAV-9,AAV-10, AAV-11, AAV-12, or AAV-13. Polynucleotides also may be delivered via liposomes, nanoparticles, exosomes, microvesicles, hydrodynamic-based gene delivery, or via a “gene-gun.”

TABLE 1
Vector feature elements

| | | | Post- | | |
|---|---|---|---|---|---|
| | Neuronal | Introns | transcriptional | | |
| Ubiquitous | specific | (enhanced | regulatory | Polyadenylation | Bacterial |
| Promoters | promoter | expression) | elements | signal enhancers | resistance |
| CMV, Cba, | NFL, | b-Glob, | HPRE, WPRE | hGH, bGHpA, | Ampicillin |
| CAG, CBh, | NFH, | MVM, I.IX, | | SPA, SV40 late | Kanamycin |
| EF1-α, | synapsin, | adenovirus | | | |
| PGK, UBC | CaMKII, | SD/ | | | |
| | Hb9, | immuno- | | | |
| | MeCP2 | globulin SA, | | | |
| | | SV40 late | | | |
| | | SD/SA | | | |

Titers of AAV to be administered in methods of the disclosure will vary depending, for example, on the particular AAV, the mode of administration, the treatment goal, the individual, and the cell type(s) being targeted, and may be determined by methods known in the art. Titers of AAV may range from about 1×10⁶, about 1×10⁷, about 1×10⁸, about 1×10⁹, about 1×10¹⁰ about 1×10¹¹, about 1×10¹², about 1×10¹³ to about 1×10¹⁴ or more DNase resistant particles (DRP) per ml. Dosages may also be expressed in units of viral genomes (vg).

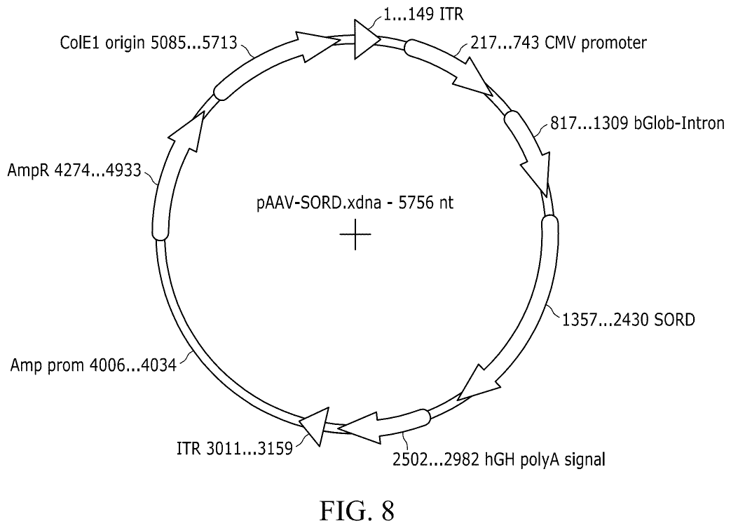

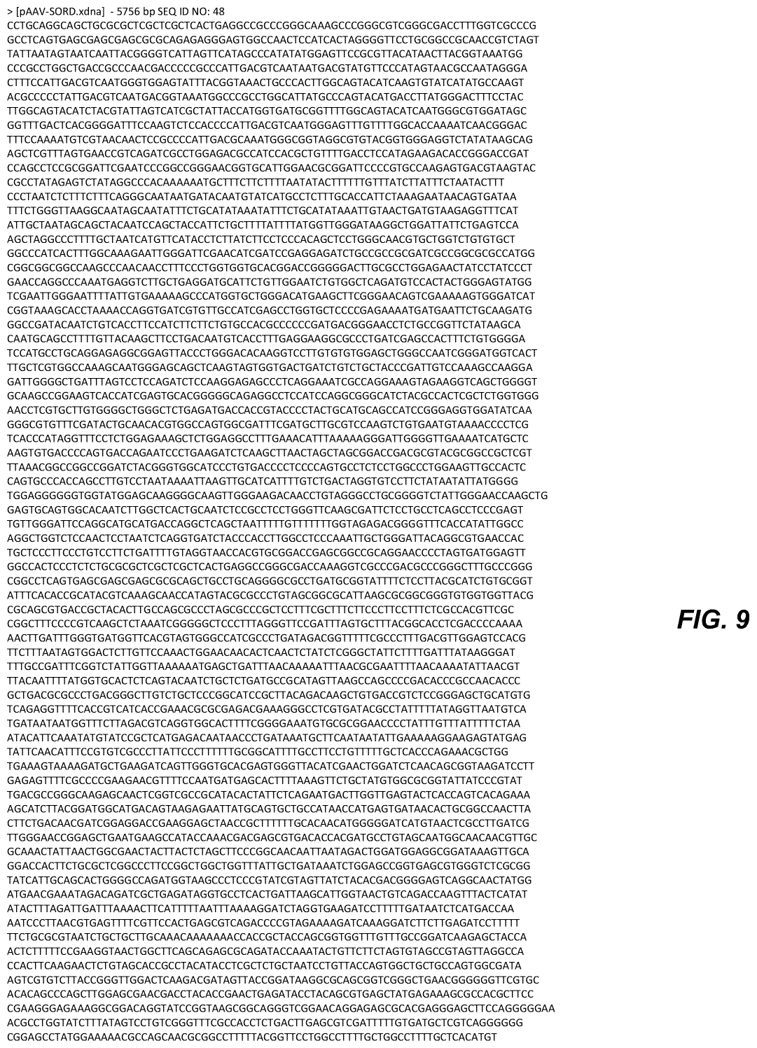

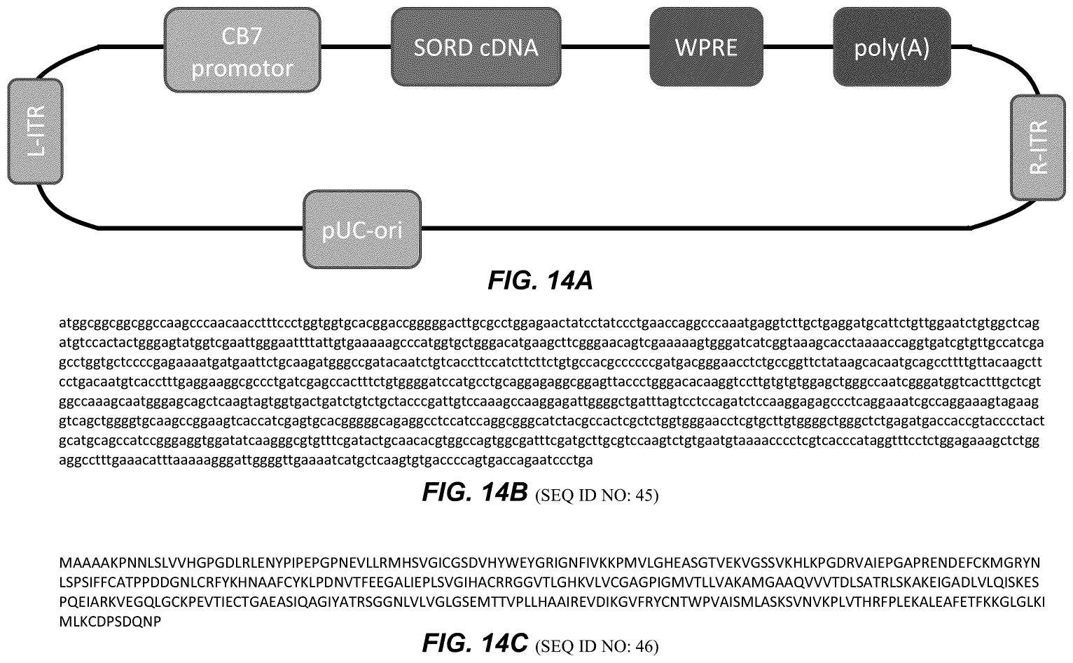

In various embodiments, a polynucleotide that encodes a SORD peptide is administered to the subject. The amino acid sequence of SORD is provided as SEQ ID NO: 46 (FIG. 14C, NCBI Reference Sequence: NP_003095.2). The polynucleotide used in the method optionally encodes the amino acid sequence of SEQ ID NO: 46 or a sequence that is at least 80%, at least 85%, at least 90%, at least 95%, or 100% identical to the amino acid sequence of SEQ ID NO: 46 (which retains the function of SORD). Optionally, the polynucleotide comprises SEQ ID NO: 45 (FIG. 14B) or a sequence that is at least 80%, at least 85%, at least 90%, at least 95%, or 100% identical to the polynucleotide sequence of SEQ ID NO: 45 (and which encodes SORD). Exemplary expression vectors comprising a polynucleotide encoding the SORD peptide are illustrated in FIGS. 8 and 14A. The polynucleotide, in at least one aspect of the disclosure, comprises the nucleic acid sequence shown in FIG. 9 (SEQ ID NO: 1), which corresponds to the sequence of an AAV vector comprising a polynucleotide encoding SORD.

In various embodiments, the method comprises administering to the subject an agent that blocks expression of a mutant SORD gene. An agent that blocks expression of a mutant SORD gene refers to an agent that interferes with expression of a SORD gene so that SORD gene expression and/or SORD protein levels are reduced compared to basal/wild-type levels. It will be appreciated that “blocking” expression of a mutant SORD gene does not require 100% abolition of expression and SORD production; any level of reduced expression of aberrant SORD may be beneficial to a subject. Exemplary agents include, but are not limited to, antisense oligonucleotides (ASO), short hairpin RNA (shRNA), small interfering RNA (siRNA), or micro RNA (miRNA).

In various embodiments, the method comprises administering to the subject an aldose reductase antisense oligonucleotide which targets the aldose reductase sequence such that expression of the enzyme is blocked. An aldose reductase, aldo-keto reductase family 1 member B (AKRIB1), is encoded by SEQ ID NO: 48 (NCBI Reference Sequence: NM_001628). An aldose reductase antisense oligonucleotide interferes with expression of an aldose reductase gene (AKR1B1), so that AKR1B1 gene expression and/or aldose reductase protein levels are reduced compared to basal/wild-type levels. It will be appreciated that “blocking” expression of an aldose reductase gene (the AKR1B1 gene) does not require 100% abolition of expression and aldose reductase production; any level of reduced expression of aldose reductase may be beneficial to a subject. For example, in various aspects, the aldose reductase antisense oligonucleotide that reduces the expression of aldose reductase. An ASO is a single-stranded deoxyribonucleotide, which is complementary to an mRNA target sequence. In various aspects, the aldose reductase antisense oligonucleotide targets an exonic or intronic sequence of the aldose reductase gene.

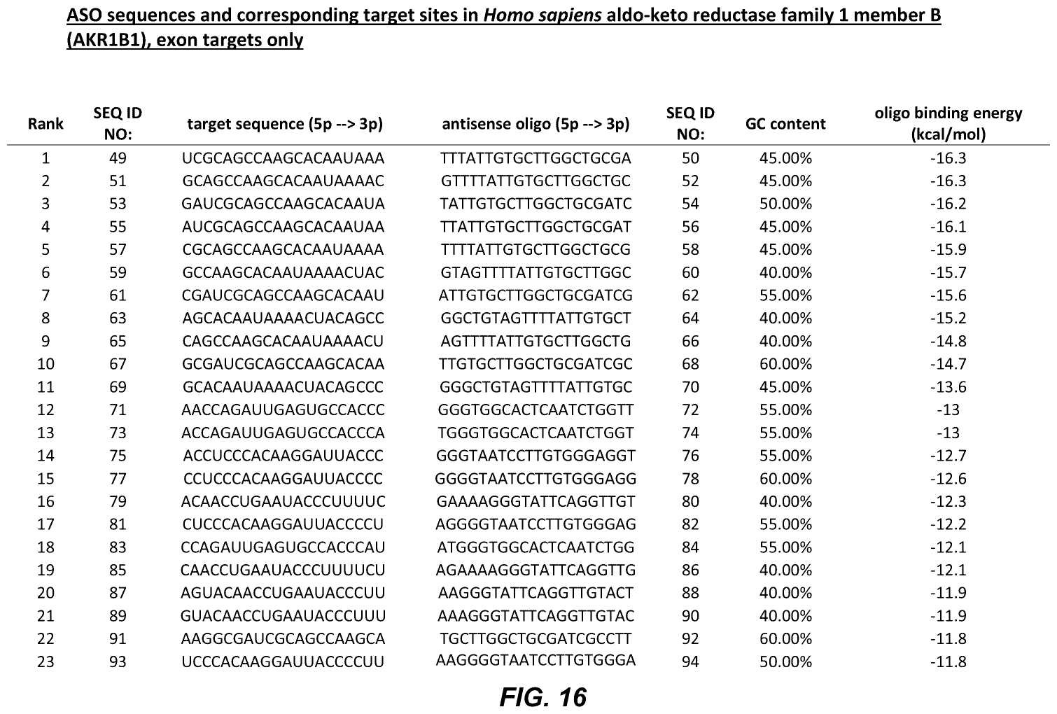

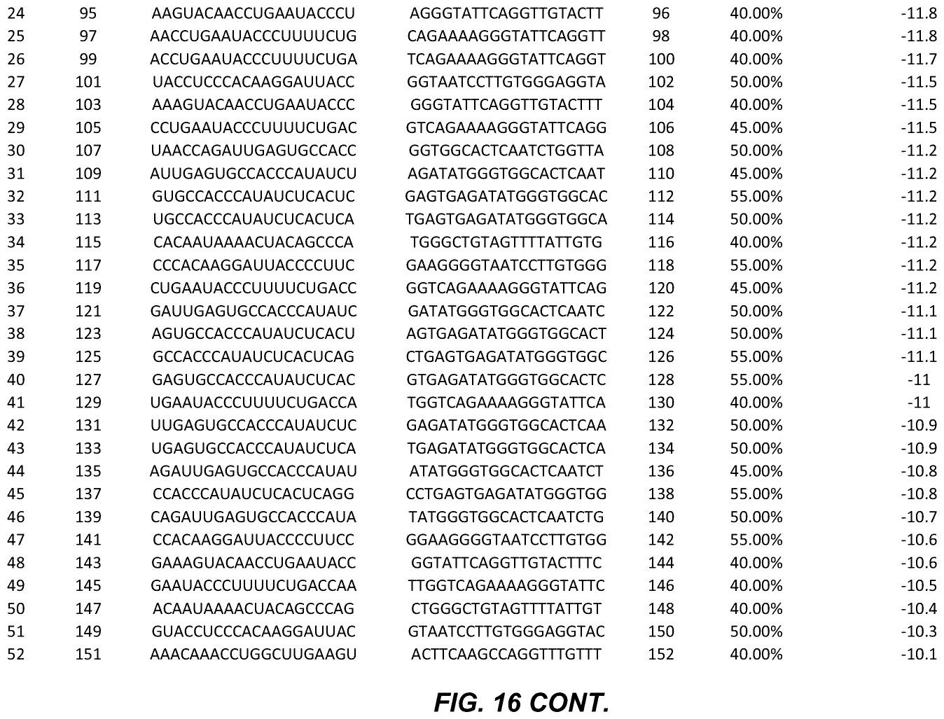

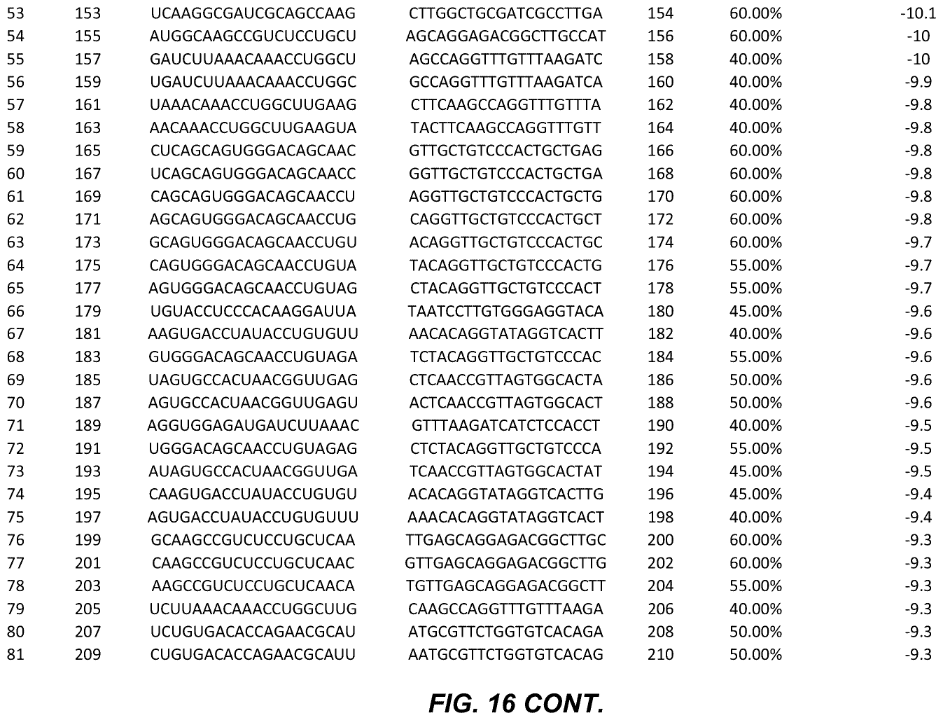

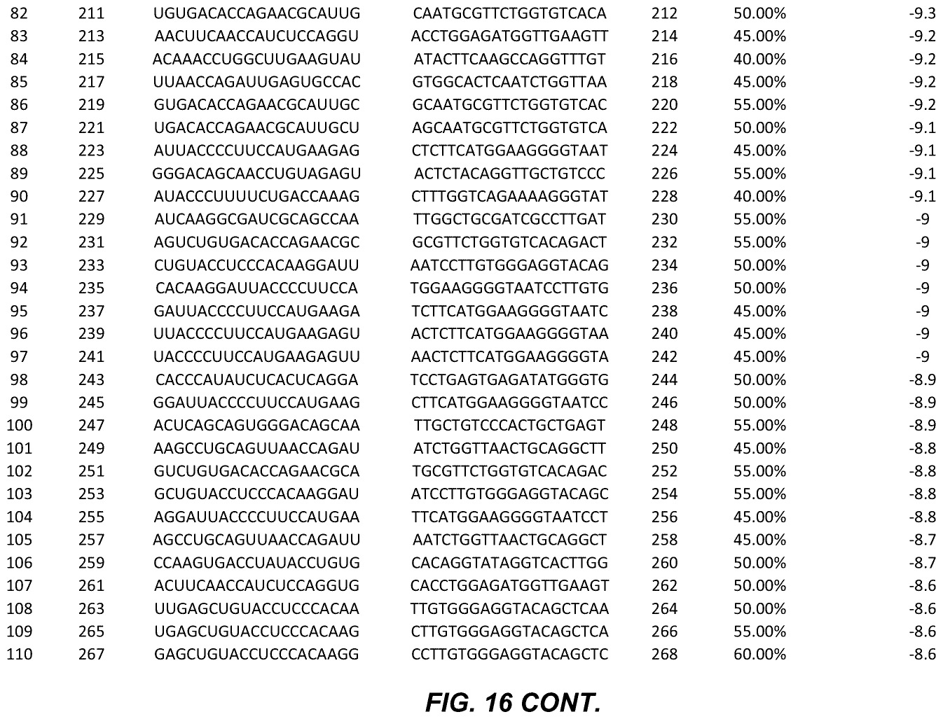

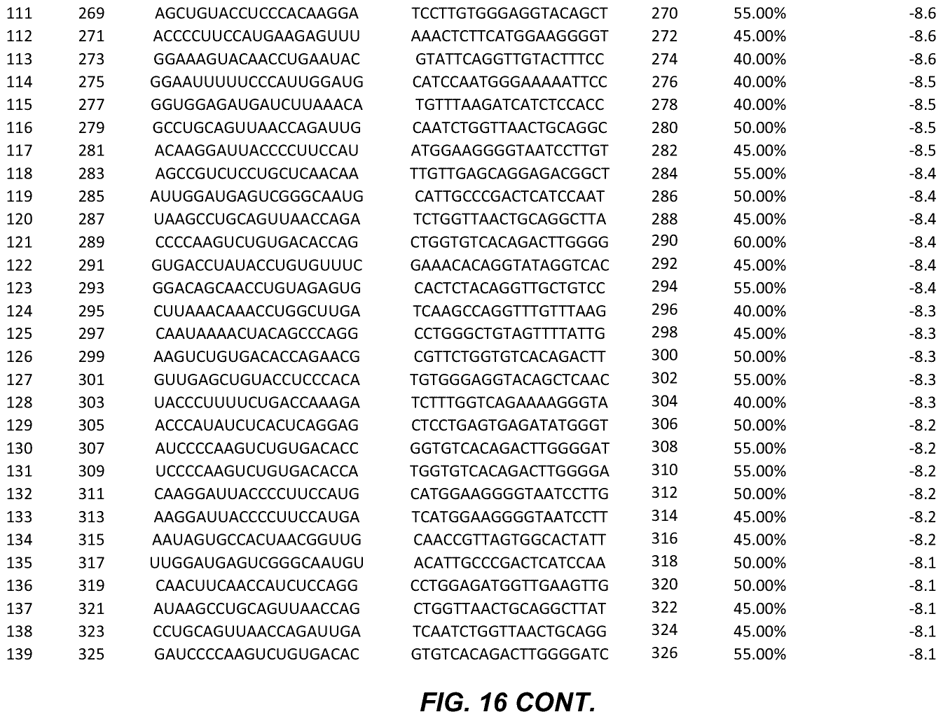

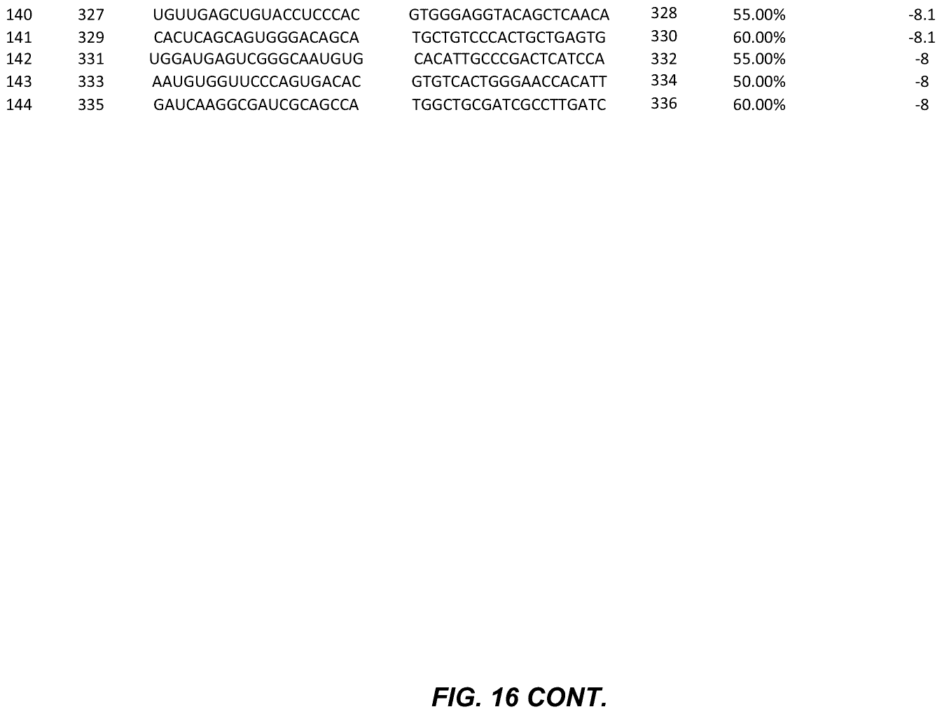

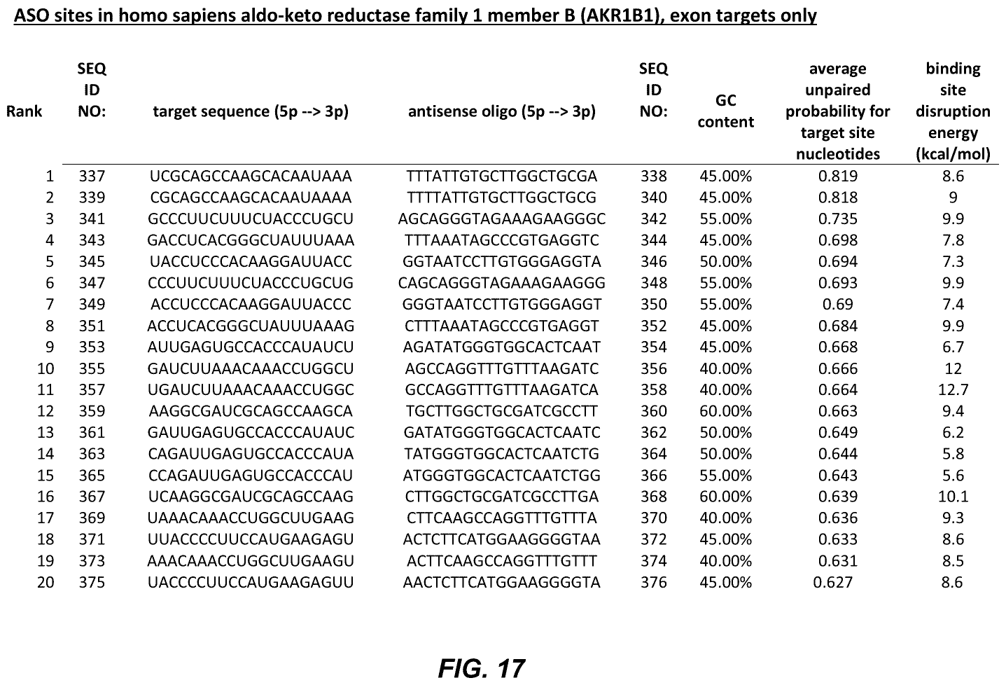

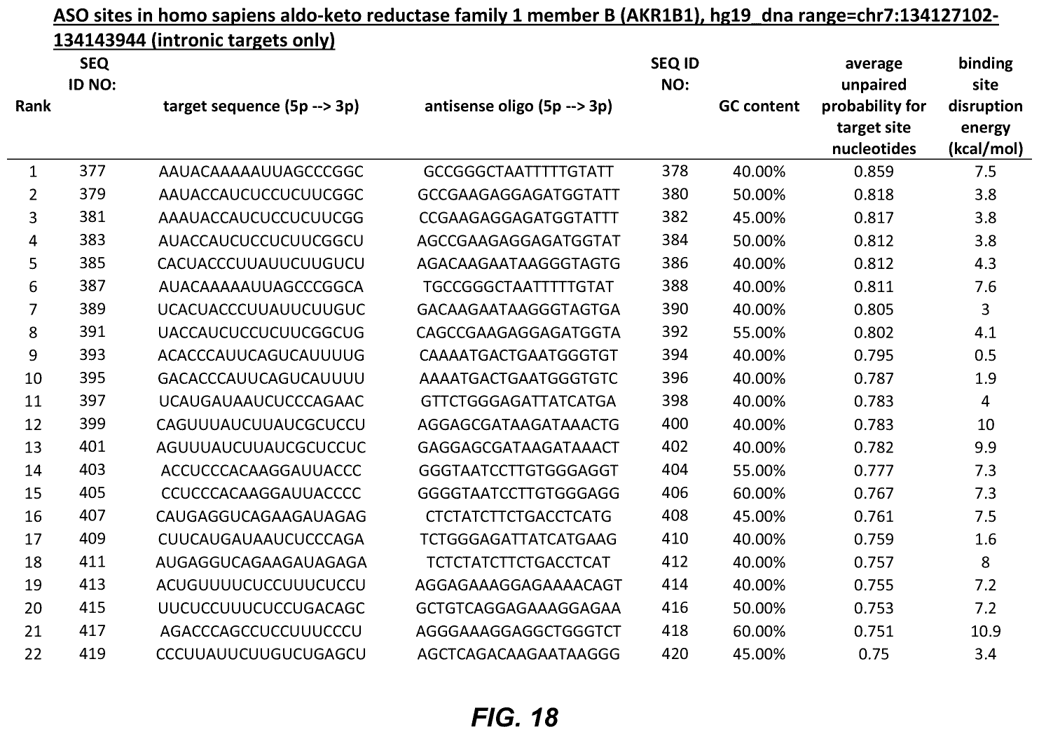

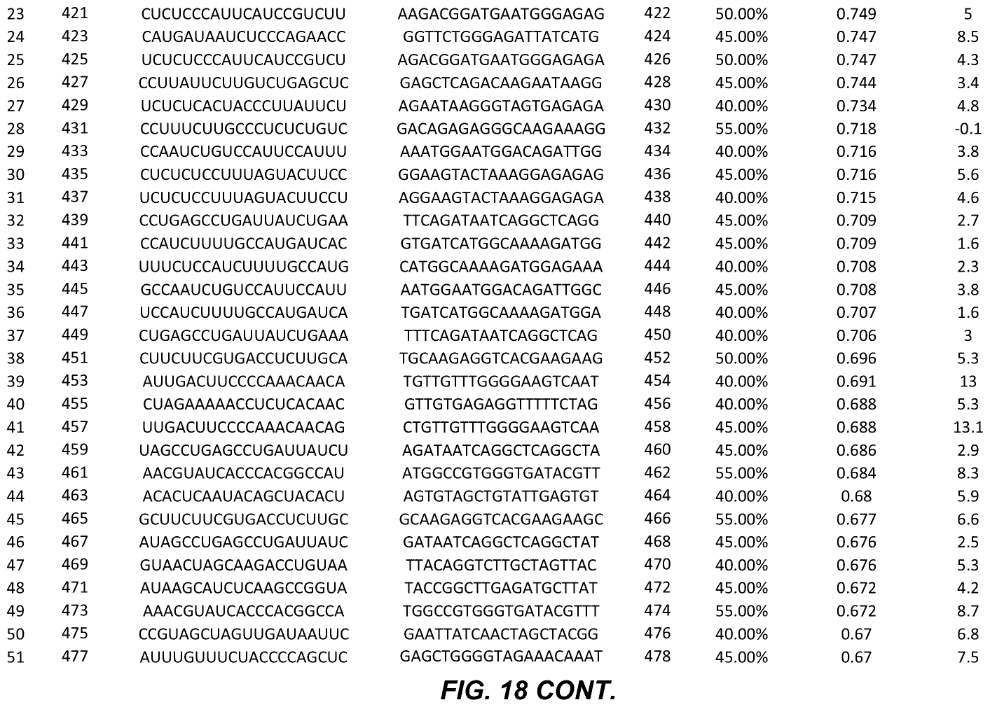

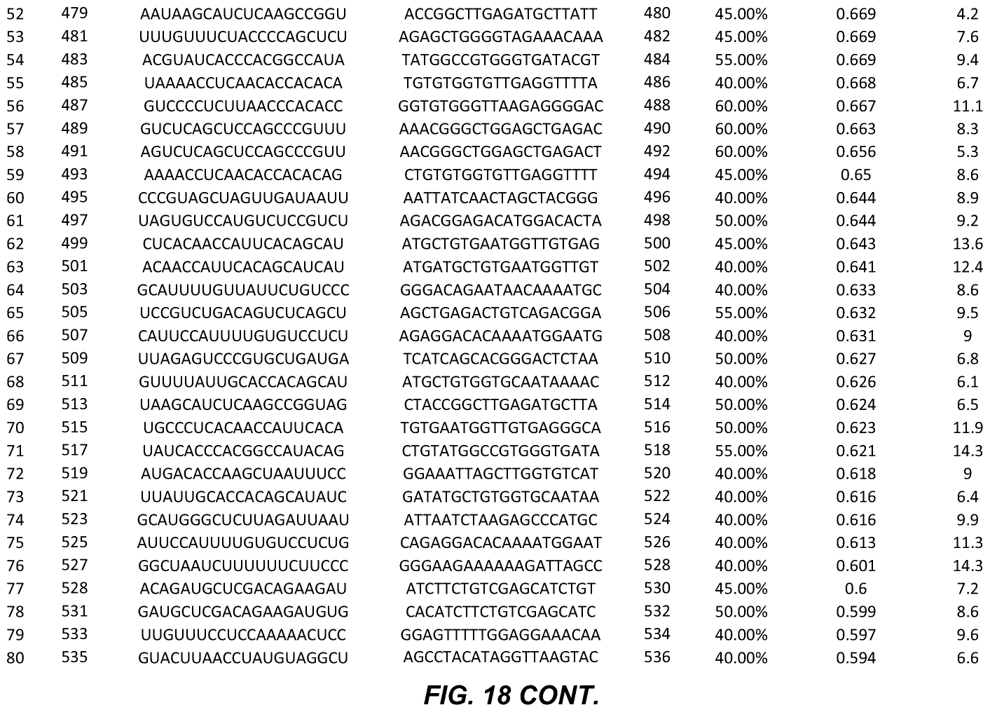

In an exemplary method for identifying ASO sequences targeting aldose reductase, the following criteria were used: A) sequences targeting aldose reductase (AKR1B1) were selected which contained<=40% GC or <=60% GC content; B) sequences containing GGGG nucleotides were excluded; C) sequences with an average unpaired probability for target site nucleotides>=0.5 were selected; D) for each peak in the accessibility profile that was above the threshold probability of 0.5, all sites targeted to the same peak were ranked by their average unpaired probability (the higher the better) and at most n sites are selected for each peak, where n is determined by max width of peak/site length. Exemplary agents satisfying these criteria are provided in Table 2. Additional exemplary ASO sequences and filter criteria are shown in FIGS. 16-18.

TABLE 2
ASO sequences targeting AKR1B1/aldose reductase.

| Starting | Ending | | SEQ | | SEQ | | Average | Binding |
|---|---|---|---|---|---|---|---|---|
| target | target | | ID | | ID | GC | unpaired | site |
| position | position | Target Seq (5′-3′) | NO | antisense oligo (5′-3′) | NO | content | probability | disruption |
| 475 | | AGGUGGAGAUGAUCUUAAAC | 21 | GTTTAAGATCATCTCCACCT | 22 | 40.00% | 0.594 | 12.5 |
| 476 | 495 | GGUGGAGAUGAUCUUAAACA | 23 | TGTTTAAGATCATCTCCACC | 24 | 40.00% | 0.592 | 12.6 |
| 484 | 503 | UGAUCUUAAACAAACCUGGC | 25 | GCCAGGTTTGTTTAAGATCA | 26 | 40.00% | 0.66 | 12.5 |
| 490 | 509 | UAAACAAACCUGGCUUGAAG | 27 | CTTCAAGCCAGGTTTGTTTA | 28 | 40.00% | 0.633 | 9.2 |
| 534 | 555 | CAGGUGGAGAUGAUCUUAAACA | 29 | TGTTTAAGATCATCTCCACCTG | 30 | 40.90% | 0.584 | 12.6 |
| 545 | 566 | GAUCUUAAACAAACCUGGCUUG | 31 | CAAGCCAGGTTTGTTTAAGATC | 32 | 40.90% | 0.611 | 10.3 |
| 548 | 569 | CUUAAACAAACCUGGCUUGAAG | 33 | CTTCAAGCCAGGTTTGTTTAAG | 34 | 40.90% | 0.646 | 9.5 |
| 567 | 588 | AAGUAUAAGCCUGCAGUUAACC | 35 | GGTTAACTGCAGGCTTATACTT | 36 | 40.90% | 0.584 | 7.7 |
| 739 | 760 | UCAAGGCGAUCGCAGCCAAGCA | 37 | TGCTTGGCTGCGATCGCCTTGA | 38 | 59.10% | 0.669 | 10 |
| 741 | 762 | AAGGCGAUCGCAGCCAAGCACA | 39 | TGTGCTTGGCTGCGATCGCCTT | 40 | 59.10% | 0.694 | 9.3 |
| 1025 | 1046 | ACCUGUGUUUCUUGCCUCAUUU | 41 | AAATGAGGCAAGAAACACAGGT | 42 | 40.90% | 0.641 | 5.5 |

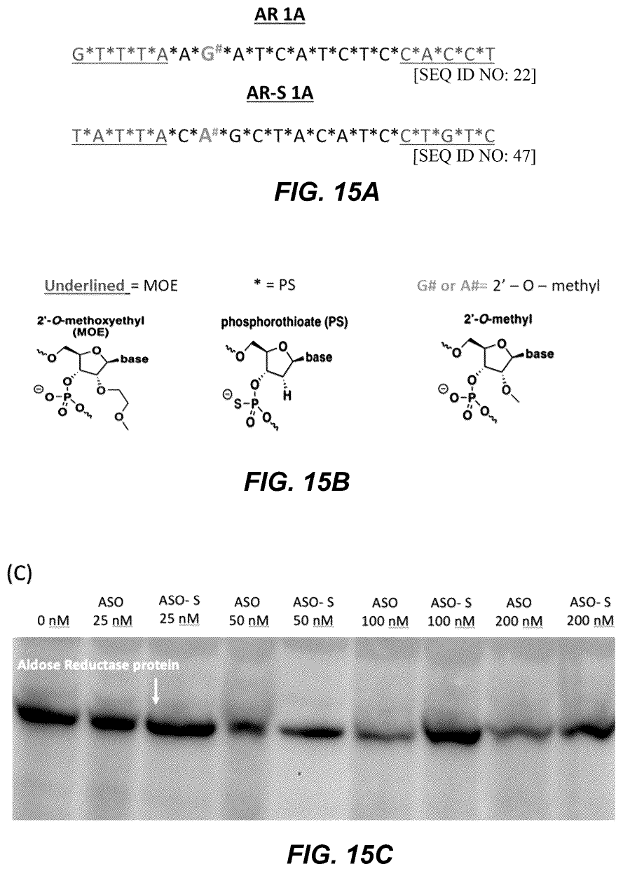

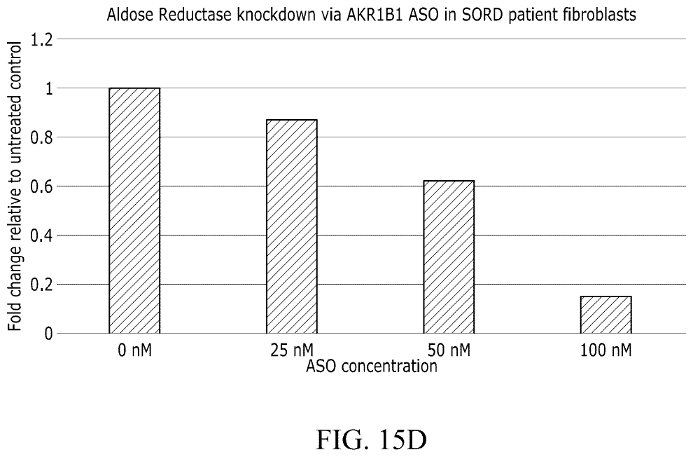

In various embodiments, the nucleotide backbone of ASO sequences are modified to a chimeric or gapmer design to reduce gene expression when compared to basal/wild-type levels. In various embodiments, a gapmer design requires a designation of 3-5 nucleotides on each end of the antisense oligonucleotide sequence to harbor modifications in the ribose sugar moiety resistant to RNase H recognition and other nucleases, while all other nucleotides contain an RNase H compatible modification. RNase H is responsible for cleaving RNA-DNA duplexes such as those formed between aberrant mRNA transcripts and synthetically designed DNA antisense oligonucleotides. In various embodiments, the modification to the ASO sequences includes, but is not limited to Phosphorothioate (PS)—RNase H recognizable, phosphorodiamidate morpholino (PMO)-RNase H resistant, 2′-O-methyl-RNase H resistant, 2′-O-methoxyethyl (MOE)—RNase H resistant, locked Nucleic Acid (LNA)—RNase H resistant, ethylene-bridged nucleic acid (ENA)—RNase H resistant, or (S)-constrained ethyl (cEt)—RNase H resistant. Exemplary modification of an ASO sequence is shown in FIGS. 15A-15B. Modifications to ASO sequences are reviewed in Scoles et al., Neurol Genet. 2019;5(2):e323. (incorporated by reference in its entirety).

In various aspects, the method employs RNA interference (RNAi) to regulate expression of SORD. The RNAi pathway is summarized in Duan (Ed.), Section 7.3 of Chapter 7 in Muscle Gene Therapy, Springer Science+Business Media, LLC (2010). Suitable agents include, e.g., siRNA, miRNA, and shRNA. A shRNA/Hairpin Vector is an artificial RNA molecule (nucleotide) with a tight hairpin turn that can be used to silence target gene expression via RNAi. shRNA is an advantageous mediator of RNAi in that it has a relatively low rate of degradation and turnover, but it often requires use of an expression vector. In exemplary aspects, the disclosure includes the production and administration of an AAV vector expressing one or more shRNAs targeting SORD. The expression of shRNAs is regulated by the use of various promoters. In various aspects, polymerase II promoters, such as U6 and H1, and polymerase III promoters are used. In some aspects, U6 shRNAs are used. It will be appreciated that RNAi also may be used to downregulate (i.e., block) expression of aldose reductase (e.g., AKR1B1); as such, the disclosure contemplates sue of siRNA, miRNA, and shRNA which targets aldose reductase intronic or extronic sequences to block the expression of aldose reductase.

Traditional small/short hairpin RNA (shRNA) sequences are usually transcribed inside the cell nucleus from a vector containing a Pol III promoter such as U6. The endogenous U6 promoter normally controls expression of the U6 RNA, a small RNA involved in splicing, and has been well-characterized (Kunkel et al., Nature. 322(6074):73-7 (1986); Kunkel et al., Genes Dev. 2(2):196-204 (1988); Paule et al., Nucleic Acids Res. 28(6):1283-98 (2000)). The disclosure includes both murine and human U6 or H1 promoters. The shRNA containing the sense and antisense sequences from a target gene connected by a loop is transported from the nucleus into the cytoplasm where Dicer processes it into siRNAs.

In some aspects of the disclosure, an agent that corrects the mutation in the SORD gene is employed. An agent that corrects the mutation in SORD gene refers to an agent capable of modifying the SORD coding sequence or a regulatory element and/or non-coding region associated with the SORD gene to achieve a desired change in the sequence. In various aspects, genome editing may be used to replace part or all of the SORD gene sequence or alter SORD protein expression levels. In various embodiments, the agent may comprise components employed in genome-editing techniques, such as designer zinc fingers, transcription activator-like effectors nucleases (TALENs), or CRISPR-Cas (clustered regularly interspaced short palindromic repeats-CRISPR associated) systems. An exemplary agent for use in the method of the disclosure is, DNA encoding Cas9 molecules and/or gRNA molecules. Cas9 and gRNA can be present in a single expression vector or separate expression vectors. Adenoviral delivery of the CRISPR/Cas9 system is described in Holkers et al., Nature Methods (2014), 11(10):1051-1057 which is incorporated by reference in its entirety.

Other publications describing the CRISPR systems and Cas9 include the following: Cong et al. Science (2013) 339:819-23; Jinek et al., Elife. (2013) 2:e00471; Lei et al. Cell (2013) 152:1173-1183; Gilbert et al. Cell (2013) 154:442-51; Lei et al. Elife (2014) 3:e04766; Perez-Pinela et al. Nat Methods (2013) 10:973-976; Maider et al. Nature Methods (2013) 10, 977-979; U.S. Pat. Nos. 8,697,359; 8,771,945; 8,795,965; 8,865,406; 8,871,445; 8,889,356; 8,895,308; 8,906,616; 8,932,814; 8,945,839; 8,993,233; 8,999,641; U.S. Application Publication No. 2014/0068797; and International Patent Publication No. WO 2014/197568, all incorporated by reference in their entirety.

In some embodiments, CRISPR/Cas9 multiplexing may be used to target multiple genomic loci wherein two or more guide RNAs are expressed as described in CRISPR 101: A Desktop Resource (1^{st} Edition), Addgene, January 2016 which is incorporated by reference in its entirety.

The terms “treating” or “treatment” refer to reducing or ameliorating inherited neuropathy and/or associated disorders and/or symptoms associated therewith. These terms include reducing or delaying the frequency of occurrence or recurrence of the neuropathy or symptoms associated therewith (i.e., lengthening the period of remission in a patient who had suffered from the disorder), as well as reducing the severity of the disorder or any symptoms associated therewith. It is appreciated that, although not precluded, “treating” or “treatment” of a disorder or condition does not require that the disorder, condition, or symptoms associated therewith be completely eliminated.

A dose of an active agent (e.g., an aldose reductase inhibitor, an aldose reductase antisense oligonucleotide, a polynucleotide that encodes a SORD peptide, a SORD peptide, an agent that blocks expression of a mutant SORD gene, or an agent that corrects the mutation in SORD gene) will depend on factors such as route of administration (e.g., local vs. systemic), patient characteristics (e.g., gender, weight, health, side effects), the nature and extent of the inherited neuropathy or associated disorder, and the particular active agent or combination of active agents selected for administration.

The active agents described herein are provided in a composition (e.g., a pharmaceutically-acceptable composition) which may contain formulation components suitable for administration to a subject, as well as additional therapeutic agents. Suitable methods of administering a physiologically-acceptable composition, such as a pharmaceutical composition comprising an agent described herein, are well known in the art. In various aspects, more than one route can be used to administer one or more of the agents disclosed herein. A particular route can provide a more immediate and more effective reaction than another route. For example, in certain circumstances, it will be desirable to deliver the composition orally; through injection or infusion by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means; by controlled, delayed, sustained or otherwise modified release systems; by implantation devices; using nanoparticles; or as a conjugate.

It is contemplated the two or more active agents described herein may be administered as part of a therapeutic regimen. Alternatively or in addition, one or more of the active agents may be administered with other therapeutics as part of a therapeutic regimen. The active agent(s) may be administered as a monotherapy or as a combination therapy with other treatments administered simultaneously or metronomically. The term “simultaneous” or “simultaneously” refers to administration of two agents within six hours or less (e.g., within three hours or within one hour each other). In this regard, multiple active (or therapeutic) agents may be administered the same composition or in separate compositions provided within a short period of time (e.g., within 30 minutes). The term “metronomically” means the administration of different agents at different times and at a frequency relative to repeat administration. Active agents need not be administered at the same time or by the same route; preferably, in various embodiments, there is an overlap in the time period during which different active agents are exerting their therapeutic effect. Additional aspects and details of the disclosure will be apparent from the following examples, which are intended to be illustrative rather than limiting.

### EXAMPLES

### General Methods

### Families

All families provided written informed consent to participate in the study. The study protocol was approved by the institutional review board of giving institutions. All patients were clinically evaluated by neurologists.

### Whole Exome and Sanger Sequencing

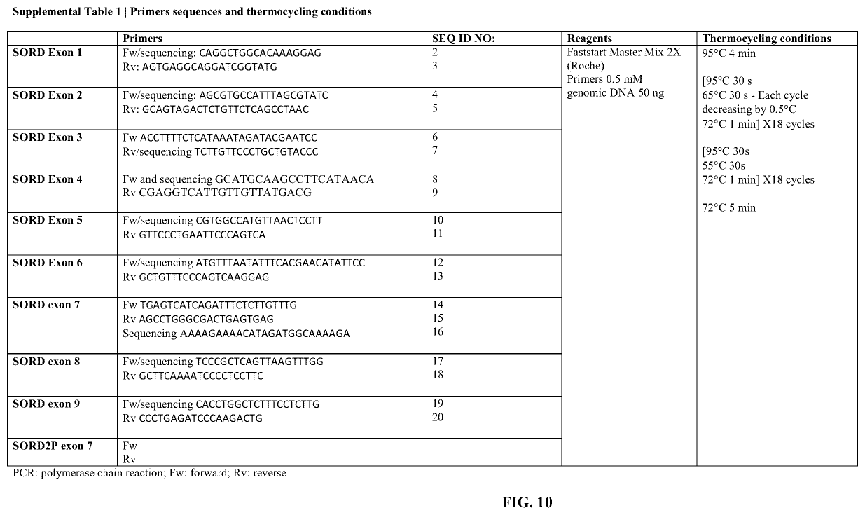

Whole exome sequencing was performed in index individuals from sporadic and recessive CMT and dHMN families. The SureSelect Human All Exon 50 MB Kit (Agilent) was used for in-solution enrichment, and the HiSeq 2500 instrument (Illumina) was used to produce about 120 bp paired-end sequence reads. The Burrows-Wheeler aligner, and Freebayers were used to align sequence reads and call variants. Final data were uploaded into GENESIS software for analysis. A filtering approach to search for families sharing the same homozygous variants were applied across all exomes in the database. Sanger sequencing, performed by Eurofins Genomics, confirmed segregation of the SORD variants. Polymerase chain reaction (PCR) was carried out in the Veriti Thermocycler (Applied Biosystem) and Platinum Taq (ThermoFisher) was used to amplify the regions containing the target mutations. The following primers were used to target specifically SORD and not SOR2P (FIG. 10).

### Fibroblasts Culture

Fibroblasts were obtained from patients and cultured in Dulbecco's Modified Eagle Medium (ThermoFisher) supplemented with 10% fetal bovine serum (FBS), penicillin and streptomycin (Gibco). Cells were maintained in 5% CO₂ at 37° C. in a humidified incubator. Asynchronous cell cultures were grown to approximately 80% confluency and treated with epalrestat (100 μM), ranirestat (10 μM) or DMSO for 72 hours. Media containing the drugs or DMSO were changed every 24 hours.

### Western Blot

Fibroblasts were lysed in RIPA buffer (ThermoFisher) containing protease inhibitor (Roche) and sonicated for 5 minutes with the Bioruptor sonication device (Diagenode). Cell lysates were centrifuged at 13,000×g for 10 minutes at 4° C., and the supernatant was collected for protein quantification (Pierce BCA Protein Assay Kit). 30 μg of protein sample was mixed with Bolt LDS Sample Buffer and Sample Reducing Agent (ThermoFisher) and heated at 90° C. for 5 min. Samples were loaded on Bolt 4-12% Bis-Tris Plus mini-gel followed by transfer into a nitrocellulose membrane (Bio-Rad). Membrane was blocked with 5% non-fat milk and incubated with anti-SORD (ab189248, Abcam) antibody for 2 hours, washed with TBS containing 0.01% Tween 20 (Bio-Rad) and incubated with a secondary anti-rabbit antibody (Cell Signaling). Membrane was subsequently incubated with GAPDH primary antibody (Santa Cruz) and secondary anti-mouse antibody (Cell Signaling). Chemoluminescence detection was performed with the SuperSignal West Pico PLUS Chemiluminescent Substrate and imaged with the FluorChem E (ProteinSimple).

### Sorbitol Measurement

Fibroblasts were collected and lysed as described in Western blot section in the absence of proteinase inhibitor. Sorbitol determination in human fibroblast lysates was performed in ultra-performance liquid chromatography-tandem mass spectrometry (UPLC-MS/MS) (Waters Acquity UPLC & TQD mass spectrometer—Waters, Milford, MA, USA). Fibroblasts were collected and lysed as described in Western blot section in the absence of proteinase inhibitor. Proteinase inhibitor contains high concentration mannitol, which is a sorbitol enantiomer, and can interfere with UPLC-MS/MS sorbitol determination. Lysate samples underwent protein precipitation with Acetonitrile (1:5), ten-time dilution with Acetonitrile-water (50/50) and clean up on Oasis HLB cartridges (10 mg/1 ml), before injection in UPLC (3 μL). UPLC conditions: column, BEH Amide 1.7 μm (2.1˜100 mm) at 88° C., eluent A, Acetonitrile 90%-water 5%-Isopropanol 5%, eluent B, Acetonitrile 80%-water 20%, gradient elution 0 min., 100% A, 3.6 min. 100% B, flow rate, 0.45 ml/min. The retention time of sorbitol was 2.7 min. MS/MS conditions: interface, Electrospray interface in negative ion mode, Multiple Reaction Monitoring acquisition, m/z 180.9→88.9 (CV 24, CE 15).

For fasting sorbitol level testing, blood was collected after overnight fasting (last meal the evening before) in serum separator tubes. Samples were centrifuged at 500 g for 10 min. Serum was separated and frozen within an hour from blood collection. Sorbitol level was tested by UPLC using a method adapted from Li et al. Biochem Biophys Res Commun. 2009 Oct. 2;387(4):778-83. Conditions were as follows: column, BEH Amide 1.7 um maintained at 25° C. (instead of 45° C.); eluent A, 10 mM ammonium acetate pH10; eluent B, Acetonitrile. Flow rate, 0.6 ml/min with the 20 same gradient. The retention time of Sorbitol was 6.0 min. MS/MS conditions were the same of fibroblast analysis. Serum samples underwent protein precipitation with cold Methanol (1:5), five time dilution with Acetonitrile-water (50/50) and clean up on Oasis HLB cartridges (10 mg/1 ml), before injection in UPLC (3 μL). Calibration curve was done in serum in sorbitol concentration range 0.1-20 mg/L.

### Drosophila Stocks and Genetics

Unless specified, all flies were kept on cornmeal-molasses-yeast medium at 25° C., 65% humidity, with 12 h light/12 h dark cycles. The following fly strains used in this study were obtained from Bloomington Drosophila Stock Center: elav^{C155}-GAL4, GMR-GAL4, Sdh2^{MB01265}, UAS-SdhIRNAi, and UAS-Sdh2RNAi.

### Drug Feeding

Epalrestat or ranirestat was dissolved in dimethyl sulfoxide (DMSO) to achieve a stock concentration of 10 mg/ml, and then mixed into 10 ml fly food at a final concentration of 80 μg/ml. Equal amount of DMSO was mixed into the fly food as control. The vials were dried at room temperature for 12 h before feeding.

### Drosophila Lifespan Assay and Negative Geotaxis Assay

For lifespan assay, 100 newly enclosed female flies of each group were collected and placed in vials of 20 individuals. Flies were transferred into new vials every 2 days and the number of dead flies was counted. Survival data was plotted using Kaplan-Meier plot and compared between groups using log-rank test. For negative geotaxis behavior assay, 10 age-matched female flies were placed in a vial marked with a black line drawn horizontally 8 cm above the bottom. Flies were given 60 min to fully recover from CO₂ anesthesia, and were gently tapped onto the bottom and given 10 s to climb. Flies that crossed the 8 cm line were counted. For each vial, this assay was repeated 10 times, and 10 independent vials of each group (a total 100 flies per group) were tested. To minimize observer-expectancy bias, this assay was performed with the examiner masked to the group assignment.

### Drosophila Brain Dissection, Immunostaining, and Confocal Microscopy

Brain dissection and staining were carried out as previously described (Brazill et al., J Vis Exp. 2018;(138)). Briefly, fly brains were dissected in phosphate-buffered saline (PBS, pH 7.4), fixed in 4% formaldehyde for 10 min, and washed in PBTX (PBS containing 0.4% v/v Triton X-100) for 3 times (15 mins each). Brains were then incubated with primary mouse anti-BRP antibody (nc82, Developmental Studies Hybridoma Bank) at 1:250 dilution in 0.4% PBTX with 5% normal goat serum at 4° C. overnight with gentle shaking. After that, brains were incubated with Cy3-conjugated anti-mouse secondary antibody (Rockland) and Cy5-conjugated anti-HRP (Jackson ImmunoLab) at 1:250 dilution at 4° C. overnight with gentle shaking, followed by 4′,6-diamidino-2-phenylindole (DAPI, 1:300, Invitrogen) staining at room temperature for 10 min. Samples were mounted on glass slides with VECTASHIELD Antifade Mounting Medium (Vector Laboratories Inc.). Fly brain slides were imaged using an Olympus IX81 confocal microscope with 60×oil immersion objective lens with a scan speed of 8.0 μs per pixel and spatial resolution of 1024×1024 pixels. Images were processed and analyzed using FluoView 10-ASW (Olympus).

Additional aspects and details of the disclosure will be apparent from the following examples, which are intended to be illustrative rather than limiting.

### Example 1: Identification of DNA Variants in CMT Using GENESIS Analysis

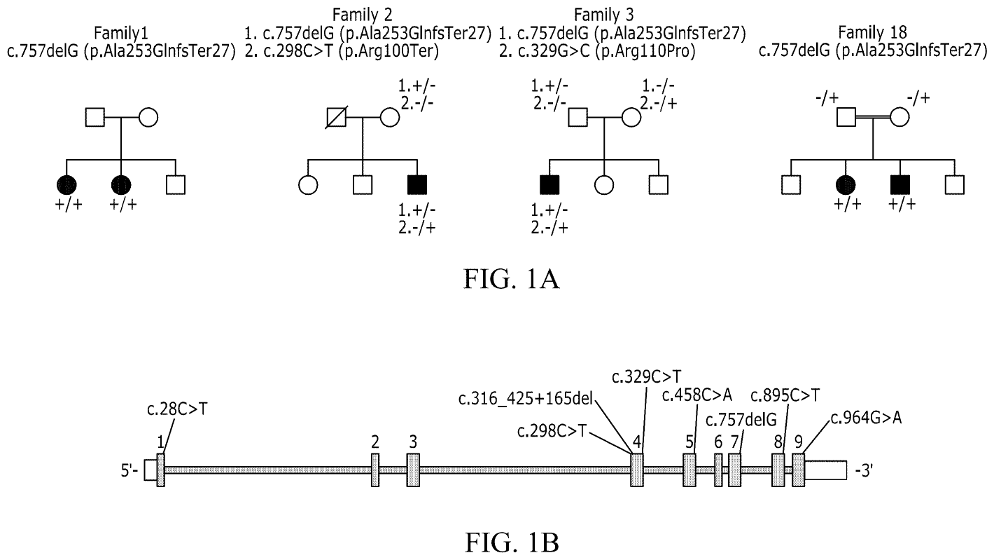

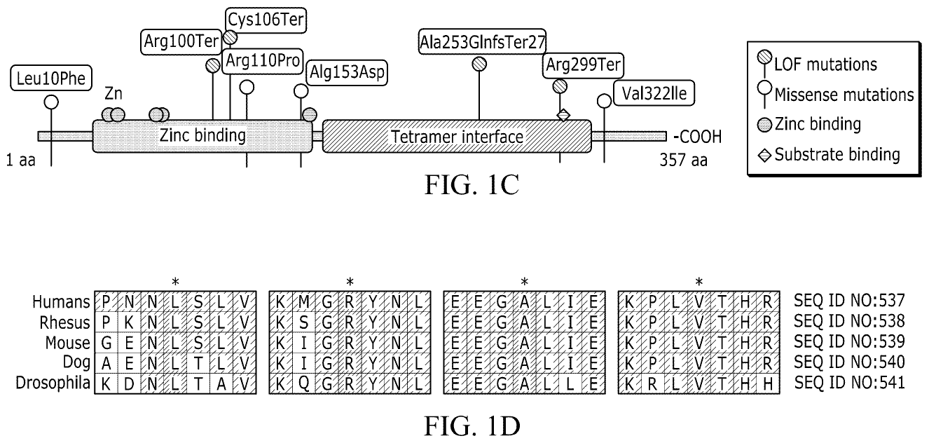

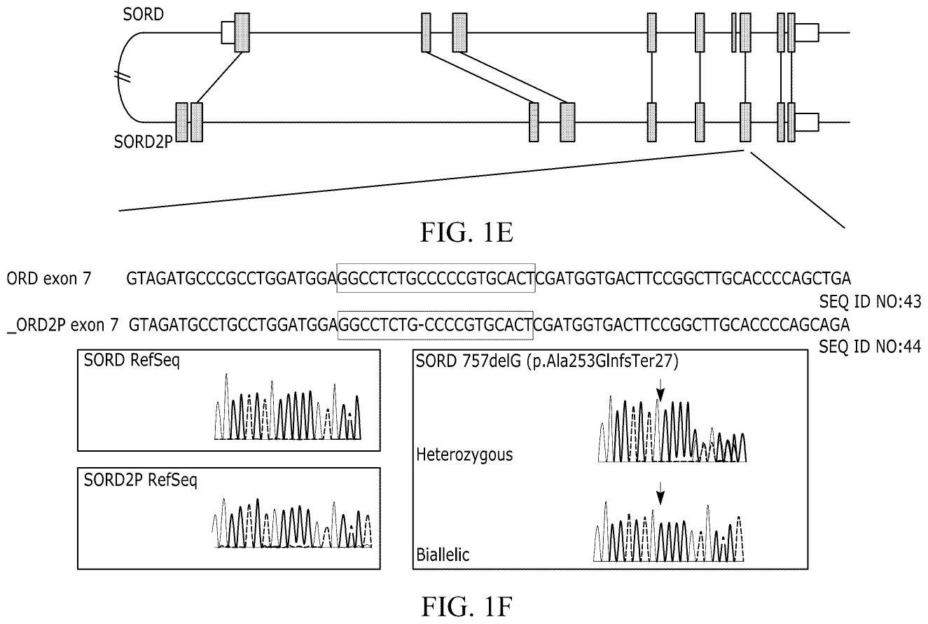

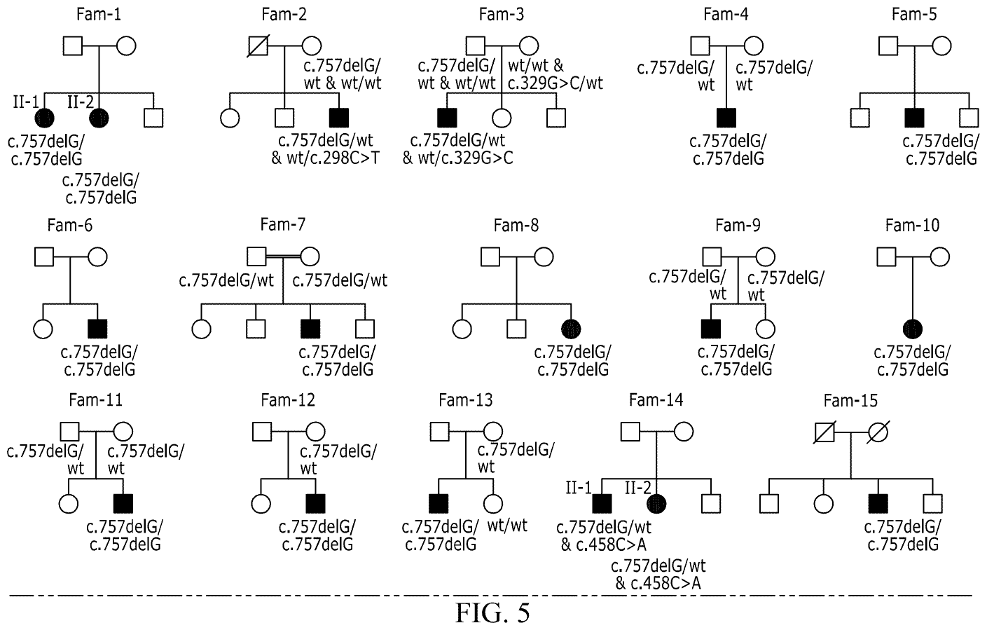

Inherited neuropathies, including Charcot-Marie-Tooth disease (CMT), represent an umbrella concept for clinically and genetically heterogeneous conditions affecting the peripheral nerves. CMT is classified depending on conduction velocity as demyelinating (CMT1) and axonal (CMT2) types. Distal hereditary motor neuropathy (dHMN) represents a form of CMT2 in which the burden of disease falls predominantly or exclusively on motor nerves (Rossor, Tomaselli, and Reilly 2016). As opposed to CMT1, for which over 90% of cases have mutations in known genes, only 20 to 30% of CMT2 and distal HMN patients receive a genetic diagnosis (Fridman et al. 2015). Since up to 70% of CMT2 and dHMN cases are sporadic, it becomes more challenging to identify candidate pathogenic genes from single case whole exome and genomic sequences; therefore, large collective datasets are necessary. Using the data aggregation of over 1,100 CMT whole exome sequencing (WES) and whole genome sequencing (WGS) available at the GENESIS analysis platform provided the largest collection of such high quality data available (Gonzalez et al. 2015). Genes with significant DNA variants present in multiple families were identified, as well as individual alleles overrepresented in CMT cases. When querying a subset of 598 undiagnosed CMT patients for recessive non-sense variants in genes shared by >3 families and with minor allele frequency in the gnomAD control database of <1%, 12 cases were identified from 11 unrelated families carrying a homozygous c.757delG; p.(Ala253GlnfsTer27) mutation in SORD. Four more cases from three unrelated families carried the heterozygous c.757delG; p.(Ala253GlnfsTer27) variant together with a second variant, c.298C>T; p.(Arg100Ter) in family 2 c.329G>C; p.(Arg110Pro) in family 3, and c.458C>A; p.(Ala153Asp) in II-1 and II-2 of family 14 (FIG. 1A-D and FIG. 5). All mutations represented loss-of-function (LOF) alleles except c.329G>C; p.(Arg110Pro). Interestingly, the Arg110Pro change is adjacent to the previously reported Tyr111Phe (corresponding to Tyr110Phe in rat), which was shown to abolish SORD enzymatic activity and destabilize the protein (Hellgren et al. 2007).

Interestingly, SORD has a non-functional highly homologous paralogue, the pseudogene SORD2P, which is thought to arise from the duplication of SORD within a 0.5 Mb region on chromosome 15 (Carr et al. 2016) (FIG. 1E). In order to specifically amplify SORD, but not SORD2P, in Sanger confirmation studies, primers were designed that took advantage of nucleotide sequence differences and distinct retrotransposon insertions in both genic regions (FIG. 5). Notably, the c.757delG; p.(Ala253GlnfsTer27) mutation in exon 7 of SORD is fixated in the pseudogene SORP2P in over 95% of control chromosomes, along with numerous additional exonic indel mutations, which prevent effective translation of SOPR2P (1000 Genomes Project Consortium et al. 2015; Lek et al. 2016). Because of the high similarity of the regions, a nested PCR approach was necessary to obtain specific amplification of exon 7 of SORD and distinguish it from the homologous region in SORD2P. The presence of the variants detected by WES was confirmed by Sanger sequencing in all cases and segregation data in immediate relative carriers was provided. (FIG. 1F and FIG. 5).

An independent set of 103 unresolved CMT2/dHMN cases WES at the (UCL Institute of Neurology in London (UK)) were screened. Nine cases from six unrelated families were identified carrying the homozygous c.757delG; p.(Ala253GlnfsTer27) mutation in SORD (8.7%). A third independent set of 297 recessive or sporadic CMT2/dHMN patients was screened by targeted Sanger sequencing of exon 7 of SORD, which was extended to the other coding exons if one c.757delG; p.(Ala253GlnfsTer27) was identified, and revealed 20 additional cases (7%) from 18 families with biallelic mutations in SORD: 16 cases with a homozygous c.757delG; p.(Ala253GlnfsTer27) mutation and four cases with c.757delG; p.(Ala253GlnfsTer27) in compound heterozygous state with a second likely pathogenic variant. The latter included c.964G>A; p.(Val322Ile) in family 29, a 275bp deletion c.316_425+165del in exon 4 in family 30, a de novo c.28C>T; p.(Leu10Phe) in family 32, and c.895C>T; p.(Arg299Ter) in family 33. All changes have a minor allele frequency (MAF) of <0.0001 in gnomAD (Lek et al. 2016). The residues affected by missense mutations are highly conserved across multiple species (FIG. 1D) with GERP scores greater than 3. Further, biallelic non-sense variants in SORD were absent from 4,598 index cases affected by distinct neurological disorders other than CMT present in the GENESIS database.

The allelic carrier frequency of the c.757delG; p.(Ala253GlnfsTer27) variant in the normal population is 0.003% based on an allelic count of 94 out of 30,872 in gnomAD genomes (Lek et al. 2016). Of note, the gnomAD exome set detected the c.757delG; p.(Ala253GlnfsTer27) change at a significantly lower rate at MAF=0.00008, due to failure to pass random forest filters. GENESIS uses the FreeBayes software for variant calling (Gonzalez et al. 2015), which may have resulted in an allele frequency closer to the gnomAD genome based call set (MAFGENESIS=0.002, 22 out of 9,196). Sanger sequencing of 600 healthy controls was performed, including 200 samples of European, 100 samples of Turkish and 200 samples of Middle Eastern origin, and identified three heterozygous, but no homozygous, c.757delG; p.(Ala253GlnfsTer27) alleles (MAF=0.0025). These calculations support an estimated prevalence of the homozygous c.757delG; p.(Ala253GlnfsTer27) allele alone of ˜1/100,000 individuals, making it the most common individual pathogenic allele in axonal neuropathies and one of the most common alleles for any Mendelian disease.

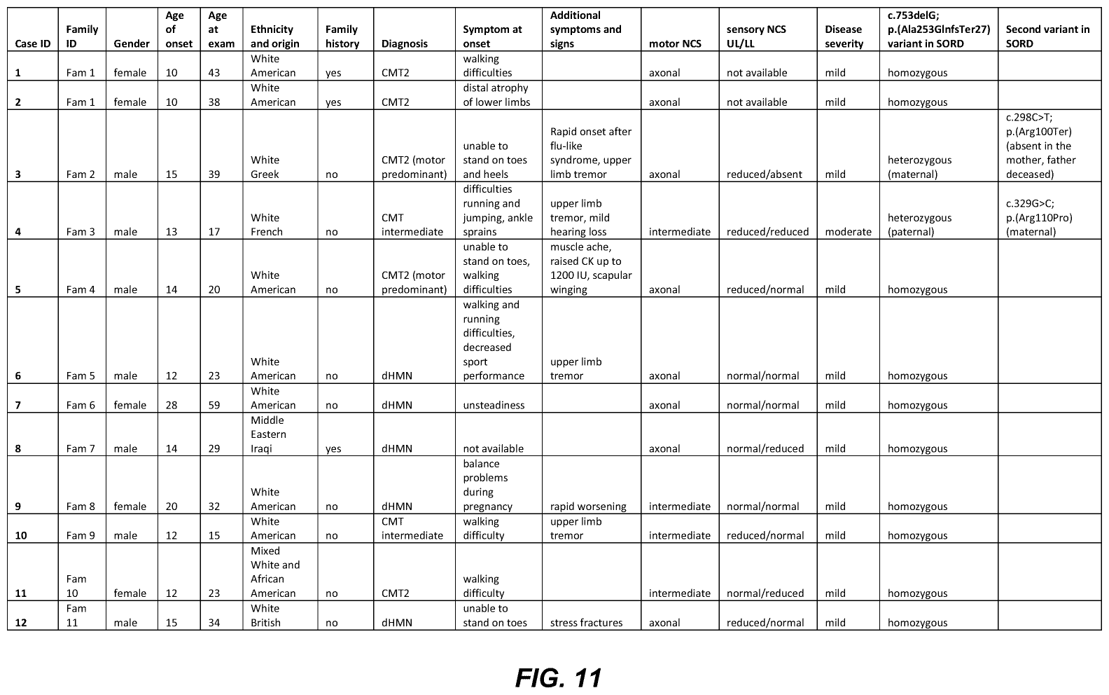

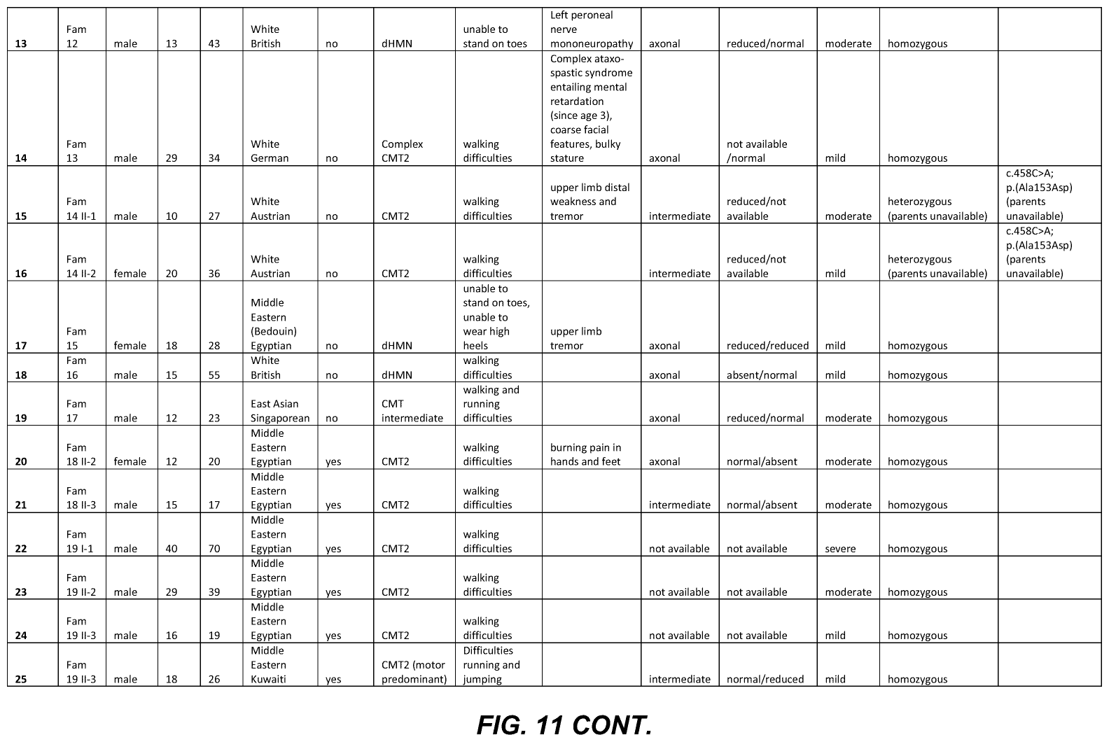

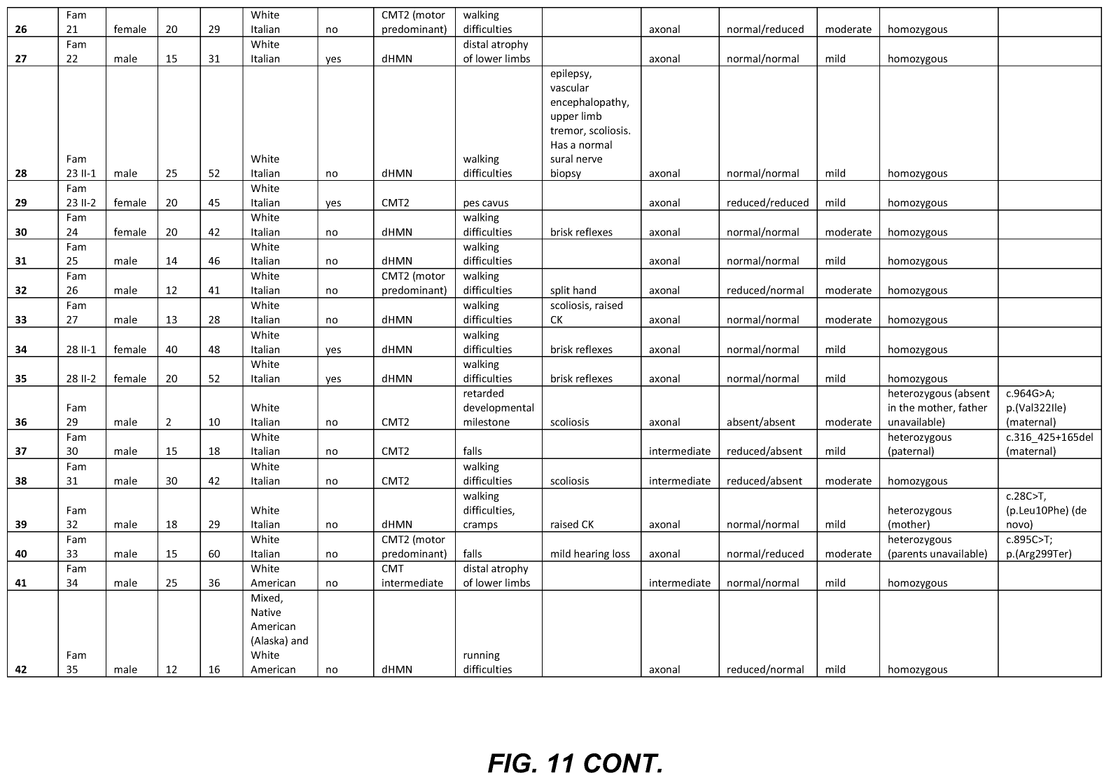

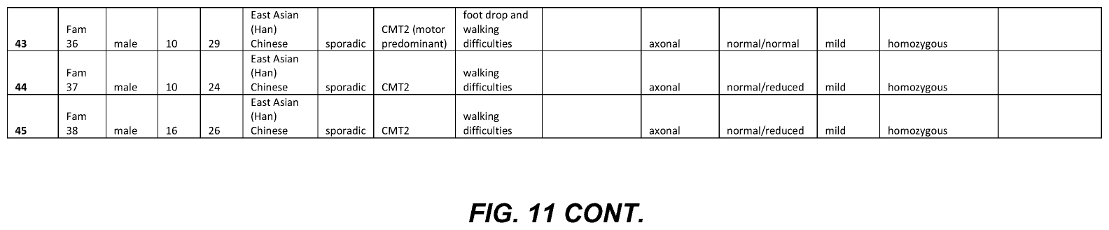

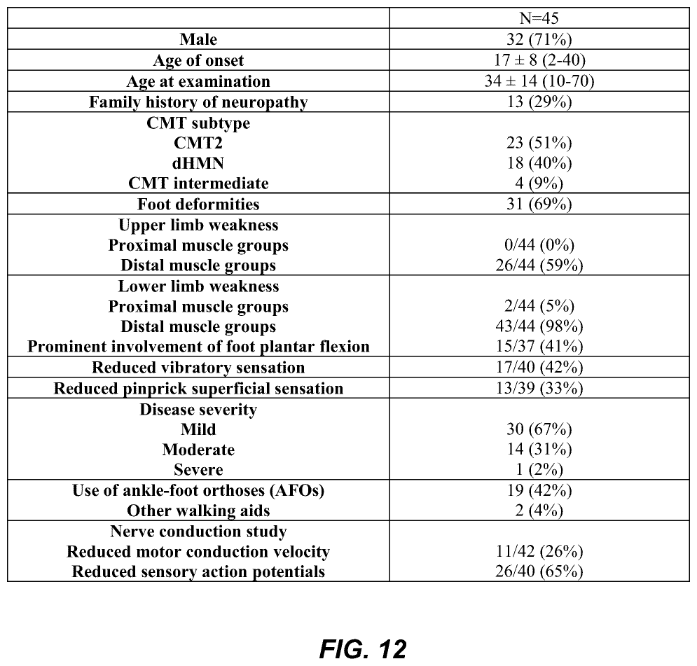

Overall 45 individuals affected by hereditary neuropathy from 38 unrelated families were identified in the present study to carry biallelic mutations in SORD (FIGS. 11 and 12). Of note, 71% of cases were sporadic with no evidence of family history or consanguinity. The formal clinical diagnosis was axonal CMT in 51% (n=16), distal HMN in 40% (n=18), and intermediate CMT in 9% (n=4) of cases The mean age of onset of the neuropathy was 17±8 years and walking difficulties was the most common complain at onset. Delayed motoric development milestones were uncommon, but two thirds of the patients reported foot deformities, indicating that the neuropathy probably started earlier in life. At first examination all individuals had limb weakness, but only half showed sensory impairment. Weakness was mild in distal upper limbs and ranged from mild to near complete paralysis in the distal lower limbs. Proximal muscles of the upper and lower limbs were typically unaffected. Seven patients had upper limb tremor, four had mild scoliosis and two had mild hearing loss. One case had a concurrent and likely unrelated syndromic disorder encompassing dysmorphic features, non-progressive mental retardation since the age of three years, and spastic ataxia with evidence of cerebellar atrophy at brain MRI. None of the patients had cataract nor involvement of other organs. According to the CMT neuropathy score, the neuropathy was mild in 67% (n=30), moderate in 31% (n=14) and severe in one case. 42% of patients (n=19) needed ankle-foot orthosis to sustain feet during walking, one patient required unilateral support and one patient was wheelchair dependent. Detailed nerve conduction studies were available in 42 patients and invariably showed a motor axonal neuropathy, with intermediate reduction of conduction velocities in 26% (n=11) and decreased sensory action potentials in 26% (n=65).

### Example 2: Assessing SORD Protein Expression in Human Fibroblasts and SORD Levels in Blood

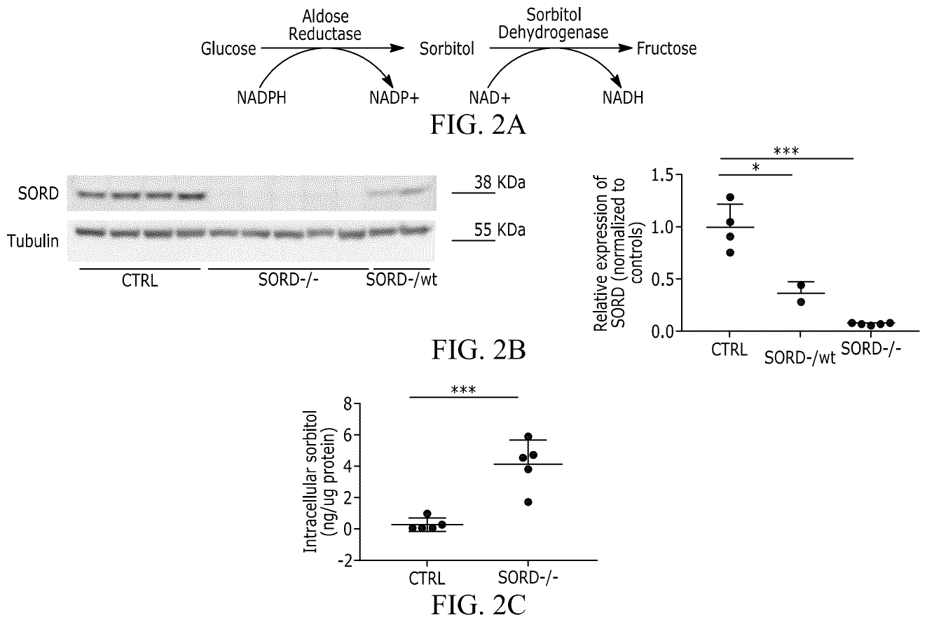

Sorbitol dehydrogenase is a homotetrameric enzyme of 38-kDa subunits, which is widely distributed in mammalian tissues (Johansson et al. 2001; Hellgren et al. 2007; Lindstad, Teigen, and Skjeldal 2013). It represents the second enzyme of the two-step polyol pathway, in which glucose is converted to sorbitol, a relatively non-metabolizable sugar, by the enzyme aldose reductase (AR). Sorbitol is then oxidized to fructose by SORD (FIG. 2A). To gather further insights into the functional consequences of recessive mutations in SORD, next SORD expression was assessed in fibroblasts from five unrelated affected individuals with homozygous c.757delG; p.(Ala253GlnfsTer27) (n=4) or c.757delG; p.(Ala253GlnfsTer27) & c.895C>T; p.(Arg299Ter) (n=1) variants as well as two unaffected carriers of c.757delG; p.(Ala253GlnfsTer27) in heterozygous state. SORD protein was absent in all patients and the wild-type levels were reduced in unaffected carriers compared to controls (FIG. 2B). Accordingly, intracellular sorbitol concentrations were over 10 times higher in patients' fibroblasts compared to controls, in keeping with a loss of SORD enzymatic activity (FIG. 2C). Fasting sorbitol levels in serum from ten patients carrying the homozygous p.Ala253GlnfsTer27 mutation and ten unrelated controls and found it was over 100 times higher (14.82±0.780 vs 0.046±0.004 mg/L, p<0.0001) was determined, confirming the lack of SORD enzymatic activity in patients (FIG. 13). This study also demonstrates that sorbitol is a useful marker for detecting or characterizing inherited neuropathy associated with SORD mutation in a mammalian subject.

### Example 3: Investigating the SORD Mutation in Models of SORD Deficiency

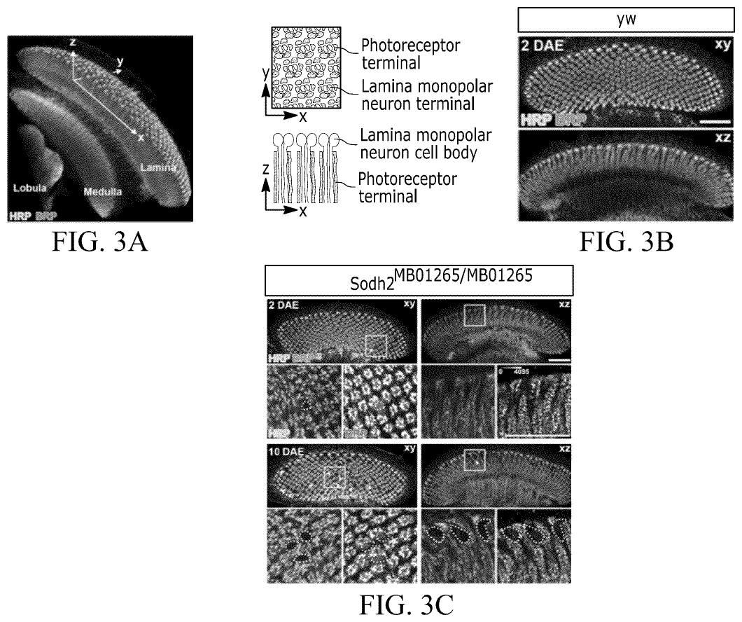

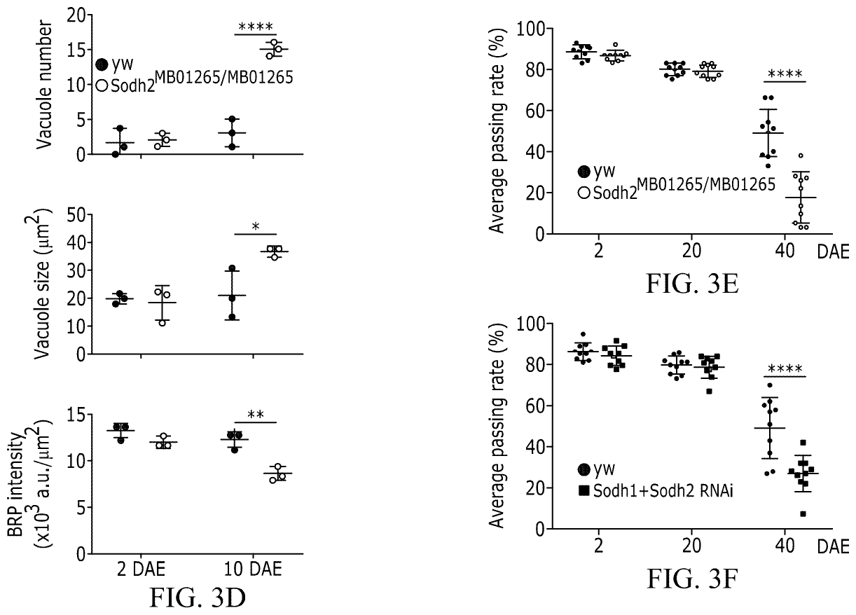

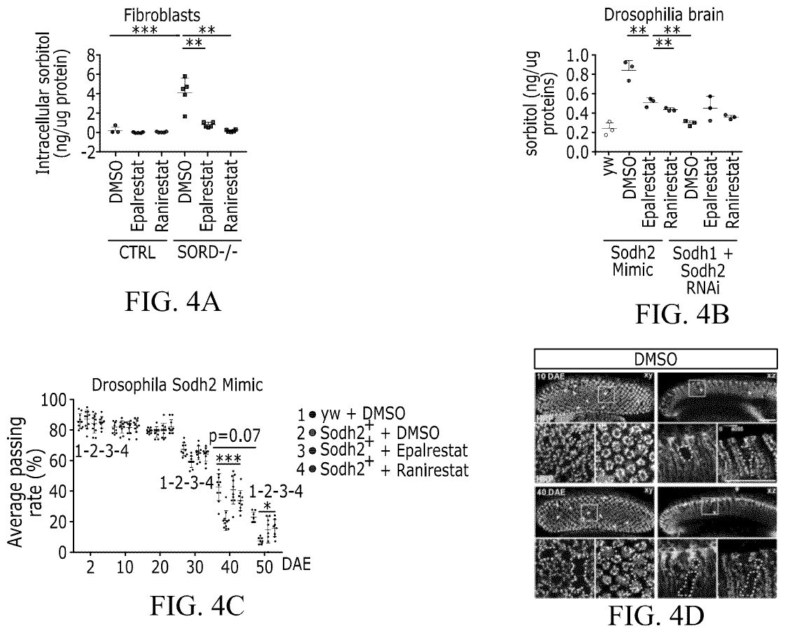

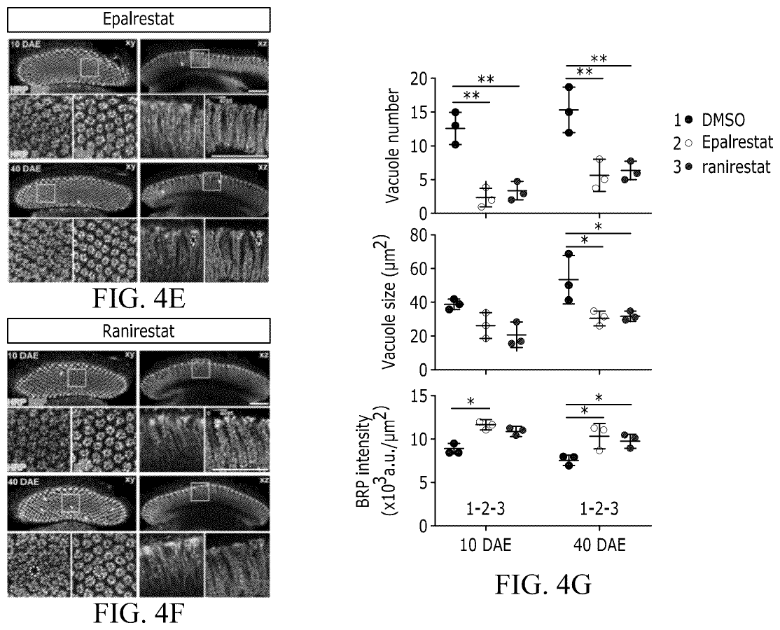

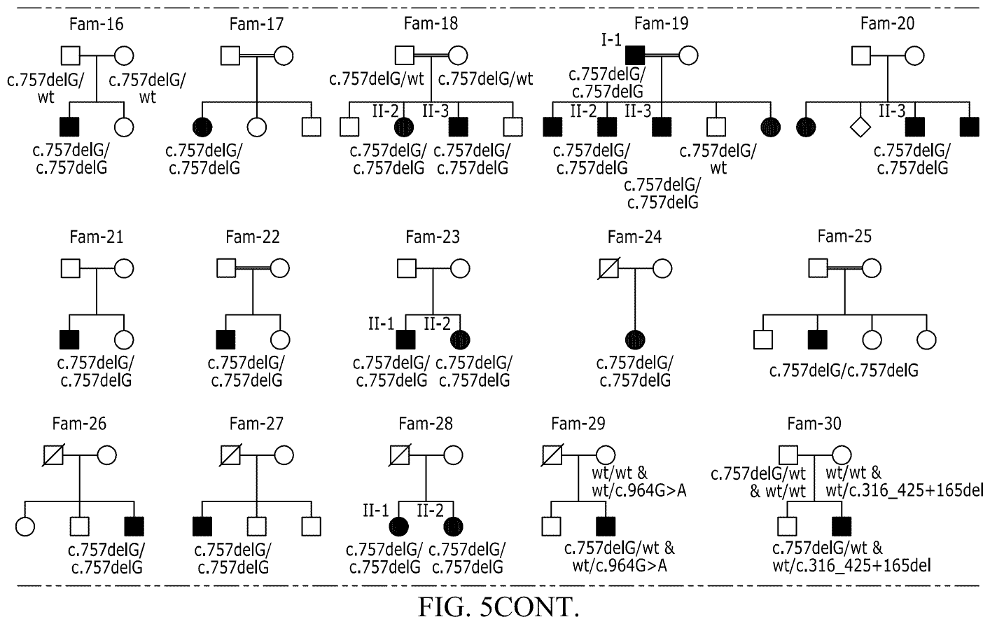

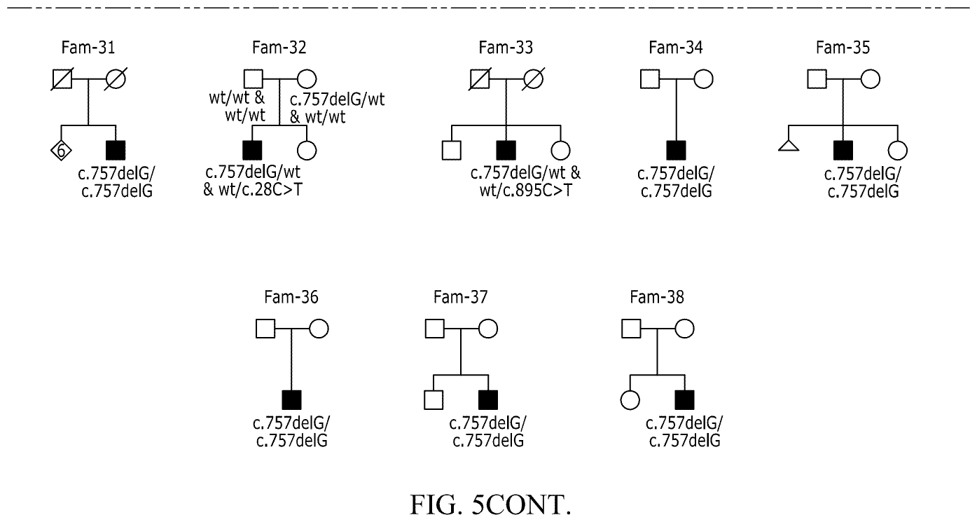

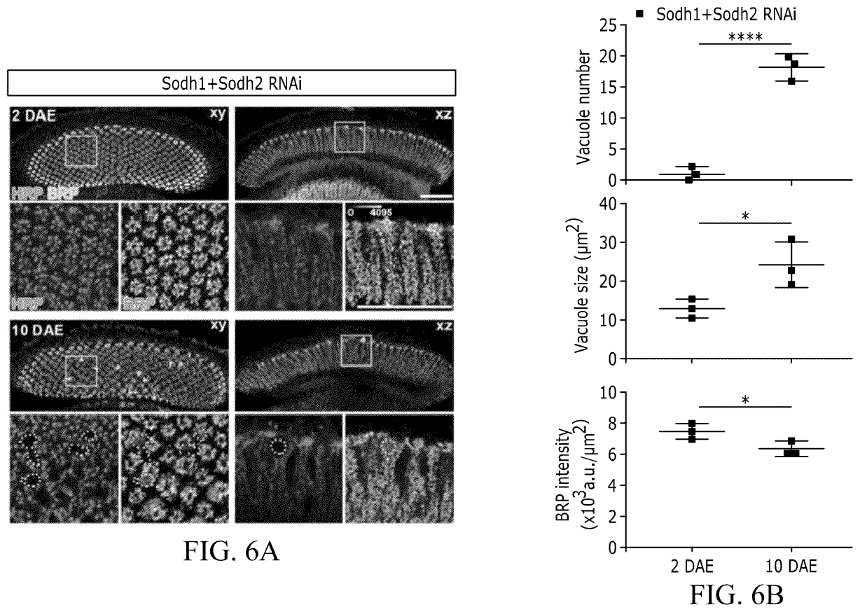

To further explore the pathophysiology of SORD mutation in vivo, Drosophila melanogaster models of SORD deficiency were established. Drosophila has two functional SORD genes (Sodh1 and Sodh2) that share 90% residue identity (Luque et al. 1998). SORD is conserved across distant phyla and Drosophila Sodh1 (NP_001287203.1) and Sodh2 (NCBI Reference Sequence: NP_524311.1) encoded proteins share 75% and 73% identity with human SORD protein (NCBI Reference Sequence: NP_003095.2 (SEQ ID NO: 46)), respectively. A mutant allele of Sodh2 was obtained where the gene is disrupted by a transposon Minos mediated integration cassette (MiMIC) insertion (Sodh2^{MB01265}) (Bellen et al. 2011). Homozygous Sodh2 (Sodh2^{MB01265/MB01265}) mutants are viable with normal life span. To characterize neurodegenerative phenotypes, the Drosophila visual system was used to take advantage of the highly organized parallel axons of the compound eye that allow in vivo detection of subtle neuronal and synaptic pathological changes (Bausenwein, Dittrich, and Fischbach 1992). Axons of the outer photoreceptor axons traverse the lamina cortex and make synaptic connections with lamina monopolar neurons in the lamina layer (FIG. 3A). In the control flies (yw) at 2 days after eclosion (DAE), the organized lamina cartridges of photoreceptor synapses can be visualized in the xy- and xz-planes, respectively (FIG. 3B). A loss of photoreceptor terminals in the lamina layer of Sodh2^{MB01265/MB01265} mutants was observed at 2 days after eclosion (DAE) (FIG. 3C). The phenotype became progressively severe at 10 DAE, with vacuoles being more numerous and larger in size distributed across the synaptic lamina layer (FIG. 3C, D). These vacuoles exhibited a loss of neuronal membrane (marked by HRP labelling), as well as a reduced Bruchpilot (BRP, a synaptic active zone cytomatrix protein) labelling, indicating synaptic degeneration (FIG. 3C, D). To validate the findings described herein, a second SORD) model was generated by specific knockdown of both Sodh1 and Sodh2 expression in neurons using a pan-neuronal driver elav^{C155}. Loss of both Sodh1 and Sodh2 resulted in age-dependent synapse degeneration, similar to that of homozygous Sodh2 (Sodh2^{MB01265/MB01265}) (FIG. 6). The behavioral phenotypes of SORD deficiency the Sodh2^{MB01265/MB01265} homozygous flies with a systemic loss of function in Sodh2 were characterized and although these flies exhibited a normal life-span, their locomotor activity was significantly compromised at a late-stage (40 DAE) (FIG. 3E, F). This indicated a progressive, age-dependent neuromuscular dysfunction reminiscent of hereditary neuropathies. Moreover, the sorbitol levels were measured in fly heads at 10 DAE and observed a significant increase in the Sodh2^{MB01265/MB01265} model (FIG. 4B), consistent with the observation in patient fibroblasts. Taken together, Drosophila models of SORD deficiency were successfully established that recapitulate typical pathological phenotypes in human patients, including (1) a normal lifespan, (2) progressive and age-dependent synaptic degeneration and locomotor deficiency, and (3) increased sorbitol levels.

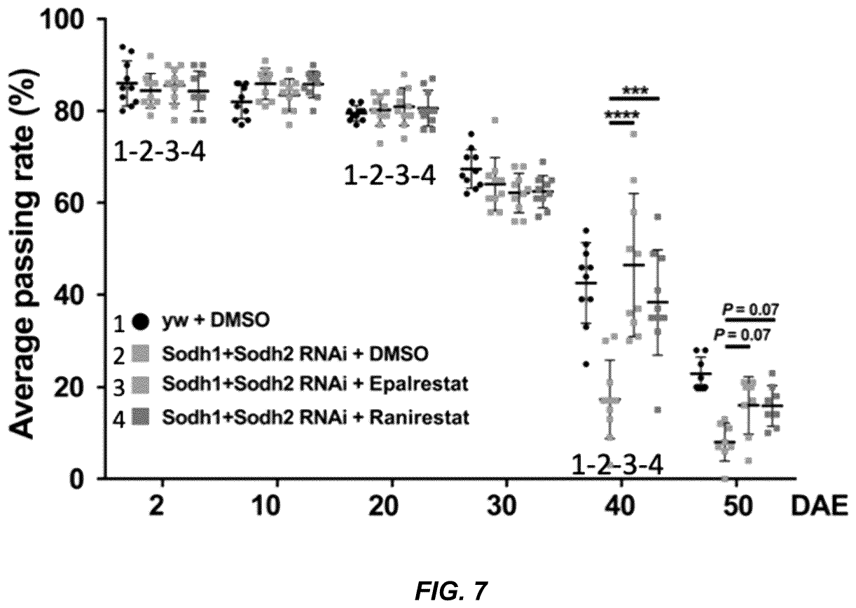

After establishing loss-of-function as the mechanism of action and a known enzymatic pathway, treatment options for SORD associated hereditary neuropathy were investigated. It had previously been shown that the pharmacological inhibition of aldose reductase, the enzyme upstream of SORD, represents a successful strategy to reduce toxic sorbitol accumulation in cellular and animal model of diabetes (Kikkawa et al. 1983; Matsumoto et al. 2008; Ramirez and Borja 2008; Hao et al. 2015; Grewal et al. 2016) and, arguably, also humans (Chalk, Benstead, and Moore 2007; Polydefkis et al. 2015; Sekiguchi et al. 2019). The effect of two commercially available aldose reductase inhibitors (ARI), Epalrestat and Ranirestat, were tested on intracellular sorbitol accumulation in patient fibroblasts lacking functional SORD. Patient and control fibroblasts were grown for 72 hrs in the presence or absence of Epalrestat (100 μM) or Ranirestat (10 μM) and intracellular sorbitol levels were measured thereafter. Both ARI, Epalrestat and Ranirestat, achieved a significant reduction of sorbitol to a level comparable to controls (FIG. 4A). Further, Drosophila models of SORD were fed with Epalrestat and Ranirestat starting at 2 DAE. A significant reduction of sorbitol level was observed in the Sodh2^{MB01265/MB0126} fly heads at 10 DAE (FIG. 4B). Importantly, the locomotor activities of Sodh2^{MB01265/MB01265} flies and flies with neuronal specific knockdown of both Sodh1 and Sodh2 were rescued to the levels of yw control flies (FIG. 4C, FIG. 7). Furthermore, Epalrestat or Ranirestat feeding restored the age-dependent synaptic defects in Sodh2^{MB01265/MB01265} mutant flies. In DMSO vehicle treated flies, the loss of synaptic termini was highly prominent in the advanced age of 40 DAE where the expansion of neighboring vacuoles resulted in fused, much larger vacuoles encompassing multiple synaptic cartridges (FIG. 4D). Remarkably, epalrestat/ranirestat feeding reduced the number of vacuoles and restored the localization of synaptic cytomatrix protein BRP at both 10 and 40 DAE (FIG. 4E-G).

In summary, SORD represents a novel recessive gene causing axonal/intermediate, motor predominant CMT. Genetic data from the cohort as well as from control databases suggest that the predominant pathogenic variant in SORD, c.757delG; p.(Ala253GlnfsTer27), with a carrier frequency in of ˜3/1,000 individuals in the population, may represent one of the most common specific alleles causing a recessive Mendelian disease. Indeed, with a frequency in undiagnosed CMT2 and dHMN cases of up to ˜10%, it will likely account for a significant portion of the diagnostic gap in inherited axonal neuropathies. It is intriguing that, despite their frequency, mutations in SORD) were not identified as a cause of CMT by previous studies. The presence of the human SORD2P gene duplication may have hampered the detection of variants in the functional SORD, since available annotation programs are highly dependent on the unique mapping of 150-300 bp long reads generated by current next-generation sequencing technologies. Other known pathogenic variants have previously been shown to be concealed by the presence of pseudogenes (De Vos et al. 2004). The pathogenicity of SORD) mutations is further supported by in vitro data in patient-derived fibroblasts, which showed absent SORD protein and intracellular sorbitol accumulation. Two in vivo Drosophila models recapitulated the human phenotype with progressive synaptic degeneration and motor impairment, SORD deficiency, and in increased sorbitol levels.

The studies described herein demonstrate that enzymatic loss-of-function and subsequent sorbitol accumulation is a mechanism of action for SORD associated CMT. Previous studies in cellular and animal models of diabetes have shown that an increased polyol influx with intracellular sorbitol accumulation is paralleled by an increase in cellular osmolarity, oxidative stress and decreased NADPH levels, which can all have a detrimental effects on peripheral nerves (Schmidt et al. 2001; Obrosova 2005; Sango et al. 2006). However, previous studies on adult C57BL/LiA mice expressing reduced level of SORD protein due to an intronic splicing mutation did not identify overt neurological defects (Holmes, Duley, and Hilgers 1982; Lee, Chung, and Chung 1995; Ng et al. 1998). Based on patient clinical data and the late-onset phenotype in flies, it will be important to extend the observation to aging C57BL/LiA mice or create complete knock-out SORD mouse or rat models. The study further unravels a central role of the polyol pathway in peripheral nerve metabolism and survival in normoglycemic conditions. Although the mechanism by which intracellular sorbitol accumulation can lead to selective degeneration of peripheral nerves is yet unknown, the observation of increased sorbitol levels in patient derived cells in this study has promising implications, both as a biomarker of the disease and as a target of future therapeutic interventions, including methods for substrate reduction, gene replacement or correction, and SORD enzyme substitution. Accordingly, disclosed herein are preclinical studies demonstrating the beneficial effects of substrate reduction via ARI application in human derived cells and Drosophila models. Epalrestat is currently marketed in few countries for the treatment of diabetic complications (Grewal et al. 2016) while Ranirestat has been advanced into late stages of clinical trials (Polydefkis et al. 2015; Sekiguchi et al. 2019).

### REFERENCES

Auton, et al. 1000 Genomes Project Consortium, 2015. “A Global Reference for Human Genetic Variation.” Nature 526 (7571): 68-74.
Bausenwein, et al. 1992. “The Optic Lobe of Drosophila Melanogaster. II. Sorting of Retinotopic Pathways in the Medulla.” Cell and Tissue Research 267 (1): 17-28.
Bellen, et al. 2011. “The Drosophila Gene Disruption Project: Progress Using Transposons with Distinctive Site Specificities.” Genetics 188 (3): 731-43.
Brazill, et al. 2018. “Quantitative Cell Biology of Neurodegeneration in Drosophila Through Unbiased Analysis of Fluorescently Tagged Proteins Using ImageJ.” Journal of Visualized Experiments: JoVE, no. 138 (03).
Callaghan, et al. 2012. “Diabetic Neuropathy: Clinical Manifestations and Current Treatments.” The Lancet. Neurology 11 (6): 521-34.
Carr, et al. 2016. “A Study of the Neuropathy Associated with Transthyretin Amyloidosis (ATTR) in the UK.” Journal of Neurology, Neurosurgery, and Psychiatry 87 (6): 620-27.
Chalk, et al. 2007. “Aldose Reductase Inhibitors for the Treatment of Diabetic Polyneuropathy.” The Cochrane Database of Systematic Reviews, no. 4 (October): CD004572.
De Vos, et al. 2004. “Novel PMS2 Pseudogenes Can Conceal Recessive Mutations Causing a Distinctive Childhood Cancer Syndrome.” American Journal of Human Genetics 74 (5): 954-64.
Dyck, et al. 1993. “The Prevalence by Staged Severity of Various Types of Diabetic Neuropathy, Retinopathy, and Nephropathy in a Population-Based Cohort: The Rochester Diabetic Neuropathy Study.” Neurology 43 (4): 817-24.
Fridman, et al. 2015. “CMT Subtypes and Disease Burden in Patients Enrolled in the Inherited Neuropathies Consortium Natural History Study: A Cross-Sectional Analysis.” Journal of Neurology, Neurosurgery, and Psychiatry 86 (8): 873-78.
Gonzalez, et al. 2015. “Innovative Genomic Collaboration Using the GENESIS (GEM.App) Platform.” Human Mutation 36 (10): 950-56.
Grewal, et al. 2016. “Updates on Aldose Reductase Inhibitors for Management of Diabetic Complications and Non-Diabetic Diseases.” Mini Reviews in Medicinal Chemistry 16 (2): 120-62.
Hao, et al. 2015. “Hyperglycemia Promotes Schwann Cell De-Differentiation and De-Myelination via Sorbitol Accumulation and Igf1 Protein Down-Regulation.” The Journal of Biological Chemistry 290 (28): 17106-15.
Hellgren, et al. 2007. “A Hydrogen-Bonding Network in Mammalian Sorbitol Dehydrogenase Stabilizes the Tetrameric State and Is Essential for the Catalytic Power.” Cellular and Molecular Life Sciences: CMLS 64 (23): 3129-38.
Holmes, et al. 1982. “Sorbitol Dehydrogenase Genetics in the Mouse: A ‘null’ Mutant in a ‘European’ C57BL Strain.” Animal Blood Groups and Biochemical Genetics 13 (4): 263-72.
Johansson, et al. 2001. “Crystal Structure of Sorbitol Dehydrogenase.” Chemico-Biological Interactions 130-132 (1-3): 351-58.
Kikkawa, et al. 1983. “Effect of a New Aldose Reductase Inhibitor, (E)-3-Carboxymethyl-5-[(2E)-Methyl-3-Phenylpropenylidene]Rhodanine (ONO-2235) on Peripheral Nerve Disorders in Streptozotocin-Diabetic Rats.” Diabetologia 24 (4): 290-92.
Lee, et al. 1995. “Demonstration That Polyol Accumulation Is Responsible for Diabetic Cataract by the Use of Transgenic Mice Expressing the Aldose Reductase Gene in the Lens.” Proceedings of the National Academy of Sciences of the United States of America 92 (7): 2780-84.
Lek, et al. 2016. “Analysis of Protein-Coding Genetic Variation in 60,706 Humans.” Nature 536 (7616): 285-91.
Lindstad, et al. 2013. “Inhibition of Sorbitol Dehydrogenase by Nucleosides and Nucleotides.” Biochemical and Biophysical Research Communications 435 (2): 202-8.
Luque, et al. 1998. “Sorbitol Dehydrogenase of Drosophila. Gene, Protein, and Expression Data Show a Two-Gene System.” The Journal of Biological Chemistry 273 (51): 34293-301.
Matsumoto, et al. 2008. “Long-Term Treatment with Ranirestat (AS-3201), a Potent Aldose Reductase Inhibitor, Suppresses Diabetic Neuropathy and Cataract Formation in Rats.” Journal of Pharmacological Sciences 107 (3): 340-48.
Ng, et al. 1998. “Effects of Sorbitol Dehydrogenase Deficiency on Nerve Conduction in Experimental Diabetic Mice.” Diabetes 47 (6): 961-66.
Obrosova I. 2005. “Increased Sorbitol Pathway Activity Generates Oxidative Stress in Tissue Sites for Diabetic Complications.” Antioxidants & Redox Signaling 7 (11-12): 1543-52.
Polydefkis, et al. 2015. “Safety and Efficacy of Ranirestat in Patients with Mild-to-Moderate Diabetic Sensorimotor Polyneuropathy.” Journal of the Peripheral Nervous System: JPNS 20 (4): 363-71.
Ramirez, et al. 2008. “Epalrestat: An Aldose Reductase Inhibitor for the Treatment of Diabetic Neuropathy.” Pharmacotherapy 28 (5): 646-55.
Rossor, et al. 2016. “Recent Advances in the Genetic Neuropathies.” Current Opinion in Neurology 29 (5): 537-48.
Sango, et al. 2006. “High Glucose-Induced Activation of the Polyol Pathway and Changes of Gene Expression Profiles in Immortalized Adult Mouse Schwann Cells IMS32.” Journal of Neurochemistry 98 (2): 446-58.
Schmidt, et al. 2001. “Inhibition of Sorbitol Dehydrogenase Exacerbates Autonomic Neuropathy in Rats with Streptozotocin-Induced Diabetes.” Journal of Neuropathology and Experimental Neurology 60 (12): 1153-69.
Sekiguchi, et al. 2019. “Aldose Reductase Inhibitor Ranirestat Significantly Improves Nerve Conduction Velocity in Diabetic Polyneuropathy: A Randomized Double-Blind Placebo-Controlled Study in Japan.” Journal of Diabetes Investigation 10 (2): 466-74.

What is claimed:

1. A method of treating inherited neuropathy in a mammalian subject, the method comprising:
(a) detecting the presence of a mutation in the sorbitol dehydrogenase (SORD) gene in a sample from the subject; and
(b) administering to the subject a polynucleotide that encodes a SORD peptide, an aldose reductase antisense oligonucleotide, an agent that blocks expression of a mutant SORD gene, an agent that corrects the mutation in SORD gene, or a combination of any of the foregoing.

2. The method of claim 1, wherein the method comprises administering a polynucleotide encoding the SORD peptide.

3. The method of claim 1, wherein the method comprises administering an agent that corrects the mutation in the SORD gene, wherein the agent is a CRISPR Cas9 protein and one or more guide RNA molecules.

4. A method of treating inherited neuropathy in a mammalian subject, the method comprising:
(a) detecting the presence of a mutation in the sorbitol dehydrogenase (SORD) gene in a sample from the subject; and
(b) administering to the subject Alrestatin, Epalrestat, Diepalrestat, Fidarestat, Imirestat, Lidorestat, Minalrestat, Ponalrestat, Ranirestat, Salfredin B₁₁, Sorbinil, Tolrestat, Zenarestat, or Zopolrestat.

5. A method of treating inherited neuropathy in a mammalian subject, the method comprising:
(a) detecting the presence of a mutation in the sorbitol dehydrogenase (SORD) gene in a sample from the subject; and
(b) administering to the subject a SORD peptide.

6. The method of any one of claims 1-5, wherein the mutation in the SORD gene is c.753delG; p.Ala253GlnfsTer27, c.329G>C; p.Arg110Pro, c.298C>T; p.Arg100Ter, or c.458C>A; p.Ala153 Asp.

7. The method of any one of claims 1-5, wherein the mutation in the SORD gene is c.757delG; p.Ala253GlnfsTer27, c.28C>T; p.Leu10Phe, c.316_425+165del; p.Cys106Ter, c.295C>T; p.Arg299Ter, c.964G>A; p. Val322Ile, or a deletion of individual or multiple coding exons or the entire SORD gene.

8. The method of any one of claims 1-7, further comprising measuring sorbitol in a sample from the subject.

9. A method of characterizing a neuropathy in a mammalian subject, the method comprising measuring the level of sorbitol in a subject suffering from a neuropathy, wherein a sorbitol level of greater than about 10 g/L indicates that the neuropathy is associated with a mutation in the sorbitol dehydrogenase (SORD) gene.

10. A method of evaluating the efficacy of a treatment for an inherited neuropathy in a subject, the method comprising
administering to the subject an agent selected from the group consisting of an aldose reductase inhibitor, an aldose reductase antisense oligonucleotide, a polynucleotide that encodes a SORD peptide, a SORD peptide, an agent that blocks expression of a mutant SORD gene, and an agent that corrects the mutation in SORD gene, or a combination of any of the foregoing; and
measuring the level of sorbitol in a subject.
